(19) United States
(12) Patent Application Publication
(10) Pub. No.: US 2024/0293232 A1
(43) Pub. Date: Sep. 5, 2024

(54) INSTRUMENTS AND METHODS FOR POSTERIOR SURGICAL APPROACH FOR SACROILIAC JOINT

(71) Applicant: Omnia Medical, LLC, Morgantown, WV (US)

(72) Inventors: Troy Schifano, Morgantown, WV (US); Steve Anderson, Folsom, CA (US); Abigail Lorraine Mann, Morgantown, WV (US); Farhan Danial Bin Zahidi, Morgantown, WV (US)

(73) Assignee: Omnia Medical, LLC, Morgantown, WV (US)

(21) Appl. No.: 18/433,030

(22) Filed: Feb. 5, 2024

Related U.S. Application Data
(63) Continuation-in-part of application No. 16/816,258, filed on Mar. 12, 2020.
Application No. 16/816,258 is a continuation of application No. 16/810,823, filed on Mar. 5, 2020, now abandoned.
(60) Provisional application No. 62/813,773, filed on Mar. 5, 2019.
(60) Provisional application No. 63/443,052, filed on Feb. 3, 2023.
(60) Provisional application No. 63/452,841, filed on Mar. 17, 2023.
(60) Provisional application No. 63/621,260, filed on Jan. 16, 2024.

(51) Int. Cl. A61F 2/30; A61B 17/16; A61B 17/86; A61B 17/88

(52) U.S. Cl. CPC A61F 2/30988; A61B 17/1615; A61B 17/8695; A61B 17/8875; A61F 2002/30289; A61F 2002/30433; A61F 2002/30622; A61F 2002/30841; A61F 2002/3093; A61F 2002/30995

(57) ABSTRACT

A hybrid washer tower is provided that includes a columnar outer wall extending from a distal end to a proximal end. The columnar outer wall defines a hollow barrel that extends from the distal end to the proximal end so that the hollow barrel being at least partially enclosed by the outer wall. The hybrid washer tower also includes at least one lateral tang positioned on and projecting from a terminal portion of the distal end of the outer wall to engage with surrounding bone or tissue. Furthermore, the hybrid washer tower includes an interlock channel defined within the outer wall with an open section positioned at a terminal portion of the proximal end of the outer wall to accept a connection pin positioned on a surgical instrument and direct the movement of the surgical instrument as it progresses through the hollow barrel.

### CROSS-REFERENCE TO RELATED APPLICATION

This application is a continuation-in-part of and claims priority to U.S. application Ser. No. 16/816,258, filed Mar. 12, 2020, pending, which is a continuation of U.S. application Ser. No. 16/810,823, filed Mar. 5, 2020, abandoned, which is a non-provisional of U.S. Provisional Application No. 62/813,773, filed Mar. 5, 2019. This application is a continuation-in-part of and claims priority to U.S. application Ser. No. 17/864,367, filed Jul. 13, 2022, pending, which is a continuation-in-part of U.S. application Ser. No. 17/745,896, filed May 17, 2022, pending, which is a continuation of U.S. application Ser. No. 16/689,073, filed Nov. 19, 2019, abandoned, which is a continuation of U.S. application Ser. No. 14/668,976, filed Mar. 25, 2015 (issued May 4, 2021 as U.S. Pat. No. 10,993,757), which is a continuation-in-part of U.S. application Ser. No. 13/842,227, filed Mar. 15, 2013 (issued Sep. 1, 2015 as U.S. Pat. No. 9,119,732). This application is a continuation-in-part and claims priority to U.S. application Ser. No. 29/826,471, filed Feb. 12, 2022, pending, which is a continuation of U.S. application Ser. No. 29/718,879, filed Dec. 30, 2019, pending, which is a continuation-in-part of U.S. application Ser. No. 14/668,976, filed Mar. 25, 2015 (issued May 4, 2021 as U.S. Pat. No. 10,993,757), which is a continuation-in-part of U.S. application Ser. No. 13/842,227, filed Mar. 15, 2013 (issued Sep. 1, 2015 as U.S. Pat. No. 9,119,732). This application is also a non-provisional of U.S. Provisional Application No. 63/443,052, filed Feb. 3, 2023, U.S. Provisional Application No. 63/452,841, filed Mar. 17, 2023, and U.S. Provisional Application No. 63/621,260, filed Jan. 16, 2024, the entirety of which are incorporated herein.

### TECHNICAL FIELD

The present disclosure relates generally to medical devices and medical methods. More particularly, the present disclosure relates to musculoskeletal surgical methods and associated surgical tools for treatment of the sacroiliac joint.

### BACKGROUND

Lower back pain is a common ailment among the population and results in both pain and suffering as well as loss of work time. Thus, approaches for the treatment of back pain can both relieve suffering as well as reduce employee down time. Therefore, effective treatments for lower back pain have both economic benefits as well as the benefit of alleviating considerable suffering.

The sacroiliac joint is located in the lower back at the juncture of the ilium, the upper bone of the pelvis, and the sacrum at the base of the spine. While the sacroiliac joint has a limited range of motion, dysfunction of the joint has been identified. The joint is supported by a range of ligaments including, for example, the sacroiliac ligament at the base of the joint and the anterior sacroiliac ligament at the top of the joint.

High energy pelvic ring injuries that involve disruption of the SI joint and/or displaced fractures of the sacrum present unique challenges to the orthopedic traumatologist. Some sacral fractures require solid posterior stabilization, which may be difficult to achieve with typical treatment methods. Furthermore, vertically unstable sacral fractures/SI joint disruptions have a relatively high incidence of neurovascular injury and may require unique stabilization. Typically, a spinal surgeon will be involved to perform lumbo-pelvic stabilization of these injuries to provide vertical stability of the injury. However, there may be significant soft tissue trauma associated with these injuries, making extensive surgical approaches of elevated risk in terms of infection and wound complications.

Immobilization of the SI joint can result in significant relief of lower backpain. Current techniques and instrumentation systems may require extensive surgical exposure and dissection. Moreover, such instrumentation systems are typically designed for other applications, and not to connect and stabilize the lumbar spine and pelvis. As a result, this can make the surgical times longer and more frustrating for surgeons and surgical staff. For example, traditional posterior iliac screws are often prominent because the posterior iliac crest is relatively subcutaneous. Yet, this sometimes makes hardware painful for the patient and at risk for pressure soreness following surgery.

Furthermore, the current techniques and instruments do not allow for a secure and consistent fusion construct. They may provide one or the other many times, but not both issues. This may lead to further SI joint instability and a failed surgery.

It is therefore desirable to provide new surgical methods and tools for treating damaged sacroiliac joints that securely and consistently fuse the joint.

### SUMMARY

The present disclosure includes improved methods and devices for the immobilization or fusion of the Sacroiliac joint and apparatuses for facilitating the procedure. Immobilization may refer to mechanical holding and/or surgical fusion.

In one embodiment, the instruments of the present disclosure include a break-away washer for the posterior surgical stabilization of a sacroiliac joint. The washer includes a washer ring positioned at a distal end of the washer, and the washer ring further includes a first surface and a second surface configured to couple with the body of a fusion implant. The break-away washer also includes a lateral tang extending distally from the first surface to engage an area surrounding an implant site and stabilize the washer. Furthermore, the break-away washer includes at least one guidance tab affixed to the second surface of the washer ring that extends proximally to define a semi-enclosed, columnar structure and at least one corresponding break-away connection point extending proximally from the second surface that couples the guidance tab to the washer ring. Each of the break-away connection points can be separated from the washer ring following implantation, enabling removal of each guidance tab while leaving the washer ring positioned at the implant site.

In another embodiment, the present disclosure provides for a method for the posterior surgical stabilization of a sacroiliac joint using a break-away washer. The method includes a first step of creating an incision proximal to a patient's sacroiliac joint to allow access to a posterior region of the sacroiliac joint. The method includes a second step of locating an implant site and introducing a guide pin to mark the implant site. The method includes a third step of fitting a dilator over the guide pin and dilating the incision. The method includes a fourth step of threading a cannula over the dilator and progressing a distal end of the cannula along the length of the dilator into the dilated incision. The method includes a fifth step of removing the dilator and the guide pin through a channel of the cannula, leaving the hybrid washer tower positioned within the dilated incision to enable the passage of one or more surgical instruments and one or more implants through the channel to the implant site. The method includes a sixth step of inserting a drill through the channel until one or more blades of the drill are positioned proximate to the implant site, rotating the blades to create a void in the patient's sacroiliac joint, and then removing the drill through the channel. The method includes a seventh step of coupling a washer driver to a proximal end of a washer, the washer includes a washer ring positioned at a distal end of the washer, one or more guidance tabs, and one or more corresponding break-away connection points that affix the guidance tabs to the washer ring. The method includes an eighth step of inserting the distal end of the washer into the channel of the cannula and applying a driving force to a flat end of the washer driver to drive the distal end of the washer into the incision. The method includes a ninth step of coupling the implant to a distal end of an inserter, inserting the distal end of the inserter into the channel, and progressing the inserter through the channel until the implant extends through the washer ring such that a fixation element of the implant is positioned proximate to the void. The method includes a tenth step of rotating the inserter to cause the fixation element to affix the patient's sacroiliac joint within the void. The method includes an eleventh step of decoupling the implant from the inserter and removing the inserter back through the channel. The method includes a twelfth step of breaking away each guidance tab at the corresponding connection point and removing the guidance tabs through the channel. The method includes a final step of removing the cannula, suturing the incision, and bandaging the surgical site.

In a further embodiment, the instruments of the present disclosure include a hybrid washer tower for posterior surgical stabilization of a sacroiliac joint. The hybrid washer tower includes a columnar outer wall extending from a distal end to a proximal end. The columnar outer wall defines a hollow barrel extending from the distal end to the proximal end. The hollow barrel is at least partially enclosed by the outer wall. The hybrid washer tower also includes at least one lateral tang positioned on and projecting from a terminal portion of the distal end of the outer wall, so that it can engage with surrounding bone or tissue. Furthermore, the hybrid washer tower includes an interlock channel defined within the outer wall. The interlock channel has an open section positioned at a terminal portion of the proximal end of the outer wall to accept a connection pin positioned on a surgical instrument and direct the movement of the surgical instrument as it progresses through the hollow barrel.

In an additional embodiment, the present disclosure provides for a method for the posterior surgical stabilization of a sacroiliac joint using a hybrid washer tower. The method includes a first step of creating an incision proximal to a patient's sacroiliac joint to allow access to a posterior region of the sacroiliac joint. The method includes a second step of locating an implant site and introducing a guide pin to mark the implant site. The method includes a third step of fitting a dilator over the guide pin and dilating the incision. The method includes a fourth step of threading a hollow barrel of a hybrid washer tower over the dilator and progressing a distal end of the hybrid washer tower into the dilated incision. The method includes a fifth step of removing the dilator and the guide pin through the hollow barrel of the hybrid washer tower, leaving the hybrid washer tower positioned within the dilated incision to enable the passage of one or more surgical instruments and one or more implants through the hollow barrel to the implant site. The method includes a sixth step of inserting a drill through the hollow barrel until one or more blades of the drill are positioned proximate to the implant site, rotating the blades to create a void in the patient's sacroiliac joint, and removing the drill through the hollow barrel. The method includes a seventh step of coupling the implant to a distal end of an inserter, inserting the distal end of the inserter into the hollow barrel, orienting the inserter so that a connection pin positioned on a shaft of the inserter enters an interlock channel defined within an outer wall of the hybrid washer tower, and progressing the inserter through hollow barrel until the distal end of the inserter and the implant are positioned proximate to the void. The method includes an eighth step of rotating the inserter within the hollow barrel such that the rotation of the inserter affixes the implant within the sacroiliac joint, while simultaneously interlocking the inserter with hybrid washer tower. The method includes a ninth step of decoupling the implant from the inserter and applying a longitudinal force to a handle of the inserter to remove the interlocked inserter and hybrid washer tower from the incision simultaneously, without leaving any break away components. The method includes a final step of suturing and bandaging the incision.

Additional features and advantages of the technology described in this disclosure will be set forth in the detailed description which follows, and in part will be readily apparent to those skilled in the art from the description or recognized by practicing the technology as described in this disclosure, including the detailed description which follows, the claims, as well as the appended drawings.

### BRIEF DESCRIPTION OF THE DRAWINGS

The embodiments set forth in the drawings are illustrative and exemplary in nature and not intended to limit the disclosure. The following detailed description of the illustrative embodiments can be understood when read in conjunction with the following drawings, where like structure is indicated with like reference numerals and in which:

FIG. 1 is an anterior view of the bony anatomy of the pelvis and sacrum in accordance with various disclosed aspects herein;

FIG. 2 is a posterior view of the bony anatomy of the pelvis and sacrum in accordance with various disclosed aspects herein;

FIG. 3 is a right lateral view of the bony anatomy of the pelvis and sacrum in accordance with various disclosed aspects herein;

FIG. 4 is a left lateral view of the bony anatomy of the pelvis and sacrum in accordance with various disclosed aspects herein;

FIG. 5 is a superior view of the bony anatomy of the pelvis and sacrum in accordance with various disclosed aspects herein;

FIG. 6 is an inferior view of the bony anatomy of the pelvis and sacrum in accordance with various disclosed aspects herein;

FIG. 7 is an oblique view of the right sacroiliac joint in accordance with various disclosed aspects herein;

FIG. 8 is a perspective view of a joint probe in accordance with various disclosed aspects herein;

FIG. 9 is an enlarged view of the joint probe in FIG. 8 in accordance with various disclosed aspects herein;

FIG. 10 is an end view of the joint probe in FIG. 8 in accordance with various disclosed aspects herein;

FIG. 11 is a perspective view of a guide pin in accordance with various disclosed aspects herein;

FIG. 12 is an end view of the guide pin in FIG. 11 in accordance with various disclosed aspects herein;

FIG. 13 is a perspective view of a joint cutting instrument in accordance with various disclosed aspects herein;

FIG. 14 is an enlarged view of the joint cutting instrument in FIG. 13 in accordance with various disclosed aspects herein;

FIG. 15 is an end view of the joint cutting instrument in FIG. 13 in accordance with various disclosed aspects herein;

FIG. 16 is a perspective view of a t-handle addition for a joint cutting instrument in accordance with various disclosed aspects herein;

FIG. 17 is an end view of the t-handle addition in FIG. 16 in accordance with various disclosed aspects herein;

FIG. 18 is a perspective view of a joint cutting assembly in accordance with various disclosed aspects herein;

FIG. 19 is an exploded, perspective view of the joint cutting assembly in FIG. 18 in accordance with various disclosed aspects herein;

FIG. 20 is a perspective view of a fixation pin insertion assembly in accordance with various disclosed aspects herein;

FIG. 21 is an exploded, perspective view of the fixation pin insertion assembly in FIG. 20 in accordance with various disclosed aspects herein;

FIG. 22 is a side view of a box chisel in accordance with various disclosed aspects herein;

FIG. 23 is a side view of a rasp in accordance with various disclosed aspects herein;

FIG. 24 is a perspective view of a bone graft impactor in accordance with various disclosed aspects herein;

FIG. 25a is a perspective view of a fusion implant having helical fixation elements in accordance with various disclosed aspects herein;

FIG. 25b is a perspective view of a fusion implant having helical fixation elements in accordance with various disclosed aspects herein;

FIG. 25c is a top plan view of a fusion implant having helical fixation elements in accordance with various disclosed aspects herein;

FIG. 25d is a bottom plan view of a fusion implant having helical fixation elements in accordance with various disclosed aspects herein;

FIG. 26a is a perspective view of a fusion implant having helical fixation elements in accordance with various disclosed aspects herein;

FIG. 26b is a perspective view of a fusion implant having helical fixation elements in accordance with various disclosed aspects herein;

FIG. 26c is a bottom plan view of a fusion implant having helical fixation elements in accordance with various disclosed aspects herein;

FIG. 27a is a perspective view of a fusion implant having helical fixation elements in accordance with various disclosed aspects herein;

FIG. 27b is a side elevation view of a fusion implant having helical fixation elements in accordance with various disclosed aspects herein;

FIG. 27c is a bottom plan view of a fusion implant having helical fixation elements in accordance with various disclosed aspects herein;

FIG. 27d is a perspective view of a fusion implant having a helical fixation element in accordance with various disclosed aspects herein;

FIG. 27e is a side elevation view of a fusion implant having a helical fixation element in accordance with various disclosed aspects herein;

FIG. 27f is a bottom plan view of a fusion implant having a helical fixation element in accordance with various disclosed aspects herein;

FIG. 28a is a perspective view of a surgical tool in accordance with various disclosed aspects herein;

FIG. 28b is a side view of a surgical tool in accordance with various disclosed aspects herein;

FIG. 29a is a perspective view of a surgical tool in accordance with various disclosed aspects herein;

FIG. 29b is a perspective view of a surgical tool in accordance with various disclosed aspects herein;

FIG. 29c is a bottom plan view of a surgical tool in accordance with various disclosed aspects herein;

FIG. 29d is a perspective view of a surgical tool in accordance with various disclosed aspects herein;

FIG. 29e is a side view of a surgical tool in accordance with various disclosed aspects herein;

FIG. 30a is a perspective view of a surgical tool in accordance with various disclosed aspects herein;

FIG. 30b is a side elevation view of a surgical tool in accordance with various disclosed aspects herein;

FIG. 30c is a bottom plan view of a surgical tool in accordance with various disclosed aspects herein;

FIG. 31a is a perspective view of a surgical tool in accordance with various disclosed aspects herein;

FIG. 31b is a perspective bottom view of a surgical tool in accordance with various disclosed aspects herein;

FIG. 32a is a perspective view of a surgical tool in accordance with various disclosed aspects herein;

FIG. 32b is a perspective view of a surgical tool in accordance with various disclosed aspects herein;

FIG. 32c is a side elevation view of a surgical tool in accordance with various disclosed aspects herein;

FIG. 32d is a side elevation view of a surgical tool in accordance with various disclosed aspects herein;

FIG. 32e is a perspective view of a surgical tool in accordance with various disclosed aspects herein;

FIG. 33 is a perspective view of a surgical tool in accordance with various disclosed aspects herein;

FIG. 34a is a perspective view of a surgical tool in accordance with various disclosed aspects herein;

FIG. 34b is a perspective view of a surgical tool in accordance with various disclosed aspects herein;

FIG. 35a is a perspective view of a surgical tool in accordance with various disclosed aspects herein;

FIG. 35b is a perspective view of a surgical tool in accordance with various disclosed aspects herein;

FIG. 35c is a perspective view of a surgical tool in accordance with various disclosed aspects herein;

FIG. 36a illustrates a step in a surgical method in accordance with various disclosed aspects herein;

FIG. 36b illustrates a step in a surgical method in accordance with various disclosed aspects herein;

FIG. 36c illustrates a step in a surgical method in accordance with various disclosed aspects herein;

FIG. 36d illustrates a step in a surgical method in accordance with various disclosed aspects herein;

FIG. 36e illustrates a step in a surgical method in accordance with various disclosed aspects herein;

FIG. 36f illustrates a step in a surgical method in accordance with various disclosed aspects herein;

FIG. 36g illustrates a step in a surgical method in accordance with various disclosed aspects herein;

FIG. 36h illustrates a step in a surgical method in accordance with various disclosed aspects herein;

FIG. 36i illustrates a step in a surgical method in accordance with various disclosed aspects herein;

FIG. 36j illustrates a step in a surgical method in accordance with various disclosed aspects herein;

FIG. 36k illustrates a step in a surgical method in accordance with various disclosed aspects herein;

FIG. 36l illustrates a step in a surgical method in accordance with various disclosed aspects herein;

FIG. 37a illustrates a step in a surgical method in accordance with various disclosed aspects herein;

FIG. 37b illustrates a step in a surgical method in accordance with various disclosed aspects herein;

FIG. 37c illustrates a step in a surgical method in accordance with various disclosed aspects herein;

FIG. 37d illustrates a step in a surgical method in accordance with various disclosed aspects herein;

FIG. 37e illustrates a step in a surgical method in accordance with various disclosed aspects herein;

FIG. 37f illustrates a step in a surgical method in accordance with various disclosed aspects herein;

FIG. 37g illustrates a step in a surgical method in accordance with various disclosed aspects herein;

FIG. 37h illustrates a step in a surgical method in accordance with various disclosed aspects herein;

FIG. 37i illustrates a step in a surgical method in accordance with various disclosed aspects herein;

FIG. 37j illustrates a step in a surgical method in accordance with various disclosed aspects herein;

FIG. 37k illustrates a step in a surgical method in accordance with various disclosed aspects herein;

FIG. 37l illustrates a step in a surgical method in accordance with various disclosed aspects herein;

FIG. 37m illustrates a step in a surgical method in accordance with various disclosed aspects herein;

FIG. 37n illustrates a step in a surgical method in accordance with various disclosed aspects herein;

FIG. 37o is a front view of an example inserter for inserting a fusion implant in accordance with various disclosed aspects herein;

FIG. 37p is another front view of the example inserter of FIG. 370;

FIG. 38 is a transparent view of the pelvis to demonstrate the implantation of a fusion implant in the sacroiliac joint in accordance with various disclosed aspects herein;

FIG. 39 is a transparent view of the pelvis to demonstrate the implantation of a fusion implant in the sacroiliac joint in accordance with various disclosed aspects herein;

FIG. 40 is a transparent view of the pelvis to demonstrate the implantation of two fusion implants in the sacroiliac joint in accordance with various disclosed aspects herein;

FIG. 41 is a perspective view of a surgical tool in accordance with various disclosed aspects herein;

FIG. 42 is a perspective view of a surgical tool in accordance with various disclosed aspects herein;

FIG. 43a illustrates a step in a surgical method in accordance with various disclosed aspects herein;

FIG. 43b illustrates a step in a surgical method in accordance with various disclosed aspects herein;

FIG. 43c illustrates a step in a surgical method in accordance with various disclosed aspects herein;

FIG. 43d illustrates a step in a surgical method in accordance with various disclosed aspects herein;

FIG. 43e illustrates a step in a surgical method in accordance with various disclosed aspects herein;

FIG. 43f illustrates a step in a surgical method in accordance with various disclosed aspects herein;

FIG. 43g illustrates a step in a surgical method in accordance with various disclosed aspects herein;

FIG. 43h illustrates a step in a surgical method in accordance with various disclosed aspects herein;

FIG. 43i illustrates a step in a surgical method in accordance with various disclosed aspects herein;

FIG. 43j illustrates a step in a surgical method in accordance with various disclosed aspects herein;

FIG. 44a illustrates a perspective view of a surgical tool in accordance with various disclosed aspects herein;

FIG. 44b illustrates a perspective view of a surgical tool in accordance with various disclosed aspects herein;

Reference will now be made in greater detail to various embodiments of the present disclosure, some embodiments of which are illustrated in the accompanying drawings. Whenever possible, the same reference numerals will be used throughout the drawings to refer to the same or similar parts.

### DETAILED DESCRIPTION

The present disclosure relates to novel fusion implants 400 and surgical tools (and methods) designed for repairing a damaged or injured joint in a human patient, particularly a sacroiliac joint 102, and methods for using such fusion and/or fixation implants and tools in procedures for repairing the damaged or injured joint. More specifically, in aspects, the present disclosure pertains to a method for fixing and/or fusing a damaged joint using a fusion implant 400 having fixation element(s) (e.g., integrally formed helical fixation elements). In some embodiments, the fusion implants 400 of the present disclosure may be further used to compress the targeted joint. In aspects, the fusion implant 400 may include a gap, hole, or interior space operable to hold bone growth-promoting materials that provide for mechanical stability and promote the formation of a contiguous piece of bone between two bones in the targeted joint (e.g., the sacrum 101 and the ilium 100).

The fusion implants 400 may be applied to the joint using a novel surgical tool kit without the need for additional patient positioning or secondary surgery. The novel surgical tool kit may include a joint fusion implant 400 as described herein, a cannula 250 for exposing the sacroiliac joint 102 from a posterior approach, a washer 310 for insertion into the cannula 250 prior to the insertion of the fusion implant 400 into the sacroiliac joint 102 that includes a channel 256 and a driver 370 operable to be insert a joint fusion implant 400 into the sacroiliac joint 102, and an inserter 340 operable to insert the joint fusion implant 400 through the hollow space defined by the washer 310 into the sacroiliac joint 102 and implant 400 the fusion implant 400 into the articular surfaces of the ilium 100 and sacrum 101. The instrument kit may include additional surgical instruments for establishing an incision over the posterior aspect of the sacroiliac joint 102, such as a drill 260, sensor(s), guide wire 114, dilator 116, and other devices. It is noted, that in embodiments the dilator 116 and joint finder 350 may accomplish substantially similar effects and may be referred to interchangeably.

As used hereinafter, the terms “distal” and “proximal” are used to convey the relative position of various aspects of the present disclosure. The term “distal” or “distally” is used herein to refer to elements of actions that are positioned toward the implant site and away from the surgeon or medical personnel, as the element is used. The term “proximal or “proximally” is used herein to refer to elements of actions that are positioned away from the implant site and toward the surgeon or medical personnel, as the element is used.

Furthermore, as used hereinafter, the terms “longitudinal” and “transverse” are used to convey the relative direction of various aspects of the present disclosure. The term “longitudinal” is used herein to refer to elements that are oriented in-plane (i.e., parallel) with the implant direction. Whereas, the term “transverse” is used herein to refer to elements that are oriented out-of-plane (i.e., perpendicular) with the implant direction.

With respect to some embodiments, an approach is described to address the targeted joint through a posterior approach while delivering a fusion implant 400 device that may fix and facilitate fusion of the joint. The fusion implant 400 may also deliver bone growth-promoting material (e.g., autologous bone, allograft, BMP, etc.). The fusion implant 400 may be delivered to the joint, placed between bones in the targeted joint, and one or more fixation elements (e.g., integral fixation elements) of the fusion implant 400 may be engaged with bone tissue in the articular surfaces of adjacent bones in the targeted joint to thereby fix the targeted joint. Engagement with the bone tissue may also compress the targeted joint.

In aspects, the joint fusion implants 400 of the present disclosure may include fixation elements that may be helical anchors 403 positioned at or near a perimeter or circumference of the joint fusion implant 400. In such embodiments, the fixation elements are operable to be implanted in the tissue of the targeted joint, such as the cortical bone tissue, e.g., by rotation of the joint fusion implant 400 when it is positioned adjacent to the bones of the joint. An inserter 340 attached to the head 402 of the joint fusion implant 400 may advance the joint fusion implant 400 to the point that it is positioned at the bones of the targeted joint and may then be use to rotate the joint fusion implant 400 to drive distal cutting edge 405 of the fixation elements (e.g., helical anchors 403) of the joint fusion implant 400 into the tissue of the joint, such as cortical tissue of the bone.

An example surgical kit according to the present disclosure may include a washer 310 or hybrid washer tower 500 for establishing stable purchase in the targeted joint prior to insertion of joint fusion implant 400 in order to stabilize a cannula 250 through which the joint fusion implant 400 is inserted into the joint to effectively target the joint and to prevent rotation or slippage of the other surgical devices (e.g., the inserter 340) during the insertion and implantation of the joint fusion implant 400 into the joint tissue (e.g., cortical bone tissue) of the targeted joint. Significant forces may be applied to the inserter 340 in order to drive penetration of bone tissue by the bone-engaging (e.g., penetrating) ends of the fixation elements, which may cause torque to the joint and the attached surgical instrumentation that can cause destabilized movement of the surgical instruments. The establishment of the washer 310 of the present disclosure in the targeted joint may prevent or reduce the risk of instrument rotation, slippage, or other displacement during the implantation of the joint fusion implant 400 in the targeted joint. The washer 310, hybrid washer tower 500, and other instruments of the novel instrument kit of the present disclosure enables a minimally invasive surgical method for repairing a targeted joint (e.g., a sacroiliac joint 102) that results in a secure, consistent, and reliable fusion of the joint. The novel instrument kit enables the insertion of the joint fusion implant 400 into the joint while avoiding damage to the soft and connective tissues in and around the joint by closely controlling the placement of the fusion implant 400.

### Relevant Anatomy Description

The surgical instruments, implants, and methods of the present disclosure are generally applicable to fixation and fusion procedures for planar, synarthrotic, and amphiarthrotic joints. The presently disclosed surgical instruments, implants, and methods are particularly applicable to such joints in the spine and pelvis 201. In particular, the surgical instruments, implants, and methods disclosed herein are applicable to fixation and fusion of the sacroiliac joint 102.

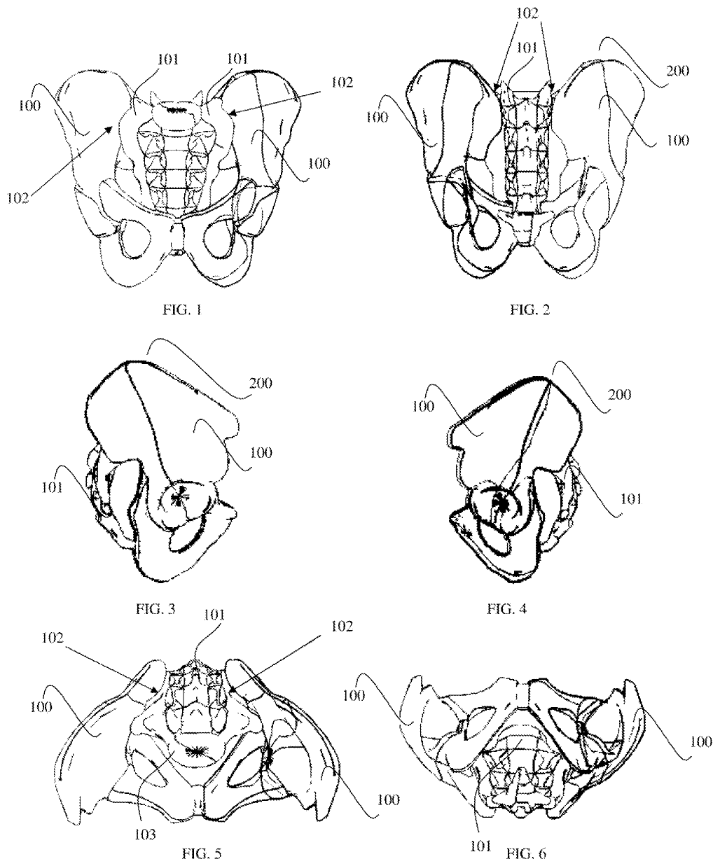

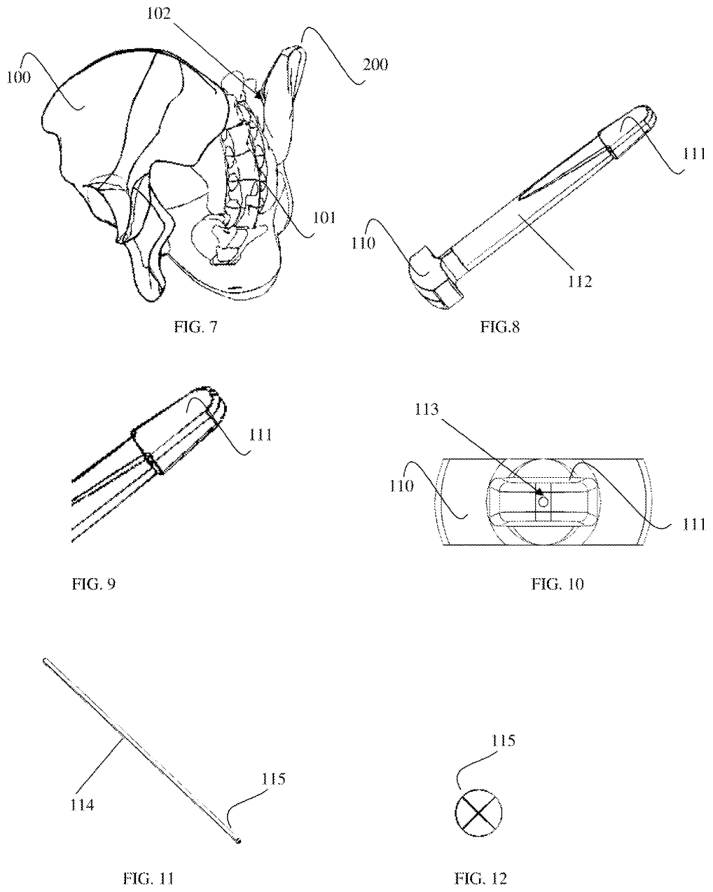

Referring to the drawings, FIG. 1 displays the bony anatomy of the sacrum 101 and pelvis 201 in a frontal, or anterior, view. The SI joint 102 is located between the ilium 100, or iliac wing 100, and the sacrum 101 at the base of the pelvis 201. Additionally, the ridgeline of the articular process, the lateral ala 203 and the pedicle 104 of vertebrae S1 can be observed in this view. In FIG. 2, it can be seen that the SI joint 102 is not fully exposed for direct visualization from a rear, or posterior, viewpoint due to the angled and raised iliac crest 200 of the iliac wing 100. This angle provides a landmark for the entry point of the exposure device of the present disclosure at the posterior iliac crest 200. In FIG. 3 and FIG. 4, the prominence of the iliac crest 200 is displayed, along with the large surface area of the iliac wing 100, while the SI joint 102 is fully enclosed between the iliac wing 100 and the sacrum 101 and occluded for direct visualization by the iliac wing 100. Again, the landmark of the posterior iliac crest 200 can be seen. From a top-down view, or superior view, the sacroiliac joint 102 can be fully observed between the iliac wing 100 and the sacrum 101, as shown in FIG. 5. Also shown in FIG. 5, the full sacrum 101 and specifically the vertebral body 103 of S1. The posterior superior iliac crest 200 and the entry point of the SI pedicle 104 can be observed in a direct line from one another.

In FIG. 6, it can be again observed that the SI joint 102 is occluded from direct visualization due to the anatomy of the sacrum 101, the ilium 100 and the pelvis 201. Therefore, the only direct visualization of the SI joint 102 can be achieved through an anterior, superior or posterior-oblique view of the sacrum 101 and pelvis 201. Due to major organs being present in the pelvic-sacral cavity (colon, rectum, bladder, etc.), an anterior or superior approach to the bony anatomy and, specifically the SI joint 102, presents an unreasonable risk. Lateral approaches can be performed as described in Published U.S. Pat. No. 5,334,205 to Cain, entitled “sacroiliac joint Fixation Guide,” incorporated herein by reference and Published U.S. Pat. No. 8,221,428 to Trieu, entitled “Sacro-iliac joint implant system, method and instrument,” incorporated herein by reference. However, these techniques rely on non-direct confirmation methods such as navigation and fluoroscopy to determine accurate landmark and sacroiliac joint 102 locations.

The present disclosure provides for novel surgical techniques, a novel joint fusion implant 400, and instrument designs which allow for a direct visualization of the SI joint 102 by utilizing a posterior-oblique access method to the anatomy as displayed in the oblique view of FIG. 7. In this drawing, the SI joint 102 can be clearly viewed between the right ilium 100 and sacrum 101. A corresponding joint may be exposed through the same approach on the left-hand side. Additionally, the anatomical landmark of the right posterior iliac crest 200 and the corresponding access to the S1 pedicle 104 can be seen through this approach.

### Instruments for Break-Away Method

The present disclosure utilizes a novel instrument kit that may be used in a surgical method to introduce and secure a fusion implant 400 into a targeted joint (e.g., an SI joint 102). The present disclosure also relates to novel implants, (e.g., fusion implants 400) that may be implanted into the SI joint 102, for example, by the novel surgical tools and methods of the present disclosure. Example instruments are described herein.

These surgical implements may be made of any suitable material, including, but not limited to, medical grade plastics, composites, metals, or alloys. In some embodiments, and without limitation, the tools are single use, in other embodiments the tools may be reused (and autoclaved, cleaned or otherwise suitably disinfected for further use). The tools may have various configurations, including those that differ from those depicted and specifically described herein.

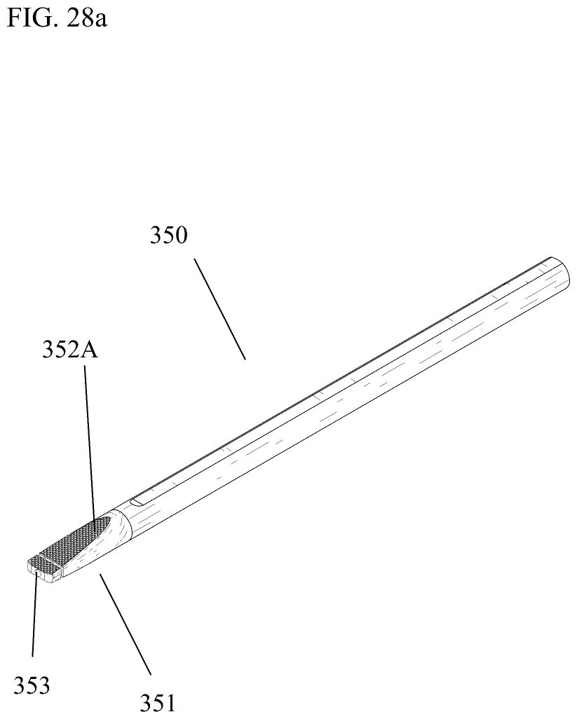

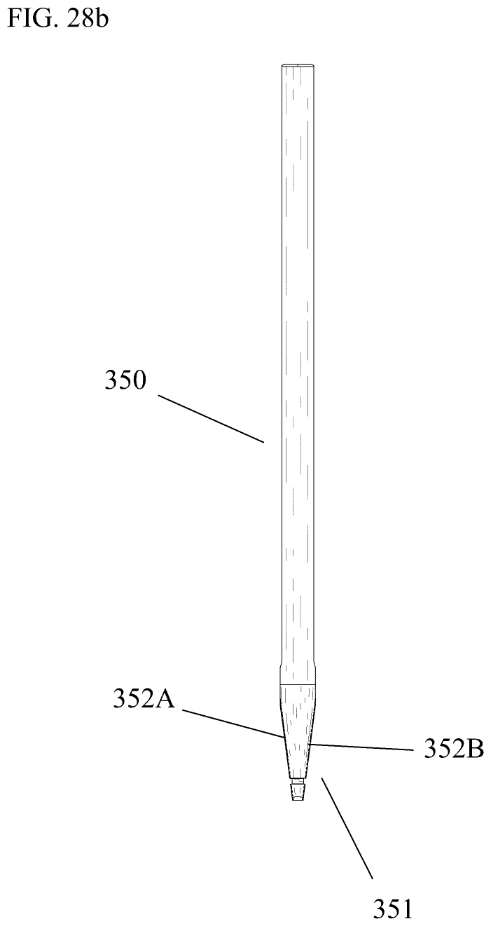

FIG. 28a-28b show a joint finder 350 that may be used to spread the joint and decorticate the articular surfaces of the joint in a single step. The joint finder 350 combines a head 351 having a wedge construction for placement between the articular surfaces of the targeted joint and rasping surfaces 352a and 352b that are operable to decorticate the articular surfaces of the targeted joint in preparation for the insertion of the tangs 312a, 312b of the washer 310 into the targeted joint space. The joint finder 350 may be passed into the incision over a guide wire 114 or guide pin 114 placed in the targeted joint threaded through the guide hole 353. An impact device (e.g., a mallet) may then be used to drive the joint finder 350 into the joint to distract the targeted joint and decorticate the targeted area of the targeted joint.

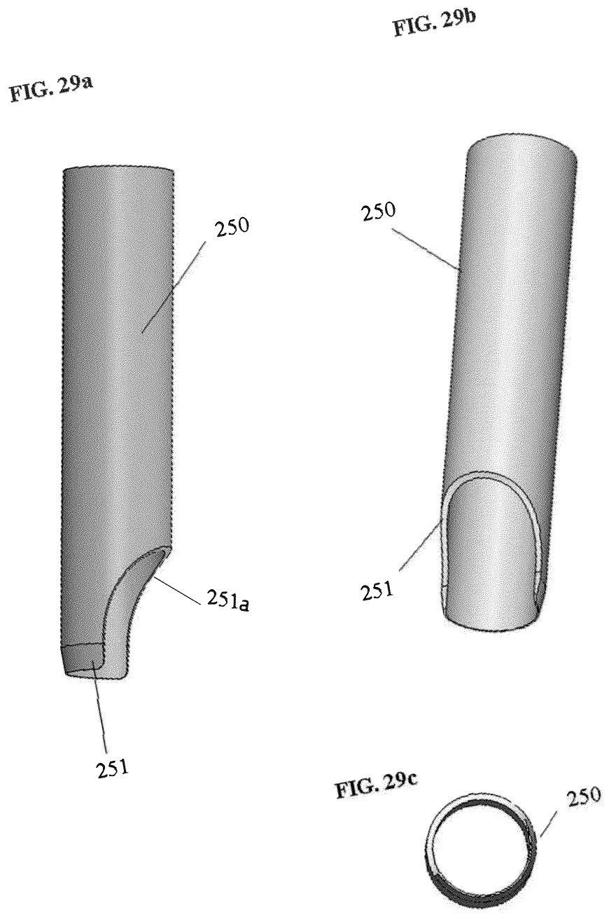

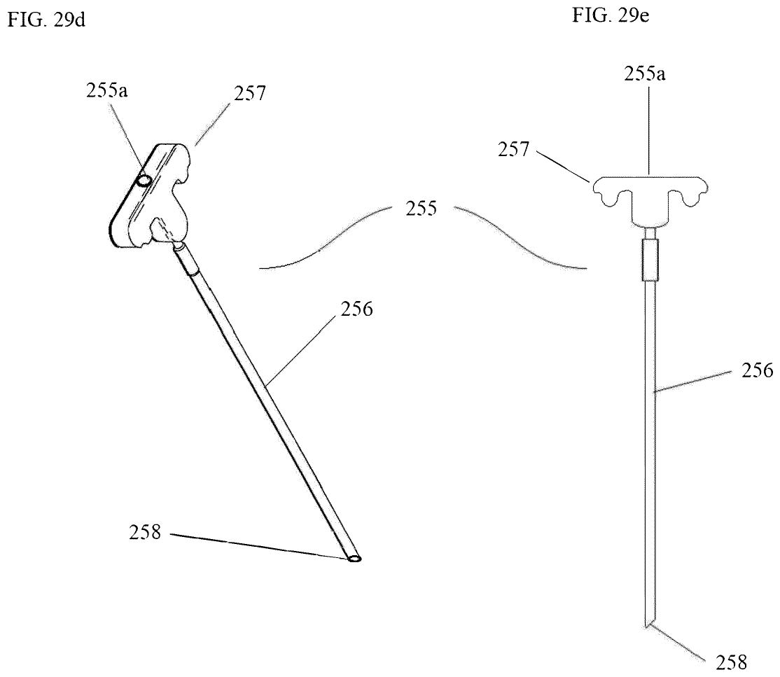

FIGS. 29a-29c show an example cannula 250 that may be engaged with a posterior side of the targeted joint (e.g., sacroiliac joint 102) after an incision has been made thereover. The cannula 250 may have a hollow channel 256 therein for passing various surgical tools that may have a cross-sectional shape corresponding to (e.g., complementary to) the channel 256. The channel 256 may provide a guide for inserting the various surgical tools and instruments into the targeted joint, allowing precise surgical incisions, insertions of the joint fusion implant 400, etc. The channel 256 of the cannula 250 may have an interior perimeter shape (i.e., a cross-sectional area) that is complementary to and/or accommodative of the perimeter shape of the washer 310 and the washer driver 330 of the present disclosure, allowing for a snug fit of the coupled washer 310 and washer driver 330 in the channel 256 of the cannula 250. The matching of the perimeter shapes of the interior of the barrel and the coupled washer 310 and washer driver 330 may allow the cannula 250 to be properly oriented over the joint when the joint fusion implant 400 is advanced into the targeted joint. For example, and without limitation, the example cannula 250 may have a channel 256 having a substantially circular interior cross-section that may accommodate a joint fusion implant 400 having circular cross-section (e.g., a helical implant 400) or a cross-section having a greatest diameter that is less than the diameter of the interior cross-section of the channel 256.

The channel 256 of the cannula 250 may also have guiding slots therein for properly aligning instruments (e.g., an inserter 340, an impactor 136, etc.). The guiding slots may engage notches or protrusions on the instruments such that the notches or protrusions slide along the guiding slots as the instrument or fusion implant 400 is advanced through the channel 256. Various implementations of the cannula 250 of the present disclosure may have one or more guiding slots (e.g., 1, 2, 3, etc.) and they may be arranged in various spatial arrangements on or within the channel 256.

The cannula 250 may have distal end 251 for positioning within the incision over the targeted joint. The distal end 251 may have a shape that is optimized for insertion into an incision over the targeted joint. The distal end 251 (i.e., the insertable end) may have a portion of its circumference removed to accommodate the bony protrusions and anatomy of the posterior pelvis 201 around the sacroiliac joint 102. For example, the distal end 251 of the cannula 250 may include a scoop 251a at the distal end 251 to accommodate adjacent bony protuberances, e.g., the posterior iliac crest 200. The distal end 251 may also have a tapered, rounded, and/or beveled edge that prevents or reduces damage to the soft and connective tissues in and around the incision of the targeted joint.

The cannula 250 may have other additional features such as attachment points for handle(s) (e.g., for inserting handle extensions, etc.), or integrally formed handles as well (not shown). Additionally, the attachment points or handle(s) may also be attachable to a stabilizing structure (e.g., a table or surgical arm, retractor/stabilizing arms, etc.) to aid in preventing movement of the exposure device or surgical implements engaged therewith during surgical procedure. It is to be appreciated that the above description of the exposure tool does not limit the present disclosure, and other features are contemplated in and within the scope of the present disclosure.

As shown in FIGS. 29d-29e, a first cannula 255 may be threaded over the guide pin 114 and inserted to the incision. The first cannula 255 may be utilized to confirm the position of the guide pin 114 in the joint space. The first cannula 255 may include a hollow channel 256 running from the proximal handle 257 to the distal opening 258. In some examples, the diameter of the channel 256 may be slightly larger than the diameter of the guide pin 114 to allow the first cannula 255 to have relatively small size, which allows for insertion into the joint space over the guide pin 114.

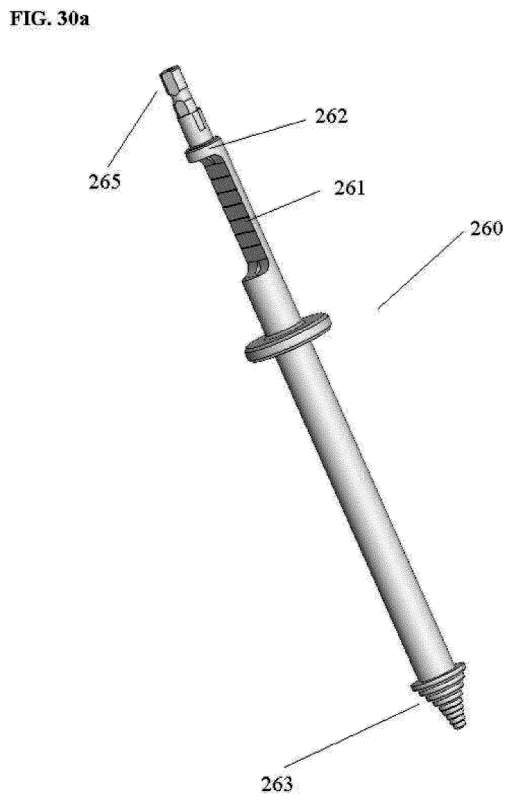

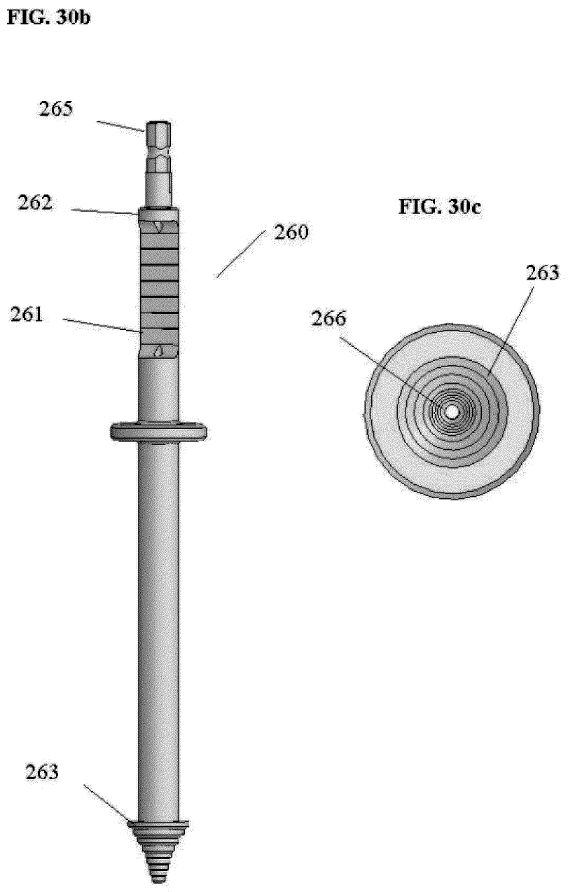

FIGS. 30a-30c show an example drill 260 used for bone preparation to enable fusion implant 400 insertion. In aspects, the drill 260 may be used for creating a void in the targeted joint for later insertion of a joint fusion implant 400. The drill 260 may have a recessed portion 261 along its proximal shaft that allows the medical personnel to monitor the depth to which the drill 260 penetrates the targeted joint. The shoulder 262 may be used as a marker in conjunction with the cannula 250. The shoulder 262 may have a pre-determined marking thereon, that when aligned with the upper rim of the cannula 250 indicates that the drill 260 has been advanced to desired depth in the targeted joint to provide a sufficient void for insertion of a joint fusion implant 400.

The drill 260 may have a threaded or stepped blade portion 263 with numerous concentric blades 263 in order to provide a void with the desired size in the targeted anatomy. The stepped blade 263 with the widest diameter may have a diameter that is equal to or substantially equal to the outer diameter of the washer 310 to allow for effective placement of the washer 310, nesting in the widest part of the void created by the drill 260 in the targeted joint.

The drill 260 may also be designed to be attached to power instruments, a hand drill or a handle 257. Without limiting the present disclosure, the example drill 260 may have a Jacob's chuck connection 265 so that it may be attached to a power tool for quick preparation. Additionally, the drill 260 may have a channel 266 running down its length that may allow the drill 260 to be passed into the joint over a guide wire 114 or guide pin 114 positioned in the incision.

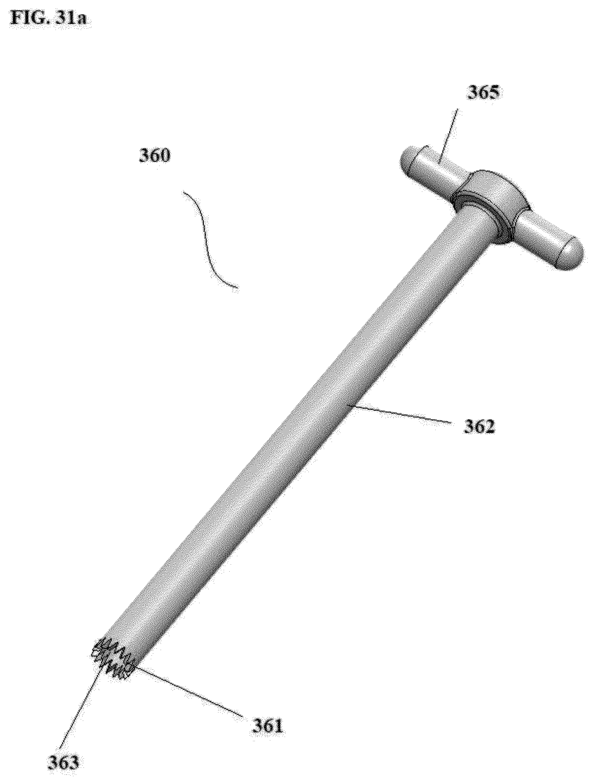

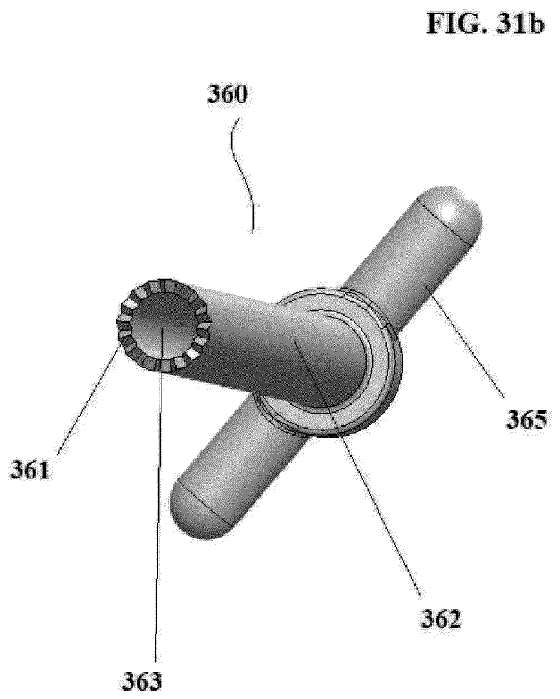

In some embodiments, the surgical kit may include a cylindrical drill 360 having a cylindrical blade 361, a drill shaft 362, and a handle 365 allowing for manual rotation of the cylindrical drill 360, as shown in FIG. 31a-31b. The cylindrical drill 360 may be passed over a dilator 116 or joint finder 350. For example, once the joint space is located with the joint finder 350 and the joint finder 350 is inserted into the joint space, the cylindrical drill 360 may be passed over the joint finder 350 with the head 351 of the joint finder 350 established in the joint space and placed in contact with the bone tissue of the targeted joint. The drill shaft 362 is hollow and has a sufficient diameter to receive the joint finder 350 and may have a sufficient length to reach the bone tissue in the targeted joint and rotate to create a void in the bone tissue to prepare the bone tissue for insertion of the joint fusion implant 400.

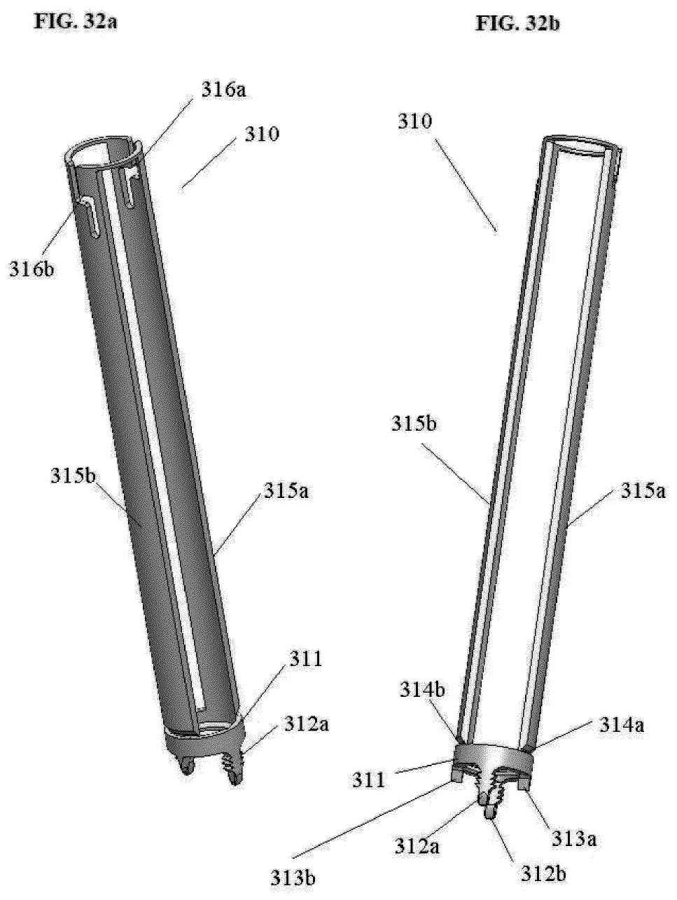

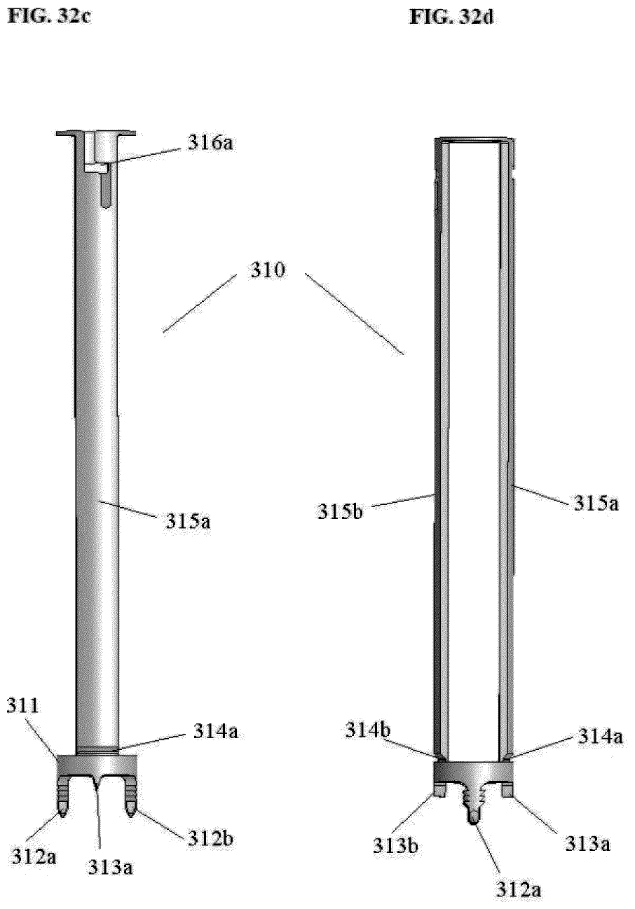

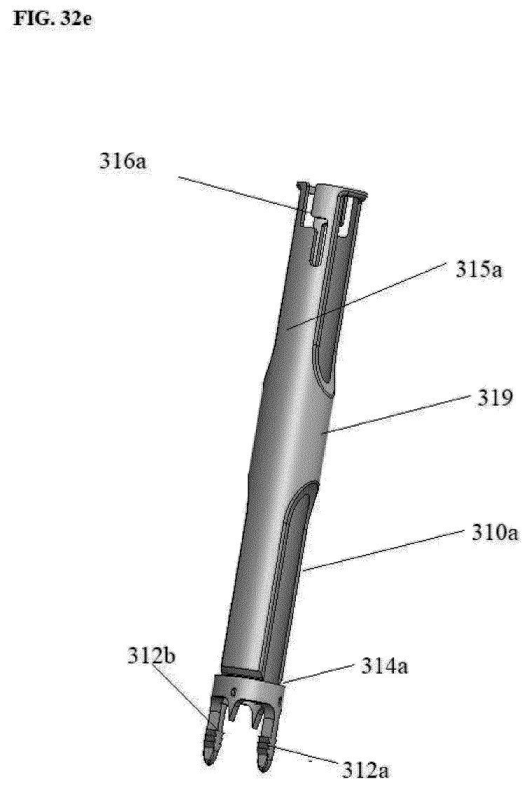

FIGS. 32a-32e, show an example embodiment of a washer 310 for insertion into a targeted joint (e.g., a sacroiliac joint 102). The washer 310 includes a washer ring 311 at its distal end that may include lateral tangs 312a, 312b and one or more protrusions 313a, 313b, extending distally from a first surface of the washer ring 311, that can penetrate tissues in the targeted joint and establish a stable purchase of the washer 310 before a joint fusion implant 400 is inserted and implanted into the targeted joint. The washer 310 may further include guidance tabs 315a, 315b that connect to the washer ring 311 at connection points 314a, 314b. The guidance tabs 315a, 315b may extend proximally from the second surface (i.e., the proximal surface) to form a semi-enclosed, columnar washer 310 structure with a hollow space disposed therein. As described herein, in embodiments, the washer ring 311 can be capable of breaking away or otherwise disconnecting from guidance tabs 315a, 315b. It is to be appreciated that this disengagement can result in disposable system or a reusable system, in accordance with the manner by which the disconnection is effected. The guidance tabs 315a, 315b may be operable to be engaged with a washer driver 330 in order to couple the washer 310 and the washer driver 330 together. The guidance tabs 315a, 315b may include coupling channels 316a, 316b that may be engaged with coupling pegs 331a, 331b of the washer driver 330. In other embodiments, the coupling channels 316a, 316b may be engaged with coupling pegs on any other applicable surgical instrument. The guidance tabs 315a, 315b may be operable to be removed from the washer ring 311 at connection points 314a, 314b. For example, the material of the guidance tabs 315a, 315b may be sufficiently thin and/or pre-treated (e.g., scored, thermally treated, perforated, intentionally weakened, chemically treated, or otherwise mechanically or materially prepared) such that the guidance tabs 315a, 315b may be disconnected, e.g., separated, mechanically disengaged, snapped or broken off at the connection points 314a, 314b such that they can be removed from the washer ring 311, leaving the washer ring 311 positioned at the targeted joint. In other examples, the guidance tabs 315a, 315b can be removed by other controlled disconnection techniques, e.g., by a shearing or cutting using a cutting tool, remote disengagement mechanisms. While many of the embodiments described herein would result in a disposable tool/device, it is to be understood and appreciated that yet other aspects exist that can separate the washer 310 while enabling reuse of the washer 310. For example, the connection points 314a, 314b may couple the washer ring 311 with the washer 310 using a selectively attachable fastening element. In embodiments, the selectively attachable fastening element may employ a delivery tool with a mechanically designed ball/detent-like system, a threaded assembly, magnets, latches or any other suitable mechanism that may enable reusable attachment between the washer ring 311 and the washer 310. Similarly, the selectively attachable fastening element may employ a delivery tool that separates a threaded, geometric or press fit washer 310 and may be reusable in later deployments in other surgeries, thereby enhancing efficiency and effectiveness of the present disclosure and claims appended hereto. In other words, it is to be appreciated that the novel concept of leaving the washer ring 311 in place in the body (separate or affixed to the implant 400) can be accomplished in a number of ways without departing from the spirit and/or scope of the present disclosure. Thus, these additional embodiments, together with those described herein, are to be included within the spirit and/or scope of the present disclosure and claims appended hereto.

In aspects, the lateral tangs 312a, 312b may be operable to penetrate tissue in the targeted joint by being driven into the joint, e.g., by impaction. While specific tang 312a, 312b shape(s) and/or size(s) is shown, it is to be understood and appreciated that the tangs 312a, 312b can deviate from those shown (e.g., in size, shape, number, etc.) while maintaining the functionality described herein. The lateral tangs 312a, 312b may include features that provide for structures that facilitate firm lodging in the tissue (e.g., connective tissues, cartilage, prepared bone surface, etc.). Such features may include barbs 313c (or other treatment(s)) 313c along the surface of the lateral tangs 312a, 312b. Such barbs 313c may have a retrograde angle (or other treatment) directed toward the proximal end of the washer 310 to prevent (or otherwise alleviate or mitigate) dislodgement of the washer 311 from the targeted joint. In addition to or in place of barbs 313c, the tangs 312a, 312b can be configured with other surface treatments that facilitate engagement, placement and/or retention of the tangs 312a, 312b. Similarly, other embodiments can be employed without such surface treatments as desired. In some examples, the lateral tangs 312a, 312b may be arranged such that they are diametrically or substantially diametrically positioned on washer ring 311 such that they can be driven between two adjacent articular surfaces of bones in the target joint, allowing penetration of the joint by the tangs 312a, 312b. In other examples, the washer 310 may include other arrangements of tangs 312a, 312b, such as a single tang 312a, or multiple tangs 312a, 312b arranged in various positions on the washer ring 311. The one or more protrusions 313a and 313b (or other surface treatment/design) may aid in establishing a stable purchase of the washer 310 in the targeted joint, e.g., providing penetration of tissues at edges of or adjacent to the targeted joint. While other treatments are suggested, examples can include, but are not limited to, knurling, shot peening, spray coatings, laser peening or etching, etc. These and other examples of effecting, creating and/or applying a surface treatment that assists in retention and/or attachment are to be considered within the scope of the disclosure and claims appended hereto.

FIG. 32e shows a further embodiment of a washer 310 of the present disclosure, washer 310a. The washer 310a shares most of the same features with the other examples of the washer 310, but has the added feature of bridges 319 extending between the guidance tabs 315a, 315b to form an integrated structure. The bridges 319 may provide additional structural stability to the washer 310a. To remove the guidance tabs 315a, 315b from washer 310a, both guidance tabs 315a, 315b may be pushed orthogonally to the washer ring 311 in the same direction and broken away at connection points 314a, 314b.

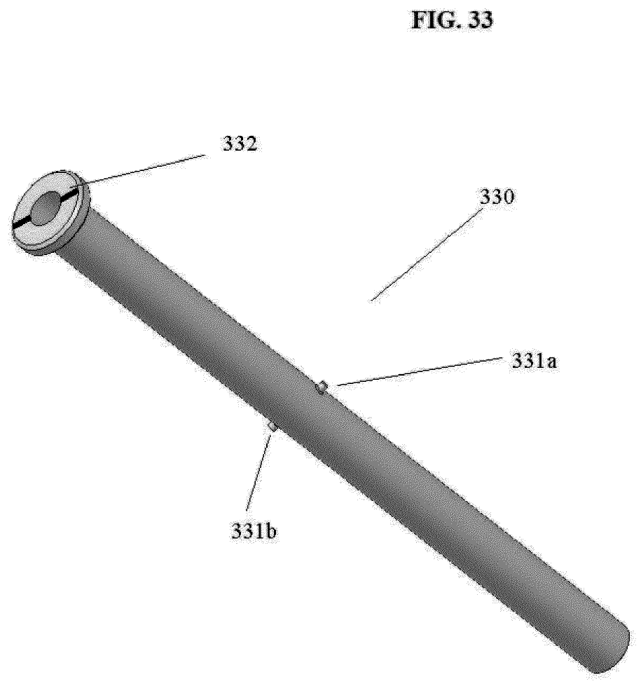

As shown in FIG. 33, the instrument kit of the present disclosure may also include a washer driver 330, which is operable to be coupled to the washer 310 to facilitate the insertion and penetration of the washer 310 into the targeted joint. In some examples, the washer driver 330 may have an external diameter that may fit within the inner diameter of the guidance tabs 315a, 315b of the washer 310 such that the guidance tabs 315a, 315b can be tightly fit over the washer driver 330. In such examples, the washer driver 330 may also include coupling pegs 331a, 331b which may be interlocked with coupling channels 316a, 316b of the guidance tabs 315a, 315b. The coupling pegs 331a, 331b may be slotted into the coupling channels 316a, 316b to reversibly fix the washer 310 and the washer driver 330 together. In embodiments, the coupling pegs 331a, 331b may interlock the washer 310 and washer driver 330 together when the washer driver 330 is rotated, twisted, or turned with respect to the washer 310. In embodiments, the size, orientation, and dimensions of the coupling channel 316a, 316b correspond to the depth and orientation of the implant site, enabling the coupling channel 316a, 316b to arrest the longitudinal movement of the coupling peg 331a, 331b to prevent the over insertion of a surgical instrument. In some embodiments, the coupling channels 316a, 316b may be configured to function and interact with the coupling pegs 331a, 331b according to the methods described hereinafter for the interlock channels 516. In other embodiments, the washer driver 330 and washer 310 may have complementary, nesting structure features that allow for a tight pressure fit between the washer 310 and the washer driver 330. For example, the washer driver 330 may have slots or cut-outs for accommodating the guidance tab(s) 315a, 315b of the washer 310 to establish a close fit between the guidance tab(s) 315a, 315b and the washer driver 330. Such slots or cut-outs may run longitudinally along the outer circumference of the washer driver 330 and have a complementary cross-sectional shape to that of the guidance tabs 315a, 315b. The washer driver 330 may also have additional structural features at its distal end that are operable to engage with the washer 310. For example, the distal end of the washer driver 330 may have a distal collar having a smaller outer diameter than the other portions of the washer driver 330, and the collar may be operable to nest within the inner diameter of the washer ring 311.

The washer driver 330 may have a length that is greater than the guidance tabs 315a, 315b, thereby allowing the washer driver 330 to be impacted at its proximal end with a mallet or other driving tool to advance the washer 310 into the targeted joint without damaging the guidance tabs 315a, 315b. The washer driver 330 may have blunt or flat end 332 that can be impacted with a driving tool such as a mallet. The washer driver 330 may be operable to be driven through the cannula 250 to in turn drive the lateral tangs 312a, 312b of the washer ring 311 into the targeted joint.

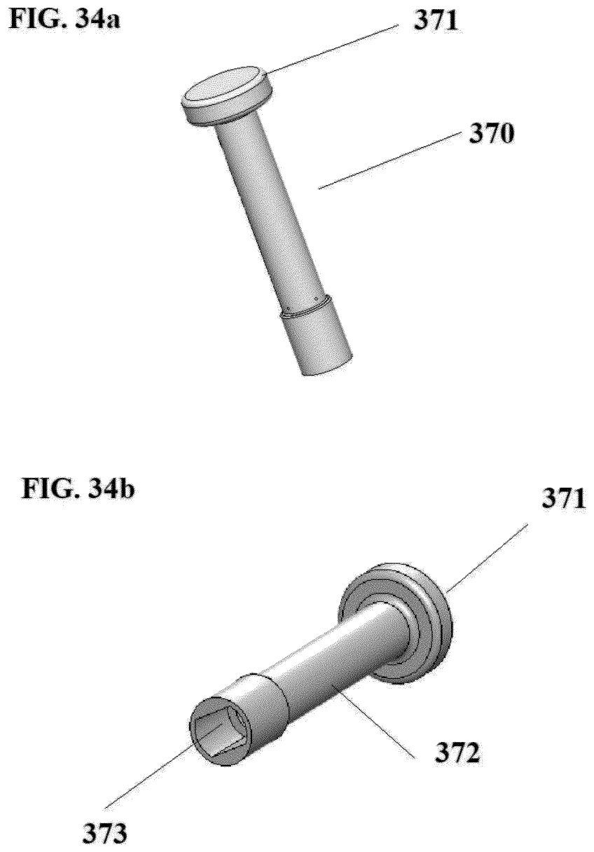

FIG. 34a-34b shows a driver 370 for driving one or more surgical instruments into a targeted joint (e.g., joint finder 350). The driver 370 has a head 371, a cylindrical shaft 372, and a canal 373 running through the shaft 372 for accepting surgical instruments to be driven into the targeted joint. The driver 370 may be passed over a dilator 116, joint finder 350, washer 310, or other surgical instrument such that the instrument can be driven into the targeted joint by striking the head 371 with a mallet or other appropriate device. The canal 373 may have a sufficient diameter to receive the surgical instruments (e.g., joint finder 350) to allow the driver 370 to drive the surgical instrument into the bone tissue. For example, once the joint space is located with the joint finder 350, the driver 370 may be passed over the joint finder 350. The head 371 of the driver 370 may then be struck to drive the joint finder 350 into the joint space.

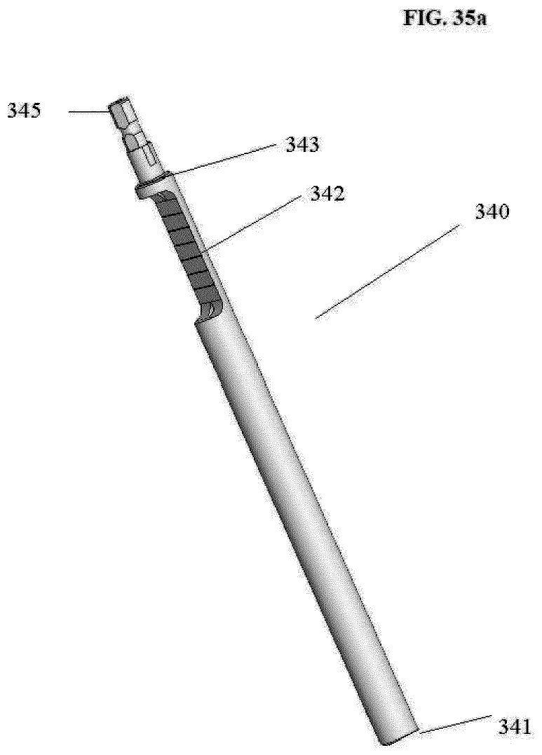

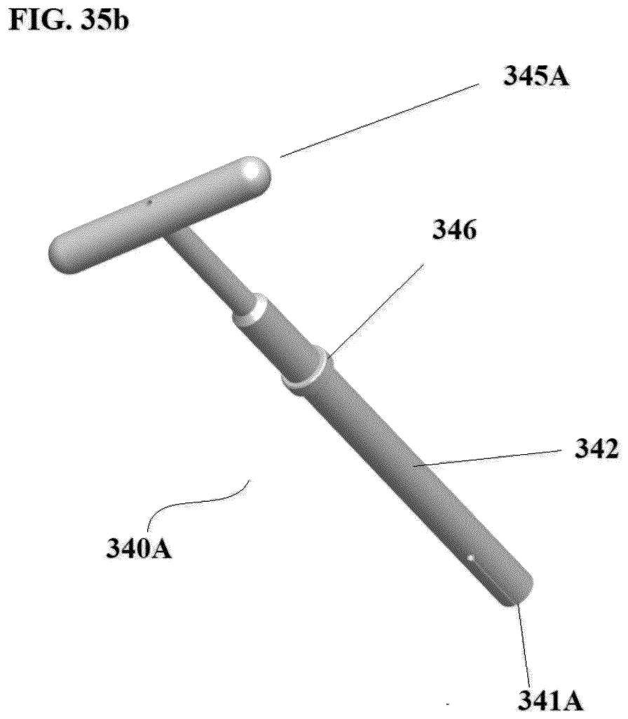

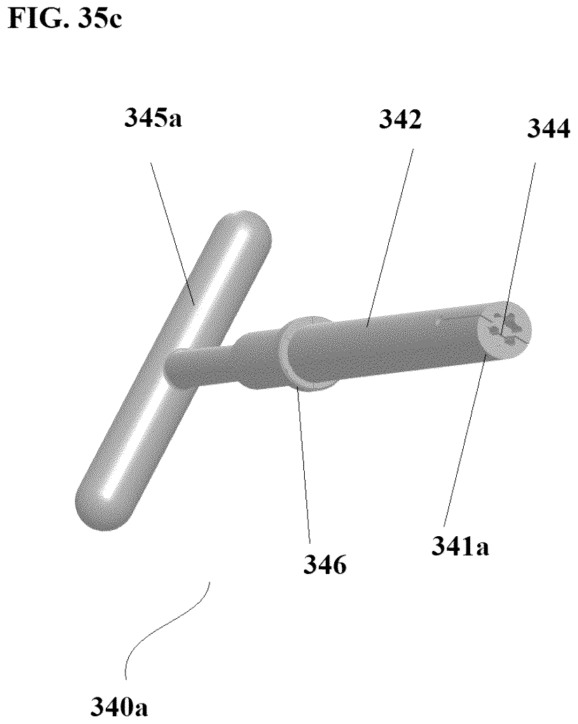

As shown in FIG. 35a, the instrument kit may also include an inserter 340 operable to engage with a joint fusion implant 400 and insert the joint fusion implant 400 into the targeted joint. Once the washer 310 is established in the targeted joint, the inserter 340 may be engaged with the head 402 (i.e., proximal end) of a joint fusion implant 400 and then used to advance the joint fusion implant 400 through the cannula 250 and into the targeted joint. The inserter 340 may have a distal end 341 configured to attach to the joint fusion implant 400, e.g., having a bit-receiving recess 344 (e.g., star, hex, or other functional shape) for receiving a complementary head 402 of a joint fusion implant 400. The inserter 340 may have a mechanism for tightening the engagement of the bit recess 344 with the head 402 of the joint fusion implant 400. For example, an actuator operable by the medical personnel may control a spring-operated element, e.g., an element in the bit recess 344 that reversibly applies pressure to the head 402 of the joint fusion implant 400 to maintain the head 402 in the bit recess 344.

The inserter 340 may have a recessed portion 342 along its proximal shaft having a cutaway 343 that allows the medical personnel to monitor the depth to which the inserter 340 penetrates the targeted joint (e.g., the distal end of the cutaway 343 can be used as a marker). The cutaway 343 may act as a marker to be used in conjunction with the markers on the guidance tabs 315a, 315b to determine when the inserter 340 has reached the desired depth to deliver a joint fusion implant 400 into the targeted joint. For example, at least one guidance tab 315a, 315b may be marked to indicate the point to which the distal end of the cutaway 343 of the inserter 340 should be advanced to completely insert the fusion implant 400 into the targeted joint. In other examples, at least one guidance tab 315a, 315b may include two marks, a proximal mark to indicate the point to which the distal end of the cutaway 343 should advance to place the distal cutting edge 405 of the fusion implant 400 at superficial aspect of the bones in the joint, and a distal mark to indicate the point to which the cutaway 343 of the inserter 340 should be advanced to completely insert the fusion implant 400 into the targeted joint.

The inserter 340 may be used to advance the joint fusion implant 400 to the washer ring 311 to place the joint fusion implant 400 adjacent and superficial to the targeted joint. The first proximal position marker on a guidance tab 315a of the washer 310 may indicate to the medical personnel that the joint fusion implant 400 is at the superficial aspect of the targeted joint when the cutaway 343 of the inserter 340 is aligned therewith. The medical personnel may then rotate the inserter 340 using a driver 370 to cause the at least one lateral fixation element of the joint fusion implant 400 to penetrate the bone tissue of the targeted joint. The inserter 340 may have a head 432 comprising a Jacob's chuck connection at its distal end so that it may be attached to a driver 370. The medical personnel may continue rotating the inserter 340 until the cutaway 343 is aligned with the second distal position marker on a guidance tab 315a of the washer 310, which indicates that the joint fusion implant 400 is fully implanted into the bone tissue.

FIGS. 35b-35c show another example of an inserter 340A operable to engage with and insert the joint fusion implant 400 into the targeted joint. The inserter 340A may have a distal end 341A configured to attach to the head 402 of the joint fusion implant 400, e.g., having a bit-receiving recess 344 (e.g., star, hex, or other functional shape) for receiving a complementary head 402 of a joint fusion implant 400. The inserter 340A may have a mechanism for tightening the engagement of the bit recess 344 with the head 402 of the joint fusion implant 400. For example, an actuator operable by the medical personnel may control a spring-operated element, e.g., an element in the bit recess 344 that reversibly applies pressure to the head 402 of the joint fusion implant 400 to maintain the head 402 in the bit recess 344.

Once the washer 310 is established in the targeted joint, the inserter 340A may be engaged with the head 402 of a joint fusion implant 400 and then used to advance the joint fusion implant 400 through the washer 310 and/or the cannula 250 and into the targeted joint. The inserter 340A may have a shaft 348 having sufficient length to allow the fusion implant 400 into the joint through the washer 310 and/or the cannula 250. The inserter 340A may include markings or notches on the shaft 348 that allows the medical personnel to monitor the depth to which the fusion implant 400 held by the inserter 340A penetrates the targeted joint. The radial ledge 346 may be used in conjunction with the guidance tabs 315a, 315b to determine when the inserter 340A has reached the desired depth to deliver a joint fusion implant 400 into the targeted joint. For example, a radial ledge 346 may indicate the point to which the inserter 340A should be advanced to completely insert the fusion implant 400 into the targeted joint when the radial ledge 346 abuts the top of the guidance tabs 315a, 315b.

The inserter 340A may be used to advance the joint fusion implant 400 to the washer ring 311 to place the joint fusion implant 400 adjacent and superficial to the targeted joint. The inserter 340A may be rotated using handle 345A to cause the at least one lateral fixation element of the joint fusion implant 400 to penetrate the bone tissue of the targeted joint. The medical personnel may continue rotating the inserter 340A until the radial ledge 346 abuts the guidance tabs 315a, 315b of the washer 310, which indicates that the joint fusion implant 400 is fully implanted into the bone tissue. In other embodiments, the rotation of the inserter 340 may cause connection pins 518 to interlock with a channel 516 positioned on a hybrid washer tower 500 or any other washer 310 structure. The interlocked connection pin 518 and channel 516 may allow the inserter 340 and the hybrid washer tower 500 to move in unison when acted upon by a longitudinal force.

FIGS. 44a-44b show yet another embodiment of an inserter 340B. The inserter 340B may have a shaft 348B and a distal end 341B configured to attach to the head 402 of the joint fusion implant 400. The inserter 340B may include threads 349B or other fixation elements positioned at the proximal end of the shaft 340B. The threads 349B may be configured to enable the shaft 348B of the inserter 340B to couple with a threaded nut 320 positioned at a proximal end of the washer 310. As the inserter 340B is rotated to affix the implant 400 at the implant site, the threads 349B may couple with the threaded nut 320 to affix the inserter 340B with the proximal end of the washer 310. In embodiments, this mechanism may allow a medical professional to retract the inserter 340B and the removable components of the washer 310 (i.e., the threaded nut 320 and the guidance tabs 315a, 315b), which may simultaneously detach the guidance tabs 315a, 315B from their connection points 314a, 314B, leaving the washer ring 311 in place. The interaction between the threaded nut 320 and the inserter 340B may have additional benefits beyond merely coupling the threaded nut 320 and the inserter 340B. In embodiments, the threaded nut 320 may provide additional mechanical stability to the guidance tabs 315a, 315b at their proximal end. Furthermore, the threads 349B and the threaded nut 320 may be positioned at a predetermined location relative to the implant site to serve as a depth/insertion control guide, preventing over insertion of the inserter 340B into the SI joint 102. In embodiments, the threads 349B may be either female or male threads and the threaded nut 320 may have opposite, corresponding threading to couple with the threads 349B.

Various additional instruments may be included in such a set, including a joint cutting instrument (e.g., dilator 116), guide pins 114, guide pin assemblies 114, a rasp 132, a box chisel 131, an inserter 340, an impactor 136, and other instruments. Each of such tools may correspond to the exposure devices described herein. For example, the joint cutting instrument, the rasp 132, the box chisel 131, and the impactor 136 each may have a shape that is complementary to the channel 256 of the cannula 250, allowing each instrument to be inserted into the channel 256 and in the proper orientation, without room to deviate from the path of the channel 256.

The implements may include a joint probe 112 capable of being used to locate an insertion point in a targeted joint for a fusion implant 400. The joint probe 112 may have a hollow aperture 113 therethrough for inserting a guide wire 114 into the SI joint 102 once the joint probe 112 is properly positioned in the insertion point. Referring to FIGS. 8-11, the joint probe 112 may have a rounded tip 111 for locating the insertion point, a shaft, and a handle 110. A hollow aperture 113 runs through the length of the joint probe 112 to allow a guide wire 114 to be inserted therethrough and into the targeted joint.

The set of tools may also include guide pins 114 for securing the exposure device to the SI joint 102 and the ilium 100. Referring to FIGS. 11-12, the guide pin 114 may have shaft that corresponds to the hollow aperture 113 of the joint probe 112 and may be inserted into the targeted joint through the dilator 116, to guide tools and implements subsequently positioned in the targeted joint.

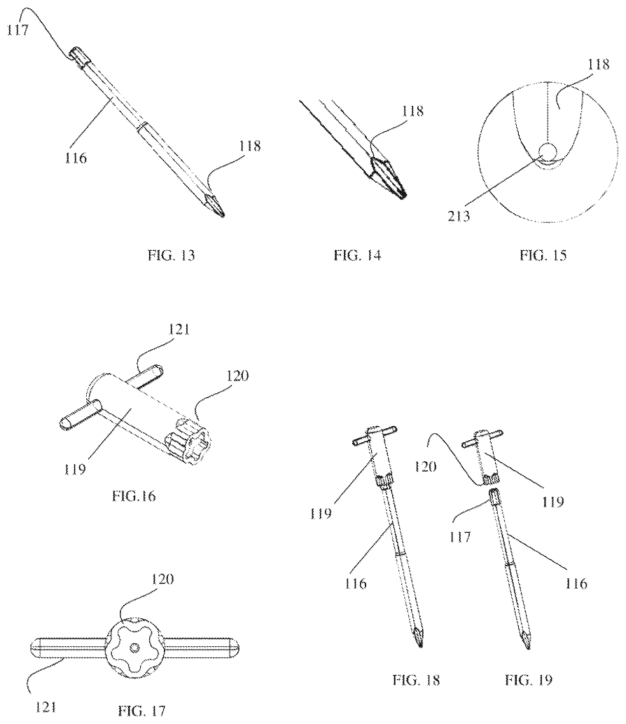

The set of surgical implements may include one or more dilators 116, which may be any device or structure capable of dilating an incision made in a human or other animal. FIGS. 13-19 illustrate an example of a dilator 116 system that may be included in the present disclosure. The dilators 116 may be made of any suitable material and may have any suitable dimensions and configuration. In embodiments, the dilator 116 may have a distal end 118, a proximal end 117, and a shaft there between. The distal end 118 may have any configuration suitable to dilate an opening or incision, for example an incision made by a guide pin 114 or guide wire 114 in the patient's flesh and dilate that incision to increase its size. The distal end 118 may be tapered, coming to a point at its end. The proximal end 117 may be faceted, allowing it to be engaged with a grooved receiver 120 of a T-handle 119. A dilator 116 assembly, an example of which is shown in FIGS. 18 and 19, allows the dilator 116 to be spun or otherwise manipulated to adjust the size of an incision. The dilator 116 (and the T-handle 119) may have a hollow column 213 running down its length that may allow a guide wire 114 or guide pin 114 to be inserted therethrough into the incision.

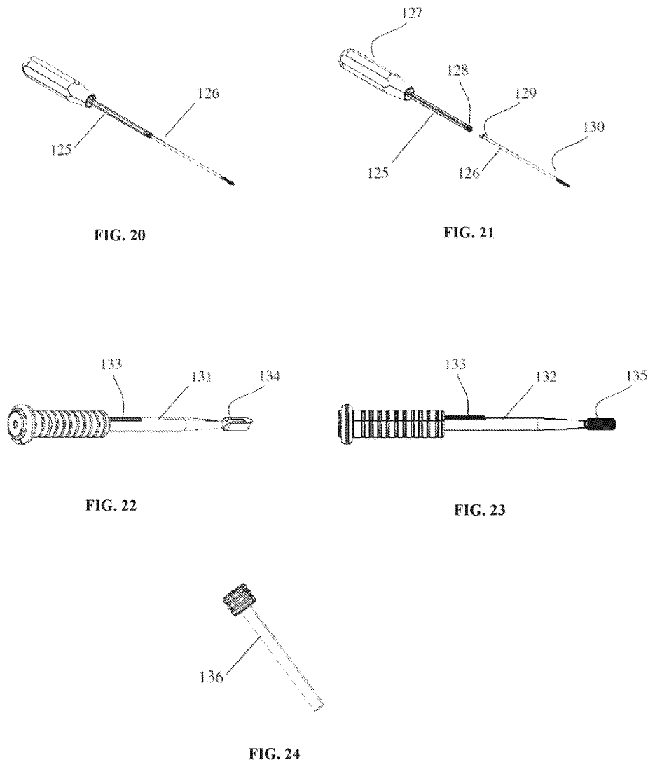

Fixing pins 126 and a fixing pin handle 125, as shown in FIGS. 20-21, may be included in the set of instruments that correspond to a guide slots in other instruments. For example, in some embodiments, the cannula 250 may include guide slots through which a fixing pin 126 or guide pin 114 may be inserted. The guide pins 114 or fixing pins 126 may also have a sharp and/or threaded end 115, 130 for piercing bone and other tissues. The fixing pins 126 can be used to secure the cannula 250 or other instruments in a desired position over the SI joint 102 or the ilium 100.

A chisel 131, as shown in FIGS. 22-23, may be included in the set to provide a tool for creating and/or widening an opening in the SI joint 102 with desired dimensions on its distal tip 134 to better facilitate entry for the joint fusion implant 400. The distal tip 134 may have a tapered nose in order to wedge its way in between the articular surfaces of bones in the targeted joint (e.g., the sacrum 101 and ilium 100). The distal tip 134 may also have cutting edges to dig into the bone and remove it from the surgical site. The chisel 131 may be used or otherwise used to aid in penetrating the cortical tissue of the sacrum 101 and ilium 100 to allow one or more helical anchors 403, 404 of the joint fusion implants 400 of the present disclosure to penetrate the bone tissue and thereby anchor the joint fusion implant 400 in the joint. The distal tip 134 may also have a containment device for removal of surgical site bone. A window, hole, or opening may be defined within the distal tip 134 to collect bone debris and remove the bone debris after removal from surgical site. The distal tip 134 may be undersized to a joint fusion implant 400 to be inserted in order to ensure full bony contact on all sides of the fusion site. The chisel 131 may have an outer diameter, as seen in FIG. 22 that matches the inner diameter of the hollow channel 256 of the cannula 250 or hybrid washer tower 500 to keep the chisel 131 directed in an axial plane for desired implant 400 preparation. The chisel 131 may have a timing and/or orientation feature 133 operable to mate with a female timing and/or orientation feature 133 on the inside of the cannula 250 or other incision exposure instrument to keep the chisel 131 from plunging too far into the surgical site and to further keep the chisel 131 in the proper orientation for desired implant site preparation.

Additionally, a rasp 132, as shown in FIG. 23, may be included in the set to provide a tool for conditioning, decorticating, and/or removing bone tissue in an opening in the targeted joint with desired dimensions on its edge 135 to better prepare the bone tissue in the targeted joint for a fusion site for the joint fusion implant 400. The edge 135 may have a tapered nose in order to wedge its way in between the articular surfaces of bones in the targeted joint (e.g., sacrum 101 and ilium 100). The edge 135 may have aggressive teeth which can scrape the bone to help prepare the bone surface (e.g., decorticate the surface) for receiving helical anchors 403, 404 of joint fusion implants 400 of the present disclosure. The rasp 132 may have a cross-sectional dimension as seen in FIG. 23, that is equal to or less than the inner diameter of the channel 256 of the cannula 250 or hybrid washer tower 500 to keep the rasp 132 directed in an axial plane for desired implant preparation. The rasp 132 may have a timing and/or orientation feature 133 that mates with a female timing and/or orientation feature 133 on the inside of the cannula 250 or other incision exposure instrument to keep the instrument to keep the instrument in the proper orientation for desired implant site preparation. In some embodiments, the timing and/or orientation feature 133 prevents the instrument from traveling too far into the surgical site.

One or more impactors, such as impactor 136 shown in FIG. 24, may be included in the surgical implements, as well. The impactor 136 may have a cylindrical proximal end, like a hammer. The impactor 136 may be included in the set to facilitate secondary impaction and movement of other surgical instruments, and, optionally, to advance bone-fusion promoting materials in the targeted joint in front of and/or behind the fusion implant 400 to better prepare the surgical site and to promote fusion of the targeted joint. The impactor 136 may have an outer diameter, as seen in FIG. 22, that matches the inner diameter of the cannula 250 or other incision exposure instrument to keep the instrument directed in an axial plane for desired surgical site preparation.

### Instruments for Interlock Method

The present disclosure utilizes a novel instrument kit that may be used in a novel surgical method to introduce and secure a fusion implant 400 into a targeted joint (e.g., an SI joint 102). The present disclosure also relates to novel implants, (e.g., fusion implants 400) that may be implanted into the SI joint 102, for example, by the novel surgical tools and methods of the present disclosure. Example instruments are described herein.

These surgical implements may be made of any suitable material, including, but not limited to, medical grade plastics, composites, metals, or alloys. In some embodiments, and without limitation, the tools are single use, in other embodiments the tools may be reused (and autoclaved, cleaned or otherwise suitably disinfected for further use). The tools may have various configurations, including those that differ from those depicted and specifically described herein.

In embodiments, some or all of the instruments discussed hereinabove may be used in conjunction with one or more additional instruments or alternative instrument embodiments to enable a medical professional to perform a posterior insertion of a fusion implant 400 into an SI joint 102 according to the interlock method discussed herein.

FIGS. 8-24 show tools that may be used to establish an incision and assist in establishing a void in the sacroiliac joint 102. A joint probe 112 may be used to identify the insertion area on the posterior side of the SI joint 102. The area of the SI joint 102 may be probed until the rounded geometry of the joint probe 112 finds or drops into the proper position in the SI joint 102, where an incision may be properly made. Subsequently, a guide pin 114 or guide wire 114 may be inserted through an aperture 113 in the joint probe 112 and into the patient to create an incision in the SI joint 102. Alternatively, the incision may be made by any suitable method, including scalpel or other cutting or dissection tool. The incision may be made proximal to the patient's SI joint 102, allowing the joint to be accessed by the exposure device. The guide pin 114 may be advanced until its distal end 115 is in contact with the SI joint 102 or at least partially within SI joint 102.

As shown in FIGS. 11-19, a dilator 116 may be used to dilate the incision. As an example, the dilator 116 may be slotted over the guide pin 114 through a column 213 running the length of the dilator 116. The tapered distal end 118 (i.e., distal end 118) of the dilator 116 may be slotted over the guide pin 114, and dilator 116 may then be advanced along the guide pin 114 to or near the SI joint 102 through the incision. As dilator 116 enters the incision, the tapered distal end 118 pushes the patient's tissue aside, thereby dilating the incision to accommodate an exposure device as described herein. A joint cutting assembly that includes the dilator 116 and a T-handle 119 engaged with a proximal end 117 of the dilator 116 may be used to further drive the dilator 116 into the incision to a desired depth to sufficiently expose the SI joint 102. Alternatively, an impactor 136 may be used to further drive the dilator 116 into the incision to a desired depth.

In embodiments, as shown in FIG. 28a-28b, a joint finder 350 may be used, in addition to or instead of the dilator 116, to spread the joint and decorticate the articular surfaces of the joint in a single step. In embodiments, a single tool may accomplish the effects of a dilator 116 and a joint finder 350. The joint finder 350 that combines a head 351 having a wedge construction for placement between the articular surfaces of targeted joint and rasping surfaces 352a and 352b that are operable to decorticate the articular surfaces of the targeted joint. The joint finder 350 may be passed into the incision over the guide pin 114 placed in the targeted joint threaded through the guide hole 353. An impact device (e.g., a mallet) may then be used to drive the joint finder 350 into the joint to distract the targeted joint and decorticate the targeted area of the targeted joint.

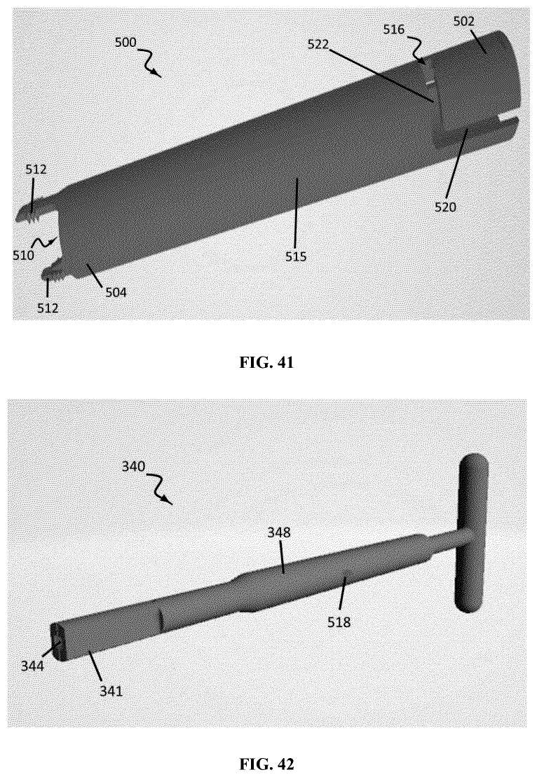

According to some embodiments, surgical tools and instruments may be used to enable an interlock method that can provide a medical professional access to the implant site when performing a posterior insertion of a fusion implant 400 into an SI joint 102, while also allowing for the efficient removal of surgical instruments and tools without any break-away components. To enable this method, a hybrid washer tower 500 may be used according to embodiments discussed herein. As shown in FIG. 41, the hybrid washer tower 500 may be used to achieve the individual functions of the cannula 250 and the washer 310 discussed herein. The hybrid washer tower 500 may include a columnar outer wall 515 with a proximal end 502 and a distal end 504. In embodiments, the distal end 504 may be shaped to be positioned within the incision over the targeted joint. The distal end 504 may have a shape that is optimized for insertion into an incision over the targeted joint. The distal end 504 may have a portion of its circumference removed to accommodate the bony protrusions and anatomy of the posterior pelvis 201 around the sacroiliac joint 102. For example, the distal end 504 of the hybrid washer tower 500 may have a cutaway portion or may be tapered, rounded, and/or have a beveled edge that prevents or reduces damage to the soft and connective tissues in and around the incision of the targeted joint

The outer wall 515 of the hybrid washer tower 500 may define a hollow barrel 510 extending from the proximal end 502 to the distal end 504. The hollow barrel 510 may be either completely or partially enclosed by the outer wall 515. The outer wall 515 may have a smooth surface on both its interior and exterior faces to minimally restrict instruments travelling through the hollow barrel 510 and to avoid unnecessary abrasive contact with tissue surrounding the implant site. Furthermore, in embodiments, the outer wall 515 may include one or more holes, recesses, apertures, notches, or other equivalent means that may permit a medical professional to monitor the position of an instrument as it passes through the hollow barrel 510 of the hybrid washer tower 500, according to methods discussed herein. The cross-section of the hollow barrel 510 may be a substantially circular interior cross-section that may accommodate a joint fusion implant 400 having circular cross-section (e.g., a helical implant 400) or any cross-section having a greatest diameter that is less than the diameter of the interior cross-section of the hollow barrel 510. Furthermore, in embodiments, the outer wall 515 may include one or more holes, recesses, apertures, notches, or other equivalent means that may permit a medical professional to monitor the position or depth of an instrument as it passes through the hollow barrel 510 of the hybrid washer tower 500, according to methods discussed herein.

After creation of the incision, the hollow barrel 510 of the hybrid washer tower 500 may be fit over the dilator 116 or joint finder 350 and advanced longitudinally until the distal end 504 of the hybrid washer tower 500 is positioned within the incision surrounding the implant site. Following the placement of the hybrid washer tower 500, the guide pin 114 and the dilator 116 or joint finder 350 have been removed through the hollow barrel 510 of the hybrid washer tower 500 and a driver 370 has been coupled to the proximal end 502 of the hybrid washer tower 500 to permit a medical professional to strike, drive, or otherwise advance the hybrid washer tower 500 longitudinally within the incision until the distal end 504 of the hybrid washer tower 500 is proximate to the implant site. In embodiments, the hybrid washer tower 500 may include one or more lateral tangs 512 extending from a terminal portion of the distal end 504 that can engage the surrounding bone or tissue around the implant site to maintain the hybrid washer tower 500 in a stationary position during a surgical implant procedure. The lateral tangs 512 may include barbs, hooks, threads, splines, or other fastening mechanisms positioned on a distal edge to engage with the tissue surrounding the implant site and facilitate firm lodging in the tissue. In embodiments, the lateral tangs 512 may further include other surface features on one or more exterior surfaces of the tangs 512 that facilitate engagement, placement and/or retention of the tangs 512. The barbs, hooks, threads, splines, or other fastening mechanisms may have a retrograde angle (or other treatment) directed toward the proximal end 502 of the hybrid washer tower 500 to prevent (or otherwise alleviate or mitigate) dislodgement of the hybrid washer tower 500 from the targeted joint. In aspects, the lateral tangs 512 may be operable to penetrate tissue in the targeted joint by being driven into the joint, e.g., by impaction. While specific tang 512 shape(s) and/or size(s) is shown, it is to be understood and appreciated that the tangs 512 can deviate from those shown (e.g., in size, shape, number, etc.) while maintaining the functionality described herein. In some examples, the lateral tangs 512 may be arranged such that they are diametrically or substantially diametrically positioned on distal end 504 of the hybrid washer tower 500 such that they can be driven between two adjacent articular surfaces of bones in the target joint, allowing penetration of the joint by the tangs 512. In other examples, the hybrid washer tower 500 may include other arrangements of tangs 512, such as a single tang 512, or multiple tangs 512 arranged in various positions on the distal end 504 of the hybrid washer tower 500. These and other examples of effecting, creating and/or applying a surface treatment that assists in retention and/or attachment are to be considered within the scope of the disclosure and claims appended hereto.

Additionally, the hybrid washer tower 500 may further include one or more interlock channels 516 defined within the outer wall 515. The interlock channel 516 may be an L-shaped recessed portion or an L-shaped open channel positioned at or near a proximal end 502 of the hybrid washer tower 500. In some embodiments, the interlock channel 516 may be positioned in the center or near the distal end 504 of the hybrid washer tower 500. The interlock channel 516 may be positioned at a terminal portion of the proximal end 502 of the outer wall 515 so that an open section of the interlock channel 516 may engage a connection pin 518. The interlock channel 516, may be comprised of a longitudinal portion 520 and a transverse portion 522. The longitudinal portion 520 and the transverse portion 522 may integrally form a single recess or channel, but may be separated by a right angle to direct, restrict, and control the movement of a connection pin 518, and thus a surgical instrument, within the interlock channel 516. It should be understood that the hybrid washer tower 500 may have any suitable cross-sectional geometry to enable the transport of various surgical instruments and implants discussed herein. Furthermore, the hybrid washer tower 500 may have any suitable dimensions, including the height and diameter of the hollow barrel 510, to facilitate the methods discussed hereinafter.

As shown in FIGS. 34a-34b, the driver 370 may have a head 371, a cylindrical shaft 372, and a canal 373 running through the shaft 372 for accepting a portion of one or more surgical instruments to enable the instrument to be driven into the targeted joint. The driver 370 may be passed over a dilator 116, a joint finder 350, a hybrid washer tower 500, or other surgical instrument such that the instrument can be driven into the targeted joint when the head 371 is struck by a mallet or other appropriate device. The canal 373 may have a sufficient diameter and shape to receive and retain one or more surgical instruments.

As shown in FIG. 33, some embodiments may include a washer driver 330, designed to facilitate the insertion and penetration of the hybrid washer tower 500 into the targeted joint. In some examples, the washer driver 330 may have an external diameter that may fit within the inner diameter of the hollow barrel 510 of the hybrid washer tower 500. In such examples, the washer driver 330 may also include one or more coupling pegs 331a and 331b which may be interlocked with the one or more interlock channels 516 according to the methods discussed herein. The coupling pegs 331a, 331b may be slotted into the interlock channels 516 to directly couple the hybrid washer tower 500 and the washer driver 330 together. In other embodiments, the washer driver 330 and the hybrid washer tower 500 may couple without interlocking components. For example, the washer driver 330 and the hybrid washer tower 500 may have complementary, nesting features that allow for a tight pressure fit between the proximal end 502 of the hybrid washer tower 500 and the washer driver 330. The washer driver 330 may have blunt or flat end 332 that can be impacted with a driving tool such as a mallet. The washer driver 330 may be operable to couple to the proximal end 502 of the hybrid washer tower 500 to enable a medical professional to strike the flat end 332 to drive the lateral tangs 512 of the hybrid washer tower 500 into the targeted joint.

Once the distal end 504 of the hybrid washer tower 500 has been positioned proximate to the implant site and driven to its final position, the washer driver 330 may be decoupled from the proximal end 502 of the hybrid washer tower 500. The hollow barrel 510 is then exposed to allow tools, instruments, and implants to pass through to the implant site. In embodiments, a medical professional may then use a drill 260 to create a void for the insertion of the fusion implant 400.

FIGS. 30a-30c show an example drill 260 for bone preparation for fusion implant 400 insertion. The drill 260 may have a recessed portion 261 along its proximal shaft that allows the medical personnel to monitor the depth to which the drill 260 penetrates the targeted joint. The shoulder 262 may be used as a marker used in conjunction with portions, markings, holes, tabs, apertures, or other features on the hybrid washer tower 500 to allow a medical professional to monitor the depth of the drill 260. In embodiments, the shoulder 262 may have a pre-determined marking thereon that when aligned with the opening on the proximal end 502 of the hybrid washer tower 500 indicates that the drill 260 has been advanced to desired depth in the targeted joint to provide a sufficient void for insertion of a joint fusion implant 400.

As shown in FIGS. 30a-30b, the drill 260 may have a threaded or stepped blade portion 263 with numerous concentric blade steps in order to provide a hole with the desired size void cut in the targeted anatomy. The stepped blade 263 with the widest diameter may have a diameter that is less than or equal to the interior diameter of the hollow barrel 510 of the hybrid washer tower 500 to allow the drill 260 to pass through the hybrid washer tower 500 and create a void in the targeted joint. The drill 260 may also be designed to be attached to power instruments, a hand drill or a handle. In embodiments, the drill 260 may have a Jacob's chuck connection 265 so that it may be attached to a powered drill for quick preparation.

Once a void has been created, the drill 260 may be removed from the hollow barrel 510. In embodiments, the joint fusion implant 400 may then be coupled to a distal end 341 of an inserter 340 and the inserter 340 may be progressed through the hollow barrel 510 of the hybrid washer tower 500 to the insertion site.

As shown in FIGS. 35a-35c and FIG. 42, the hybrid washer tower 500 may be configured to accept an inserter 340. The inserter 340 may include a bit recess 344 to couple the head 402 of a fusion implant 400 with the distal end 341 of the inserter 340. The inserter 340 may have a mechanism to allow for variable engagement (e.g., tightening or loosening) of the bit recess 344 with the head 402 of the joint fusion implant 400. In embodiments, the inserter 340 may include an actuator operable by the medical personnel to control a spring-operated element positioned in the bit recess 344 to apply or remove pressure to the head 402 of the joint fusion implant 400 to maintain the joint head 402 in the bit recess 344. The inserter 340 may have a shaft 348 with a corresponding diameter to the hollow barrel 510 of the hybrid washer tower 500 to permit the inserter 340 to pass through the hollow barrel 510 with minimal clearance between the shaft 348 and the outer wall 515 so as to guide the inserter 340 to the implant site without losing alignment. The shaft 348 of the inserter 340 may further include a connection pin 518. The connection pin 518 may be a shaft, arm, pin, bead, column, nub, hemisphere, or other radial protrusion positioned on the shaft 348 of the inserter 340 that may correspond to the shape and size of the interlock channel 516.

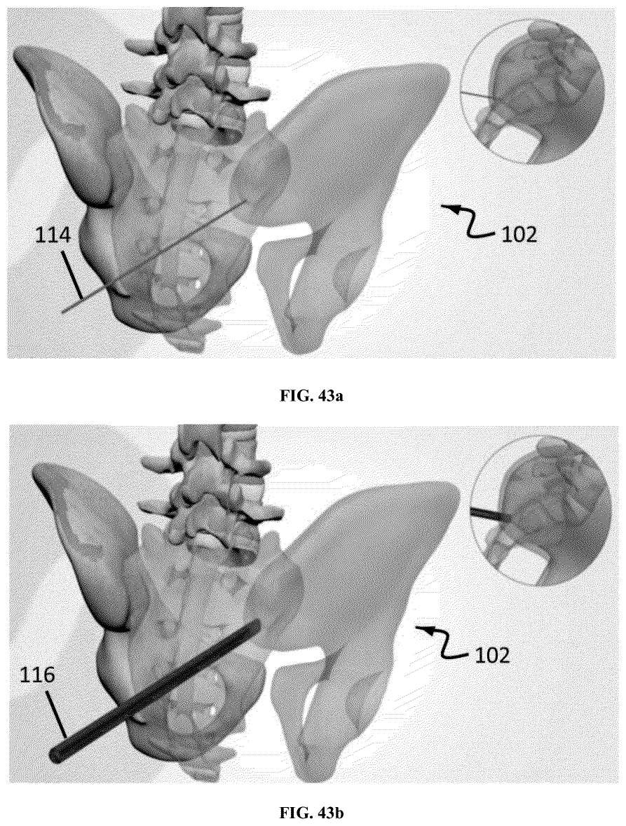

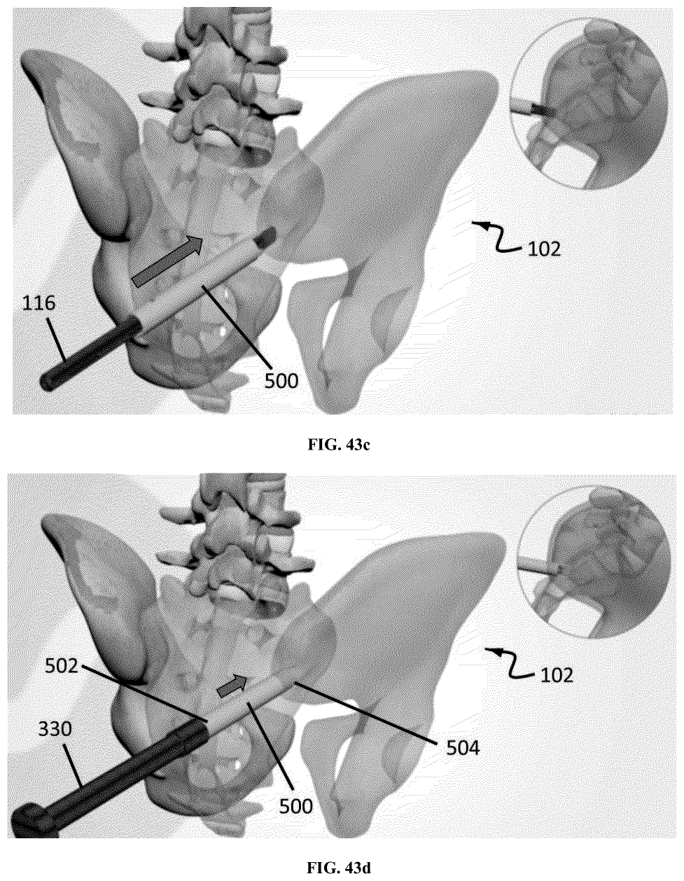

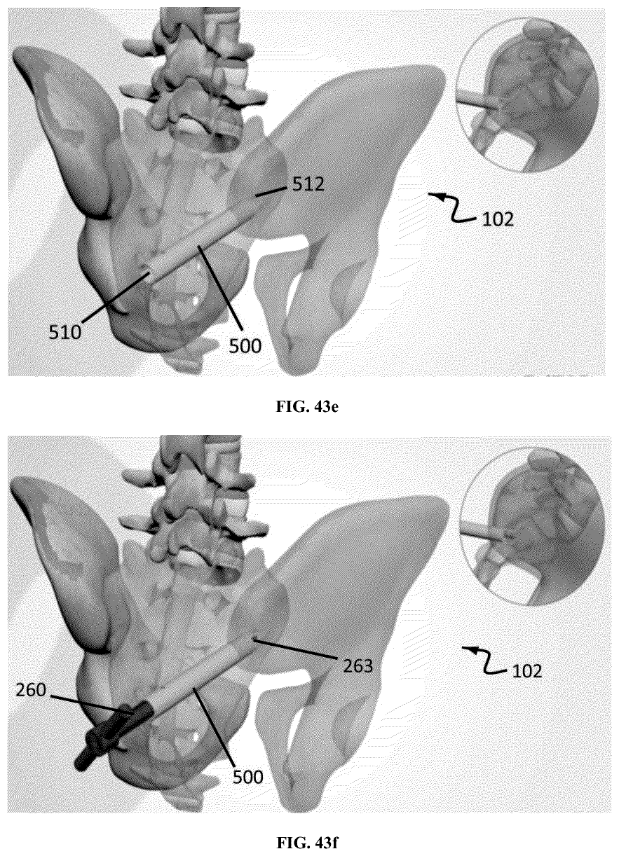

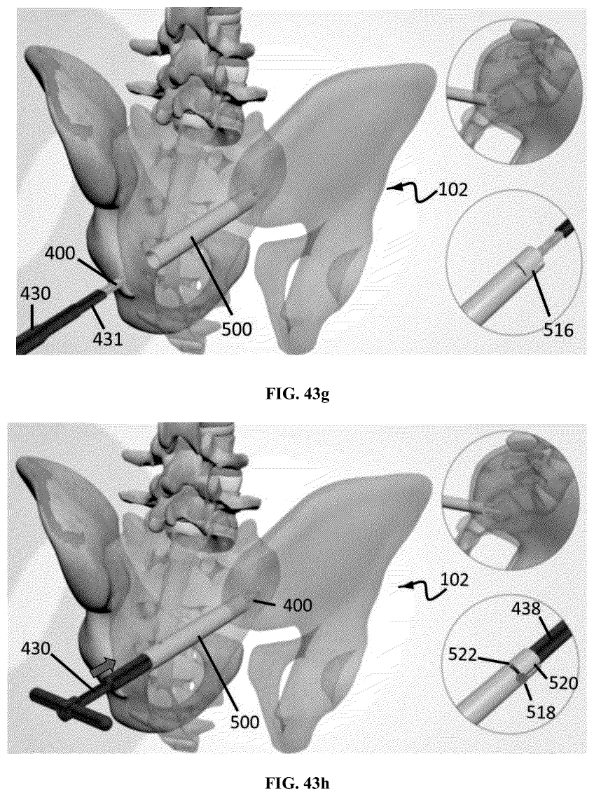

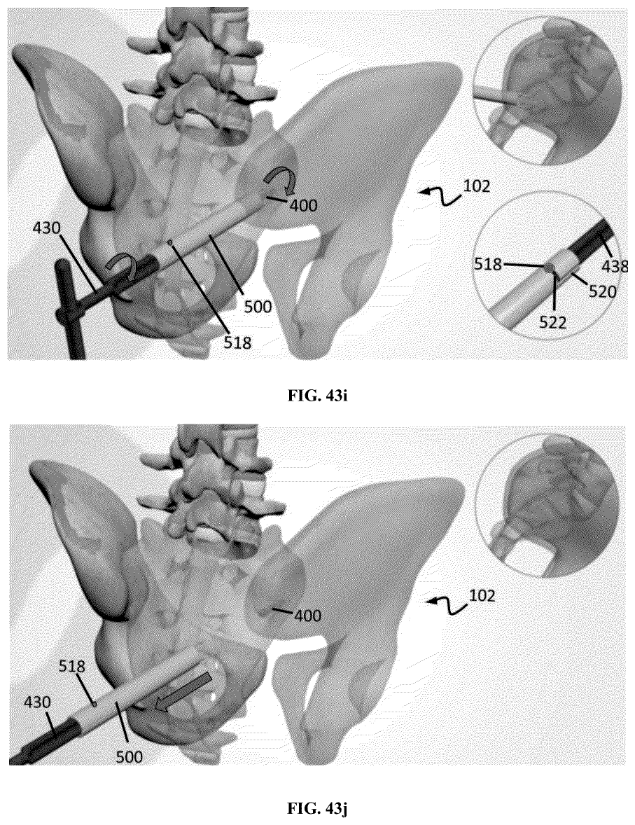

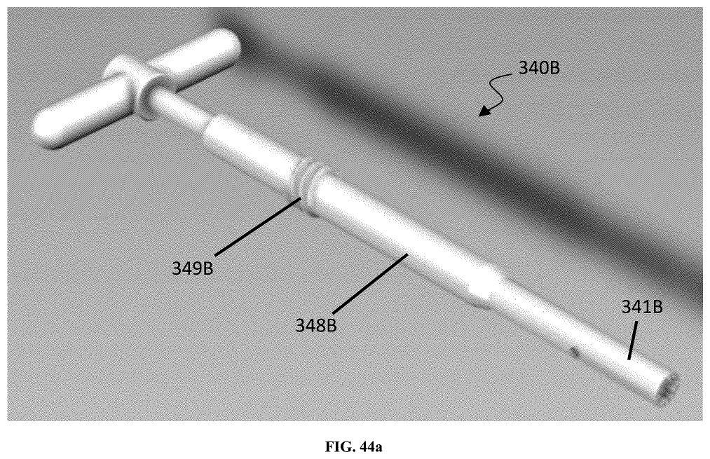

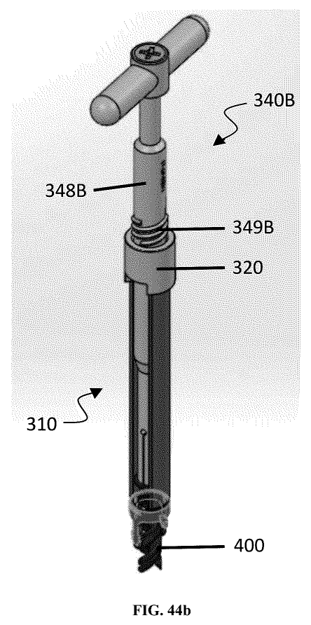

The inserter 340 may be used to advance the joint fusion implant 400 to the targeted joint. As depicted in FIG. 43h, the inserter 340 may be progressed longitudinally through the hollow barrel 510 until it has reached a predetermined terminal, longitudinal position. The terminal, longitudinal position is determined by the length and positon of a longitudinal portion 520 of the interlock channel 516 of the hybrid washer tower 500. The size and position of the interlock channel 516 may be predetermined by medical personnel to ensure that a distal cutting edge 405 of the joint fusion implant 400 is in contact with the SI joint 102 at the implant site when the inserter 340 reaches the terminal, longitudinal position. Movement along the longitudinal portion 520 of the interlock channel 516 may be restricted (i.e., the terminal, longitudinal position) when one or more connection pins 518 positioned on the shaft 348 of the inserter 340 reaches a junction of the longitudinal portion 520 and a transverse portion 522 of the interlock channel 516, restricting any further longitudinal progression of the inserter 340. The medical personnel may then rotate the inserter 340 to cause the at least one lateral fixation element of the joint fusion implant 400 to penetrate the bone tissue of the targeted joint. Additionally, the rotation of the inserter 340 causes the position of the connection pin 518 to progress in the transverse direction through the transverse portion 522 of the interlock channel 516, interlocking the inserter 340 and the hybrid washer tower 500. In embodiments, medical personnel may rotate the inserter 340 (for example, ninety degrees) to affix the joint fusion implant 400 at the implant site and to interlock the inserter 340 with the hybrid washer tower 500 simultaneously.

As depicted in FIGS. 431-44j, the interlocked connection pin 518 and interlock channel 516 may allow the inserter 340 and the hybrid washer tower 500 to move in unison when acted upon by a longitudinal force. Following insertion of the joint fusion implant 400, the joint fusion implant 400 may be decoupled from the distal end 341 of the inserter 340 and a longitudinal pulling force may be applied to retract the inserter 340 from the incision, simultaneously removing the inserter 340 and hybrid washer tower 500 without leaving any break-away components.

In other embodiments, as depicted in FIGS. 44a-44b, the inserter 340B may have a shaft 348B and a distal end 341B configured to attach to the head 402 of the joint fusion implant 400. The inserter 340B may include threads 349B or other fixation elements positioned at the proximal end of the shaft 340B. The threads 349B may be configured to enable the shaft 348B of the inserter 340B to couple with a threaded nut 320 or other threaded portion of the hybrid washer tower 500. As the inserter 340B is rotated to affix the implant 400 at the implant site, the threads 349B may with a threaded component positioned at the proximal end of the hybrid washer tower 500. Accordingly, the interlocked inserter 340B and hybrid washer tower 500 may be removed simultaneously from the incision by medical personnel. The interaction between the threaded nut 320 and the inserter 340B may have additional benefits beyond merely coupling the threaded nut 320 and the inserter 340B. In embodiments, the threaded nut 320 may provide additional mechanical stability to the hybrid washer tower 500 at its proximal end. Furthermore, the threads 349B and the threaded nut 320 may be positioned at a predetermined location relative to the implant site to serve as a depth/insertion control guide, preventing over insertion of the inserter 340B into the SI joint 102. In embodiments, the threads 349B may be either female or male threads and the threaded nut 320 may have opposite, corresponding threading to couple with the threads 349B.

The surgical kits of the present disclosure may also include one or more of the joint fusion implants 400 disclosed herein, and a particular kit may include an exposure device having an internal cross-section that corresponds to a shape of the one or more joint fusion implants 400 that are included in the kit. It is to be appreciated that additional surgical tools or implements may be used with the present disclosure, and that the disclosure is not limited to use of the implements described in this section.

### Fusion Implants

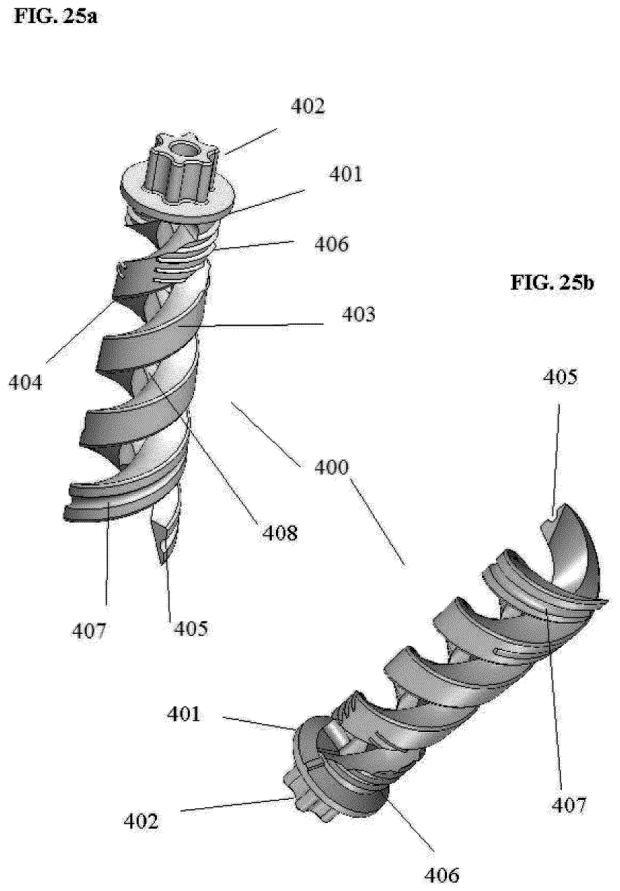

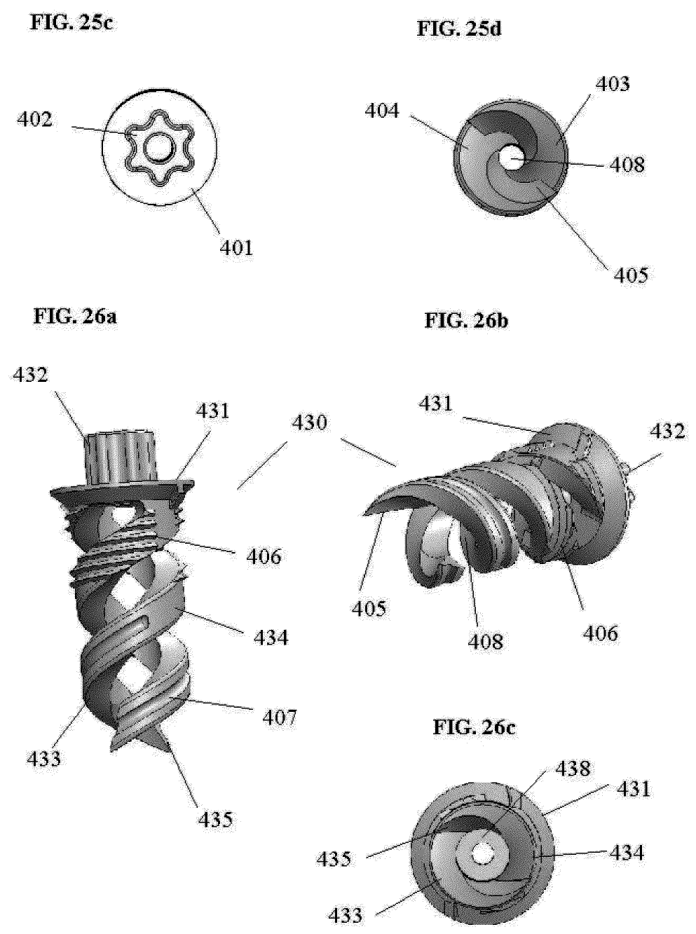

Referring to FIGS. 25a-25c, the present disclosure also relates to fusion implants 400 that include fixation element(s) that mechanically secure the targeted joint (e.g., the sacroiliac joint 102). Additionally, the fusion implants 400 of the present disclosure may compress the targeted joint and/or may deliver bone-growth promoting material into the targeted joint to facilitate the formation of a contiguous piece of bone between the bones of the targeted joint (e.g., from the sacrum 101 to the ilium 100). The joint fusion implants 400 of the present disclosure may be inserted and implanted into the targeted joint. The novel posterior exposure device kit and the combined fixation and stability, and fusion functionalities of the joint fusion implants 400 of the present disclosure may allow for posterior approach that may eliminate the need for patient repositioning or further incisions, resulting in less surgery time, less morbidity, and improved recovery time for the patient. Thus, the surgical methods and fusion implants 400 of the present disclosure allow for a minimally invasive methodology.

Referring to FIGS. 25a-25d and 26a-26c, to create fixation and stability from a posterior implant 400 in the targeted joint, the joint fusion implants 400 of the present disclosure connect and hold the articular surfaces of two adjacent bones of a targeted joint (e.g., the sacrum 101 and ilium 100) together. The fusion implants 400 of the present disclosure may be used to draw the articular surfaces of the targeted joint together. Additionally, the fusion implants 400 of the present disclosure may include a gap, space, or cavity (i.e., a central canal 408) for holding and delivery bone growth stimulating material to stimulate fusion of the articular surfaces, such as autologous bone, allograft, BMP, etc. In some embodiments, a combination of joint compression and bone-growth materials may be combined to provide for stable and robust bone fusion. The joint fusion implant 400 may create stress and pressure on the bone tissue by mechanically drawing the bones together, thereby bridging the two articulating bones for a fusion site. This mechanism promotes bone remodeling (e.g., as according to Wolff's Law) and the bone-growth material may further aid in the bone fusion process.

Referring to FIGS. 25a-25d, 26a-26c, and 27a-27f, in some embodiments of the fusion implant 400, and without limitation, the joint fusion implant 400 may have one or more helical anchors 403, 404 for insertion into bone tissue. For example, and without limitation, a joint fusion implant 400 may include a single helix 473 or multiple helices 453, 454 (e.g., 2 or 3 helices) that may be concentric and/or interwoven. In some embodiments, and without limitation, the helical path of the helical fixation anchors 403, 404 may have a uniform diameter from their body 401 to the distal cutting edge 405 thereof. However, in other embodiments, and without limitation, the helical path of the helical fixation anchors 403, 404 may taper outward from body 401 to the distal cutting edge 405 of the helical path to create a conical shape to the helical path. After being inserted into the targeted joint the fusion implant 400 may be rotated in a range of about 90° to about 360° (e.g., about 180° to about 270°, or any value or range of values therein) such that helical anchors 403, 404 may engage the articular surfaces of adjacent bones in the targeted joint (e.g., the ilium 100 and the sacrum 101) so that the fusion implant 400 can hold the bones together and stabilize the targeted joint.

Without limiting the present disclosure, FIGS. 25a-25d show an example fusion implant 400 having two helical anchors 403, 404. The implant 400 has a body 401 (i.e., a proximal end) attached to two helical anchors 403,404, which may be concentric and interwoven. Each of the helical anchors 403, 404 may have a distal cutting edge 405 (i.e., a distal end) that may be operable to penetrate bone tissue in joint targeted for fusion. The distal cutting edges 405 of each of the helical anchors 403 and 404 may be on opposite sides of the fusion implant 400 such that as the implant 400 is advanced into the targeted joint, the helical anchor 403 engages a first articulating bone (e.g., the ilium 100) and helical anchor 404 engages a second articulating bone (e.g., the sacrum 101). The body 401 of the fusion implant 400 may have a head 402 having a structure that is complementary to a bit recess 344 in the inserter 340. In other embodiments, the head 402 of the joint fusion implant 400 may include notches or slots 452, 472 that may be engaged by an inserter 340 having grasping mechanisms complementary thereto. The implant 400 may have an outer diameter in a range of about 8 mm to about 20 mm (e.g., about 12 mm to about 18 mm, or any other value or range of values therein). The joint fusion implant 400 may have a central canal 408 operable to hold bone growth-promoting materials that provides for mechanical stability and promotes the formation of a contiguous piece of bone between two bones in the targeted joint (e.g., the sacrum 101 and the ilium 100).

Each of the anchors 403 and 404 may have outwardly facing secondary fixation elements to improve the bite and purchase of the joint fusion implant 400 when it is implanted into the joint. For example, the anchors 403 and 404 may having external threading 406 on at least a portion of the outer surface of the anchors 403 and 404. The external threading 406 provides further fixation structures that improve the purchase and reliability of the position of the fusion in the bone tissue.

The helical anchors 403 and 404 may also include further secondary fixation elements to assisting in the engagement of the anchors 403 and 404 with the bone. For example, the anchors 403 and 404 may further include outwardly facing fluting 407 along the length of at least a portion of the outer surface of the anchors 403 and 404. The fluting 407 may both (1) allow bone tissue displaced by the implantation of the joint fusion implant 400 to be passed along the fluting 407 provide a pathway for expulsion from the surgically formed void in the targeted joint, and (2) improve the bite and purchase of the joint fusion implant 400 when it is implanted into the targeted joint.

The anchors 403 and 404 may also have a distally increasing radius. Each of the anchors 403, 404 may follow a concentric, but expanding spiral pattern from their respective proximal ends attached to the body 401 to their respective distal ends that terminate at distal cutting edges 405, with each turn of the spiral pattern having a larger radius than the preceding turn. As a result, the diameter of the interwoven anchors 403 and 404 increases incrementally from the body 401 to the distal cutting edges 405. The retrograde tapering of the spiral pattern of the anchors 403, 404 provides a mechanism for drawing together the articulating surfaces of the articulating bones of the targeted joint as the joint fusion implant 400 is driven into the targeted joint. As the inserter 340 rotates the joint fusion implant 400 into the targeted joint, the anchors 403, 404 dig into the prepared bone tissue, drawing the articulating surfaces together due to the retrograde angling of the anchors 403, 404.

In other embodiments, and without limitation, a columnar fusion implant 430 may have anchors 433, 434 having spiral pattern has a consistent diameter throughout the length of the columnar fusion implant 430. FIGS. 26a-26c show an example columnar fusion implant 430 having two helical anchors 433, 434. The implant 430 has a body 431 attached to two helical anchors 433, 434, which may be concentric and interwoven. Like the embodiment shown in FIGS. 25a-25d, each of the helical anchors 433, 434 may have a distal cutting edge 435 that may be operable to penetrate bone tissue in joint targeted for fusion. The distal cutting edges 435 of each of the helical anchors 433, 434 may be on opposite sides of the fusion implant 430 such that as the implant 430 is advanced into the targeted joint, the first helical anchor 433 engages a first articulating bone (e.g., the ilium 100) and the second helical anchor 434 engages a second articulating bone (e.g., the sacrum 101). The body 431 of the fusion implant 430 may have a head 432 having a structure that is complementary to a bit recess 344 in the inserter 340. In other embodiments, the head 432 of the columnar fusion implant 430 may include notches or slots 452, 472 that may be engaged by an inserter 340 having grasping mechanisms complementary thereto. The implant 430 may have an outer diameter in a range of about 8 mm to about 20 mm (e.g., about 12 mm to about 18 mm, or any other value or range of values therein). The columnar fusion implant 430 may have a central canal 438 operable to hold bone growth-promoting materials that provides for mechanical stability and promotes the formation of a contiguous piece of bone between two bones in the targeted joint (e.g., the sacrum 101 and the ilium 100).

Each of the anchors 433 and 434 may have outwardly facing secondary fixation elements. For example, the anchors 433 and 434 may include threading on at least a portion of the outer surface of the anchors 433 and 434 to improve the bite and purchase of the joint fusion implant 430 when it is implanted into the joint. The helical anchors 433 and 434 may also include further secondary fixation elements. For example, the helical anchors 433 and 434 may include outwardly facing fluting 407 along the length of at least a portion of the outer surface of the anchors 403 and 404.

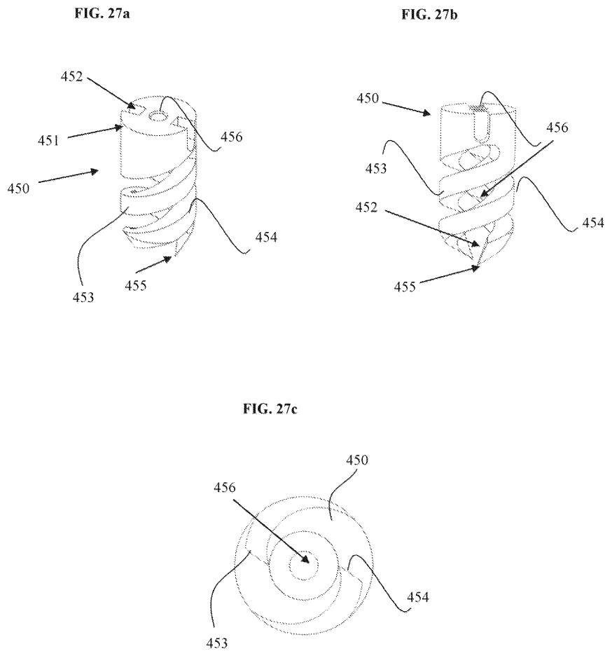

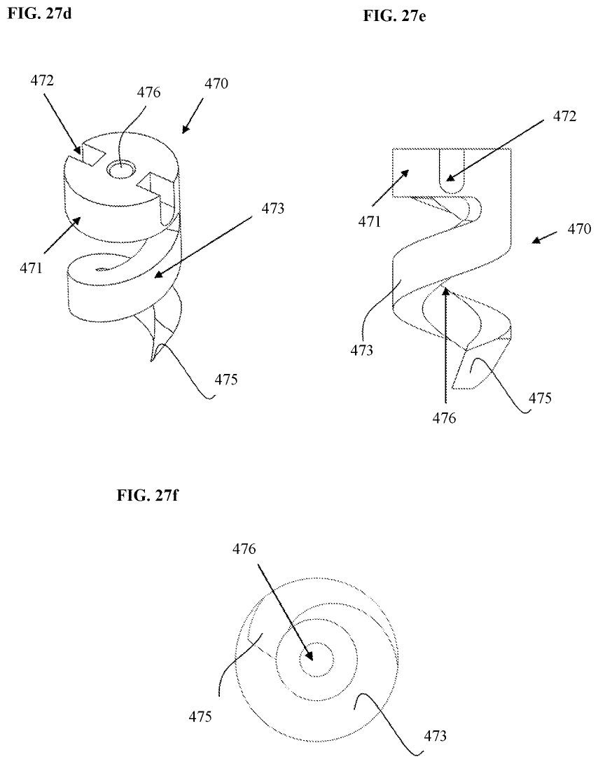

In other embodiments, and without limitation, the joint fusion implant 400 may have one or more helical anchors 403 that have a smooth exterior, without threading or fluting 407, as show in FIGS. 27a-27c. For example, and without limitation, a polygonal fusion implant 450 may include two helical anchors 453 and 453 having a polygonal cross-sections (may be round or elliptical in other embodiments) that concentrically spiral around one another. The fusion implant 450 has a body 451 attached to the helical anchors 453 and 454 at their proximal ends. The helical anchors 453 and 454 may each have a distal cutting edge 455 that may be operable to penetrate bone tissue in joint targeted for fusion. The body 451 of the fusion implant 450 may have notches or slots 452 in a perimeter thereof that may be engaged by the inserter 340 or driver 370 as described herein (e.g., by an inserter 340 having fork arms). Alternatively, the joint fusion implant 450 may have a body 451 having a different engagement structure, such as a star-shaped driver pattern.

In still other embodiments, and without limitation, the fusion implant 470 may have a single helix 473 attached to the body 471 of the fusion implant 470. For example, and without limitation, FIGS. 27d-27f illustrate an example joint fusion implant 470 that may include a single helical anchor 473. The fusion implant 470 has a body 471 attached to the helical anchor 473, which may have a distal cutting edge 475 that may be operable to penetrate bone tissue in joint targeted for fusion. The body 471 of the fusion implant 470 may have notches or slots 472 in a perimeter thereof that may be engaged by the inserter 340 or driver 370 as described herein. Alternatively, the joint fusion implant 470 may have a body 471 having a different engagement structure, such as a star-shaped driver pattern.

### Surgical Methods

In some embodiments, the methods of the present disclosure can substantially fuse the targeted joint (e.g., the SI joint 102), such that movement in the joint is minimized or substantially eliminated, thereby diminishing or substantially eliminating the patient's pain and discomfort. More specifically, an improved, combined approach for both mechanical holding and surgical fusion using the novel surgical tools and tool sets of the present disclosure, as described above. Specifically, with respect to some embodiments, an approach is described to address the targeted joint (e.g., an SI joint 102) through a posterior approach. In some embodiments, and without limitation, the surgical fusion of the sacrum 101 and an ilium 100 may be accomplished with a single posteriorly inserted fusion implant device 400 alone. In some implementations, a fusion implant 400 may be delivered to the SI joint 102, placed between the sacrum 101 and ilium 100 and positioned about between a posterior inferior iliac spine and a superior lateral aspect of the sacrum 101 of the SI joint 102. In other implementations, a fusion implant 400 may be delivered into the sacroiliac joint 102 at a position below a posterior superior iliac spine (PSIS) into the SI joint 102 space between the ilium 100 and sacrum 101 of the sacroiliac joint 102. For example, a fusion implant 400 may be delivered into the sacroiliac joint 102 at a position in close proximity to and just inferior to a posterior superior iliac spine between the ilium 100 and sacrum 101. The incision may be made just inferior to the SI joint 102 and the instrumentation may be positioned such that the fusion implant 400 is established in the SI Joint 102 in close proximity to the posterior superior iliac spine.

In some implementations, and without limitation, surgical fusion may be accomplished with the delivery of a plurality of fusion implants 400 into the SI joint 102. A fusion implant 400 may be delivered to the SI joint 102 space positioned about between a posterior inferior iliac spine and a superior lateral aspect of the sacrum 101 of the sacroiliac joint 102. In some implementations, an additional fusion implant 400 may be delivered into the sacroiliac joint 102 at a position about between a posterior superior iliac spine and a superior lateral aspect of the sacrum 101 of the sacroiliac joint 102.

### Break-Away Methods

In some embodiments, and without limitation, the method may involve the posterior insertion of a fusion implant 400, including the steps of creating an incision proximal to the patient's SI joint 102 (e.g., below a posterior superior iliac spine between the ilium 100 and sacrum 101 of the SI joint 102), introducing a guide wire 114, dilating the incision, inserting a cannula 250 into the incision over the dilator 116 or joint finder 350 and guide wire 114, creating a void in the SI joint 102, inserting a washer 310, and inserting a fusion implant 400 into the void through the washer 310 such that it engages with the articular surfaces of the sacrum 101 and ilium 100. Optionally, and as described in more detail below, a sensing device/technique can be used to locate the proper/suitable joint detection/placement in lieu of an initial k-wire for detection. Similarly, radio-opaque dye can be injected (e.g., via a syringe 259 into a sensing cannula 250) to effect joint confirmation, e.g., secondary joint confirmation. In this manner, once the joint location/placement is confirmed via the sensory mechanisms, a standard k-wire can be employed to commence the procedure as described herein.

In some embodiments, without limitation, include some or all of the following steps, preparing the patient for surgery (e.g., positioning the patient in a prone position to provide the surgeon access to the SI joint 102, general or local anesthesia, and the like), locating the SI joint 102 and an incision point for access to the SI joint 102 (e.g., by blunt finger palpation), insertion of a guide pin 114 or guide wire 114 to create an incision, insertion of a dilator 116 over the guide pin 114 and impacting the dilator 116 to dilate the incision to a width through which instruments may be passed, inserting a cannula 250 over the dilator 116, securing the cannula 250 in position (e.g., with fixing pins 126), removing the dilator 116, inserting a drill 260 through the channel 256 of the cannula 250, using the drill 260 in the channel 256 of the cannula 250 to displace bone and other tissue at the SI joint 102 thereby creating a void, removing the drill 260, pairing a washer 310 with a washer driver 330, inserting the washer 310 into the sacroiliac joint 102 utilizing the washer driver 330, removing the washer driver 330 from the washer 310 once the washer 310 is positioned at the sacroiliac joint 102, loading a fusion implant 400 onto an inserter 340 and inserting the fusion implant 400 and inserter 340 through the cannula 250 and through a ring 311 of the washer 310 until the implant 400 is positioned proximate to the void in the patient's SI joint 102, driving the fusion implant 400 into the joint to engage the articular surfaces of the SI joint 102 (e.g., by rotating and driving the fusion implant 400 forward into the joint), breaking away guidance tabs 315a, 315b on the washer 310, removing all instruments, and closing the incision.

It is to be appreciated that the washer 310 can resist rotational forces while the helical (i.e., screw) implant 400 resists flexion/extension. Together they resist lateral and medial bending. Overall, the system of this embodiment is an effort to lock the ilium 100 and sacrum 101 in all six (6) degrees of freedom. Additionally, the washer 310 can keep the fusion implant 400 from stripping out as there is included a metal-on-metal surface contact/abutment that keeps the fusion implant 400 (i.e., helical implant 400) from being over-tightened.

While the above description employs a drill 260 to create a void, some aspects do not utilize a drill 260 to create this void. Rather, it is to be appreciated that a drill 260 of the same or similar circumference as the washer 310 can be utilized to create a countersink in the ilium 100 and sacrum 101 to seat the washer 310. In other words, the countersink is used such that the washer 310 does not sit proud to the bone and irritate soft tissue. Moreover, use of the drill 260 also provides decorticatation to allow the fusion implant 400 (i.e., helical implant 400) to start threading into place easier. It is to be appreciated that, in this technique, the drill 260, if used, is merely used to create the countersink and that the implant 400 itself creates the void as it is applied or screwed into the joint. In aspects, bone material can be placed into the helical implant 400 prior to implantation or post-implantation, e.g., via the back cannulation of the implant 456, 476.

In some implementations, without limitation, include some or all of the following steps, preparing the patient for surgery (e.g., positioning the patient in a prone position to provide the surgeon access to the SI joint 102, general or local anesthesia, and the like), locating (and marking) the SI joint 102 and an incision point for access to the SI joint 102 (e.g., by blunt finger palpation), inserting of a guide pin 114 or guide wire 114 to create an incision, inserting of a first cannula 255 over the guide pin 114 or guide wire 114 into the joint space, injecting a radio-opaque dye 259a through the first cannula 255 into the joint space, conducting an imaging process to visualize the dye in the joint to conform placement of the first cannula 255 in the joint space, inserting of a guide wire 114 through the first cannula 255 into the joint space and removing the first cannula 255, insertion of a joint finder 350 over the guide wire 114 and impacting the joint finder 350 to dilate the incision to a width through which instruments may be passed and decorticate the articular surfaces, inserting a cannula 250 over the dilator 116, passing a drill 260 over the joint finder 350, using the drill 260 to prepare the bone tissue in the SI joint 102 providing an entry point in the bone tissue for the fixation elements of a joint fusion implant 400, removing the drill 260, passing a washer 310 over the joint finder 350 and driving the distal end of the washer 310 into the SI joint 102, removing the joint finder 350 from the SI joint 102, loading a fusion implant 400 onto an inserter 340 and inserting the fusion implant 400 and inserter 340 through the cannula 250 and through the washer 310 until the implant 400 is positioned proximate to the entry point in the patient's SI joint 102, driving the fusion implant 400 into the joint to engage the articular surfaces of the SI joint 102 (e.g., by rotating and driving the fusion implant 400 forward into the joint with the inserter 340), breaking away guidance tabs 315a, 315b on the washer 310, removing all instruments, and closing the incision. The radio-opaque dye 259a may include various materials such as iodine, indigo carmine, lipiodol, indocyanine green, and mixtures thereof. The radio-opaque dye 259a position may be imaged using computerized tomography scan (CT), x-ray imaging, or other appropriate imaging methods.

The methods of the present disclosure (and tools of the present disclosure) are now further described with reference to the Figures. Although the methods are described with respect to the use of certain tools, the methods of the present disclosure may be performed in addition to or in conjunction with one or more of the known other tools used in the relevant art.

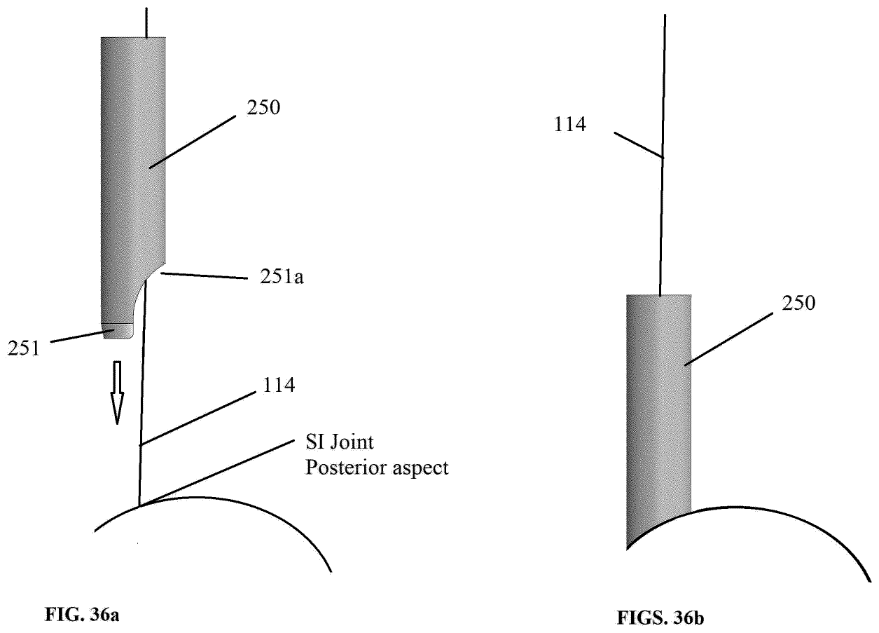

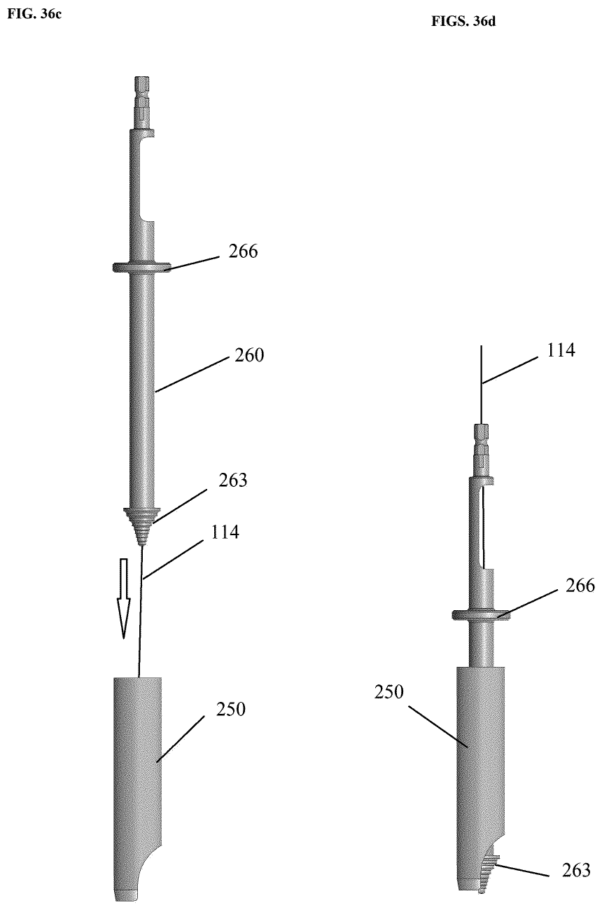

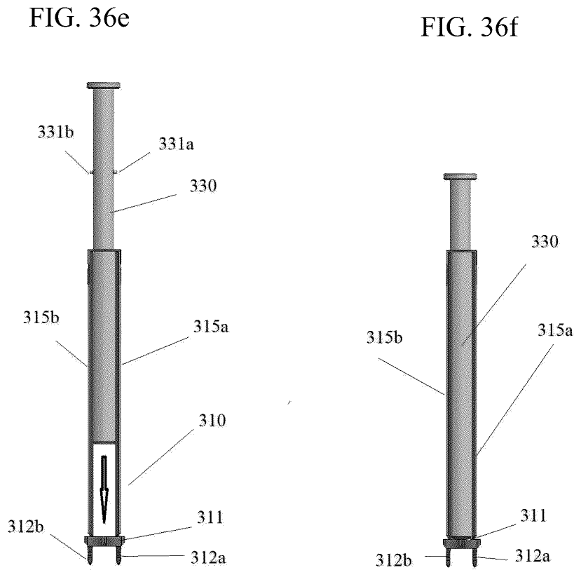

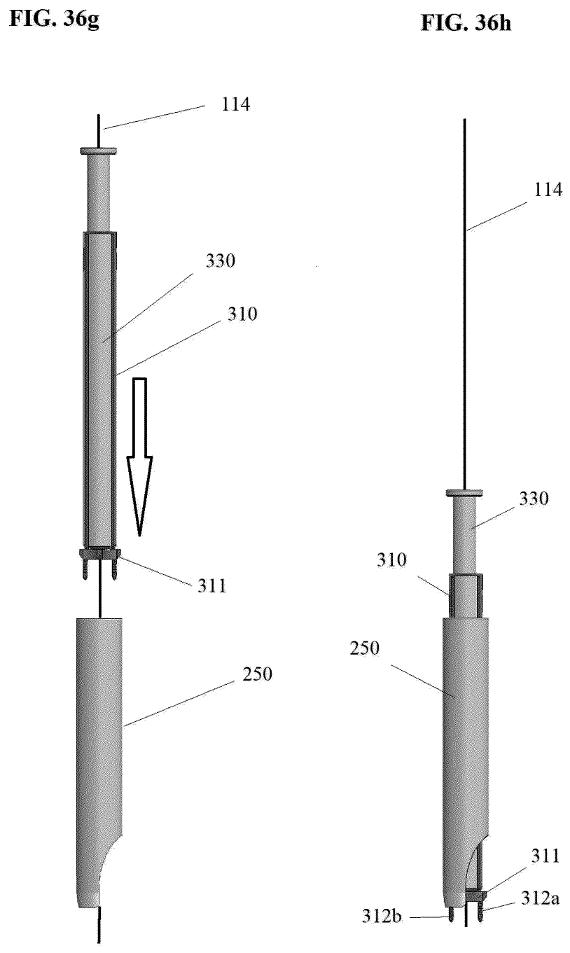

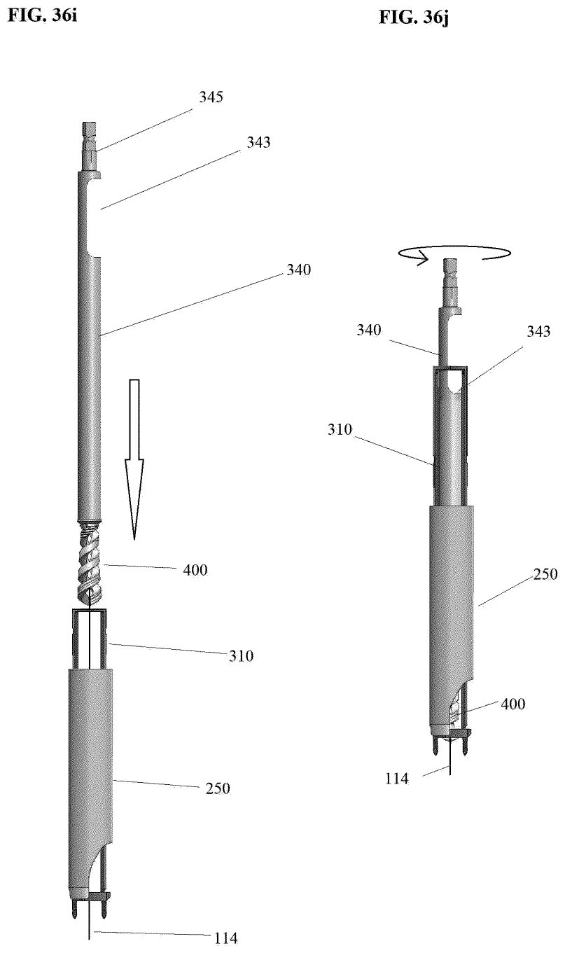

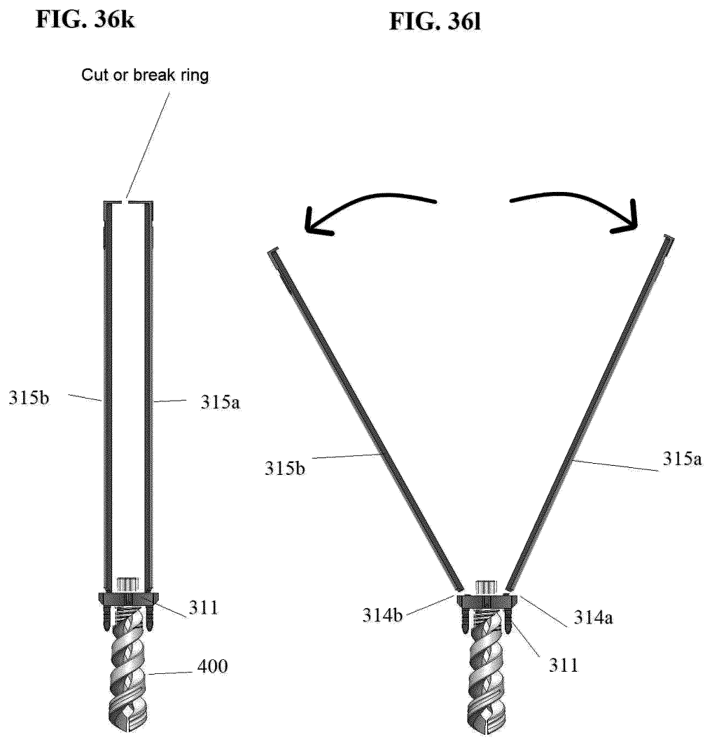

FIGS. 36a-361 illustrate an example surgical procedure for fusing an SI joint 102 with one or more fusion implants 400 inserted in the SI joint 102. The procedure includes positioning a patient in the prone position and administering either a local or general anesthetic. Blunt finger palpation may be used to locate the patient's iliac wing 100 and the SI joint 102. As shown in FIGS. 1-7, the SI joint 102 is located between the iliac wing 100 and the sacrum 101 at the base of the pelvis 201. The SI joint 102 is fully enclosed between the iliac wing 100 and the sacrum 101 and occluded for direct visualization by the iliac wing 100. Additionally, the iliac crest 200, the posterior superior and inferior iliac spines, and the pedicle 104 of vertebrae S1 can be observed in this view. The iliac crest 200 may provide a posterior landmark for the entry point of the exposure device of the present disclosure at the posterior iliac crest 200, and can be palpated to find the general location of the SI joint 102. Alternatively, suitable locations for an incision may be determined by imaging methods (e.g., x-ray), or any other suitable method. The incision may be positioned between a posterior inferior iliac spine and a superior lateral aspect of the sacrum 101 of the SI joint 102. In other implementations, the incision may be positioned below a posterior superior iliac spine between the ilium 100 and sacrum 101 of the sacroiliac joint 102.

FIGS. 8-24 show tools that may be used to establish an incision and assist in establishing a void in the sacroiliac joint 102. A joint probe 112 may be used to identify the insertion area on the posterior side of the SI joint 102. The area of the SI joint 102 may be probed until the rounded geometry of the joint probe 112 finds or drops into the proper position in the SI joint 102, where an incision may be properly made. Subsequently, a guide pin 114 may be inserted through an aperture 113 in the joint probe 112 and into the patient to create an incision in the SI joint 102. Alternatively, the incision may be made by any suitable method, including scalpel or other cutting or dissection tool. The incision may be made proximal to the patient's SI joint 102, allowing the joint to be accessed by the exposure device. The guide pin 114 may be advanced until its distal end 115 is in contact with the SI joint 102 or at least partially within SI joint 102.

A dilator 116 may be used to dilate the incision. As an example, dilator 116 may be slotted over guide pin 114 through a hollow column 213 running the length of the dilator 116. The proximal end of the dilator 116 may be slotted over the guide pin 114, and dilator 116 may then be advanced to or near the SI joint 102 through the incision. As dilator 116 enters the incision, the tapered distal end 118 pushes the patient's tissue aside, thereby dilating the incision to accommodate an exposure device as described herein. A joint cutting assembly that includes the dilator 116 and a T-handle 119 engaged with a proximal end 117 of the dilator 116 may be used to further drive the dilator 116 into the incision to a desired depth to sufficiently expose the SI joint 102. Alternatively, an impactor 136 may be used to further drive the dilator 116 into the incision to a desired depth.

FIG. 36a illustrates the placement of the cannula 250 over the guide wire 114. The cannula 250 can be positioned over the coupled dilator 116 and the guide wire 114 in the incision and the dilator 116 may be removed from the incision. The cannula 250 is advanced toward SI joint 102 through incision until the distal end 251 is in contact with the SI joint 102 or is positioned proximal to the SI joint 102 and in contact with the sacrum 101 and/or ilium 100. The distal end 251 may have a round geometry and/or a tapered rounded profile that is operable to engage the SI joint 102 with minimal damage to soft and connective tissue in and around the posterior side of the SI joint 102. The distal end 251 of the cannula 250 may also have an arched, cut-away portion that accommodates the bony anatomy of the SI joint 102 (e.g., the iliac crest 200). It is to be appreciated that the cannula 250 may have other distal geometries, e.g., where there is no cut-away and the distal end 251 has a complete circular circumference. It should be understood that the cannula 250 may have other cross-sectional shapes as well (e.g., elliptical, polygonal [pentagonal, hexagonal, etc.], Reuleaux shapes, and other applicable shapes), e.g., to accommodate the shape of various fusion implants 400. The cannula 250 may also further dilate the incision.

The cannula 250 may include exterior features (not shown) that allow for the stabilization of the cannula 250 in the SI joint 102. For example, the cannula 250 may include holes, slots or tabs through which fixing pins 126 may be slotted to stabilize the cannula 250. In some embodiments, and without limitation, fixing pins 126 can penetrate the skin and/or flesh and tissue of a human. It is to be appreciated that any suitable method of stabilizing exposure device may be used. The dilator 116 may be removed from the cannula 250 either before or after the fixing pins 126 are inserted. In other embodiments, and without limitation, the cannula 250 may be stabilized by attachment to a surgical or stabilizing arm to hold the exposure device in a static and stable position.

FIGS. 36c-36d illustrate insertion of drill 260 into the incision through the cannula 250. The drill 260 may be connected to a power drill configured for medical procedures. The drill 260 may have cylindrical outer walls that allow the drill 260 to freely spin within the hollow channel 256 of the cannula 250. The cylindrical outer wall may comprise a low-friction material that facilitates smooth spinning of the drill 260 within the channel 256. The distal end of the drill 260 may be inserted into the cannula 250 over guide wire 114 and may be advanced to a predetermined point. In some examples, and without limitation, the distal end of the drill 260 may extend past the distal end 251 of the cannula 250 by a pre-determined amount that is equal to or less than a length of an insertable portion of a fusion implant 400. The drill 260 may have a shoulder 262 that provides indication of the depth to which the drill 260 should be advanced into the SI joint 102.

When the shoulder 262 meets the proximal end of the cannula 250, the drill 260 may be advanced to the desired pre-determined depth. When advanced into the SI joint 102, the drill 260 may create a void in the patient's SI joint 102 by displacing portions of sacrum 101 and ilium 100. In such examples, the drill 260 may be configured such that it will contact the patient's SI joint 102 at a desired portion of the joint and, once activated, will create a void of a desired depth. The void may be configured to receive a fusion implant 400 as described herein or other joint repairing appliance or bone graft for fusing the SI joint 102. Other joint repairing appliances or apparatus may include a polyether ether ketone (PEEK) implant, a titanium implant, etc. As an example, and without limiting the present disclosure, the implant may be a fusion implant like one of those shown in FIGS. 25a-27f.

As shown in FIGS. 8-24, the subject method may utilize several implements that may be inserted through the cannula 250 and into the void in the SI joint 102 to prepare the void for receiving a fusion implant 400. For instance, a box chisel 131 and/or a rasp 132 may be inserted into the void through the cannula 250 to expand and clear tissue from the void to facilitate a clean and efficient insertion of the fusion implant 400 into the void.

As shown in FIGS. 36e-36f, the washer 310 may be paired with a washer driver 330 to prepare the washer 310 for insertion into the SI joint 102. The washer 310 includes a washer ring 311 at its distal end that may include lateral tangs 312a, 312b for penetrating tissues in the SI joint 102 and establishing a stable purchase of the washer 310 before a joint fusion implant 400 is inserted into the SI joint 102. The washer 310 may further include guidance tabs 315a, 315b that connect to the washer ring 311 at break-away connection points 314a, 314b. The guidance tabs 315a, 315b may be operable to be engaged with a washer driver 330 in order to couple the washer 310 and the washer driver 330 together. Each guidance tab 315a, 315b may include coupling channels 316a, 316b (see, e.g., FIGS. 32a and 32c) that may be engaged with coupling pegs 331a, 331b of the washer driver 330. As shown in FIG. 36e-36f, the washer driver 330 may be nested within the hollow of the washer 310, defined by the guidance tabs 315a, 315b, and the coupling pegs 331a, 331b may be slotted into the coupling channels 316a, 316b to secure the washer 310 and washer driver 330 together for insertion of the washer 310 into the SI joint 102 through the cannula 250.

FIGS. 36g-36h show the process of insertion of the coupled washer 310 and washer driver 330 being inserted into the cannula 250 over the guide wire 114 and then driven through the cannula 250 (e.g., by mallet or other driving tool) such that the washer 310 is inserted into the SI joint 102. The lateral tangs 312a, 312b are operable to penetrate tissue in the targeted joint by being driven into the joint, e.g., by impaction. The lateral tangs 312a, 312b may include biting or gripping features (e.g., serrations, barbs, ridges etc.) that provide for structures that facilitate firm lodging in the tissue (e.g., connective tissues, cartilage, prepared bone surface, etc.). Such biting or gripping features may have a retrograde angle directed toward the proximal end of the washer 310 to prevent dislodgement of the washer 311 from the targeted joint. The lateral tangs 312a, 312b may be arranged such that they are diametrically or substantially diametrically positioned on and extend from a first surface of the washer ring 311 such that they can be driven between two adjacent articular surfaces of bones in the target joint, allowing penetration of the joint by both of the tangs 312a, 312b. The washer 310 establishes a stable purchase in the SI joint 102. The washer ring 311 may have a second surface on its proximal side to operatively engage with the body 401 of the fusion implant 400, such that the fusion implant 400 and the washer ring 311 may couple and remain at the implant site following the procedure. The distal surface or body 401 of the fusion implant 400 may have a complementary surface to the second surface of the washer ring 311, such that the body 401 of the fusion implant 400 nests snugly and evenly in the washer ring structure 311.

FIGS. 36l-36j illustrate the use of an inserter 340 to insert a fusion implant 400 into the void in the SI joint 102. The inserter 340 may be inserted into the cannula 250 once the washer driver 330 has been decoupled from the guidance tabs 315a, 315b of the washer 310 and removed from the cannula 250. The inserter 340 is operable to engage with a joint fusion implant 400 and insert the joint fusion implant 400 into the targeted joint. The inserter 340 may be engaged with the head 402 of a joint fusion implant 400 and then used to advance the joint fusion implant 400 through the cannula 250 and into the targeted joint. The inserter 340 may have a distal end 341 configured to attach to the joint fusion implant 400, e.g., having a bit-receiving recess 344 (e.g., star, hex, or other functional shape) for receiving a complementary head 402 of a joint fusion implant 400. The inserter 340 may have a mechanism for tightening the engagement of the bit recess 344 with the head 402 of the joint fusion implant 400. For example, an actuator operable by the medical personnel may control a spring-operated element within the bit recess 344 that reversibly applies pressure to the head 402 of the joint fusion implant 400 to maintain the joint head 402 in the bit recess 344.

The inserter 340 may have a recessed portion 342 along its proximal shaft having a cutaway 343 that allows the medical personnel to monitor the depth to which the inserter 340 penetrates the targeted joint (e.g., the distal end of the cutaway 343 can be used as a marker). The cutaway 343 may act as a marker to be used in conjunction with the markers on the guidance tabs 315a, 315b to determine when the inserter 340 has reached the desired depth to deliver a joint fusion implant 400 into the targeted joint. For example, at least one guidance tab 315a, 315b may be marked to indicate the point to which the distal end of the cutaway 343 of the inserter 340 should be advanced to completely insert the fusion implant 400 into the targeted joint. In other examples, at least one guidance tab 315a, 315b may include two marks, a proximal mark to indicate the point to which the distal end of the cutaway 343 should be advanced to place the distal cutting edge 405 of the fusion implant 400 at superficial aspect of the bones in the joint, and a distal mark to indicate the point to which the cutaway 343 of the inserter 340 should be advanced to completely insert the fusion implant 400 into the targeted joint. A first proximal position marker on a guidance tab 315a of the washer 310 may indicate to the medical personnel that the joint fusion implant 400 is at the superficial aspect of the targeted joint when the cutaway 343 of the inserter 340 is aligned therewith.

Once the inserter 340 has been advanced to the predetermined position at which the distal cutting edge 405 of the joint fusion implant 400 is at the superficial aspect of the SI joint 102, linear advancement of the inserter 340 is arrested, and the fusion implant 400 is positioned to be rotated while it is advanced, such that the fixation elements of the fusion implant 400 engage and penetrate the articular surfaces of the bone to fix the articular surfaces relative to one another. The medical personnel may then rotate the inserter 340 using a driving tool to cause the fixation element(s) of the joint fusion implant to penetrate the bone tissue of the targeted joint. The inserter 340 may have a head 432 comprising a Jacob's chuck connection so that it may be attached to a driving tool. The medical personnel may continue rotating the inserter 340 until the distal end of the cutaway 343 (or other marker) is aligned with a second distal position marker on a guidance tab 315a of the washer 310, which indicates that the joint fusion implant 400 is fully implanted into the bone tissue. When the fusion implant 400 is fully advanced, the distal portion of the body 401 of the fusion implant 400 may be nested in the complementary surface of the ring 311 of the washer 310. This provides a stable, even and snug engagement and position between the washer 310 and the fusion implant 400.

Once the fusion implant has been fulling inserted into the SI joint 102, the inserter 340 may be disengaged from the fusion implant 400 and removed from the cannula 250, and the cannula 250 and guide wire 114 can be removed from the SI joint 102, leaving the washer 310 exposed in the incision. FIGS. 36k and 34l show the process of removing the guidance tabs 315a, 315b from the washer ring 311, which can break away and remain engaged with and implanted in the SI joint 102. In embodiments, the cannula 250 may be removed while leaving the guide wire 114 in place, and subsequent instruments and implants 400 may be threaded over the guide wire 114 to be directed to the implant site. The guidance tabs 315a, 315b may be operable to be removed from the washer ring 311 at connection points 314a, 314b. For example, the material of the guidance tabs 315a, 315b may be sufficiently thin and/or pre-treated (e.g., scored, thermally treated, chemically treated, or otherwise prepared) such that the guidance tabs 315a, 315b may be snapped or broken off at the connection points 314a, 314b such that they can be removed from the washer ring 311 and the incision over the SI joint 102. In other examples, the guidance tabs 315a, 315b can be removed by other techniques, e.g., by a shearing or cutting tool.

FIGS. 36k and 36l illustrate an example of how the guidance tabs 315a, 315b may be removed. The material joining the proximal ends of the guidance tabs 315a, 315b may be cut or otherwise sheared using a cutting tool. Subsequently, the guidance tabs 315a, 315b may be rotated outward from the washer ring 311 along a plane perpendicular to the washer ring 311, such that shearing force is applied at the connection points 314a, 314b causing the guidance tabs 315a, 315b to break away from the washer ring 311 at the connection points 314a, 314b. The guidance tabs 315a, 315b may then be removed from the incision, and the tissues in the incisions may then be sutured, to facilitate healing.

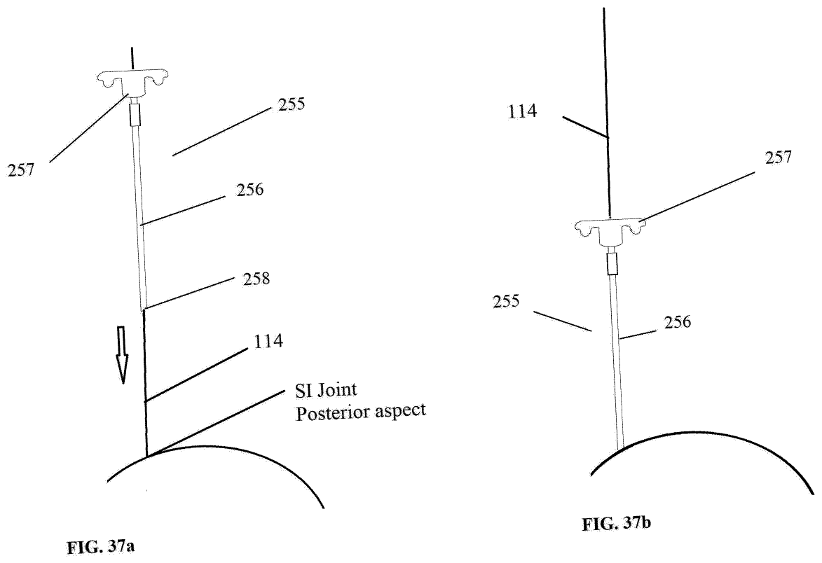

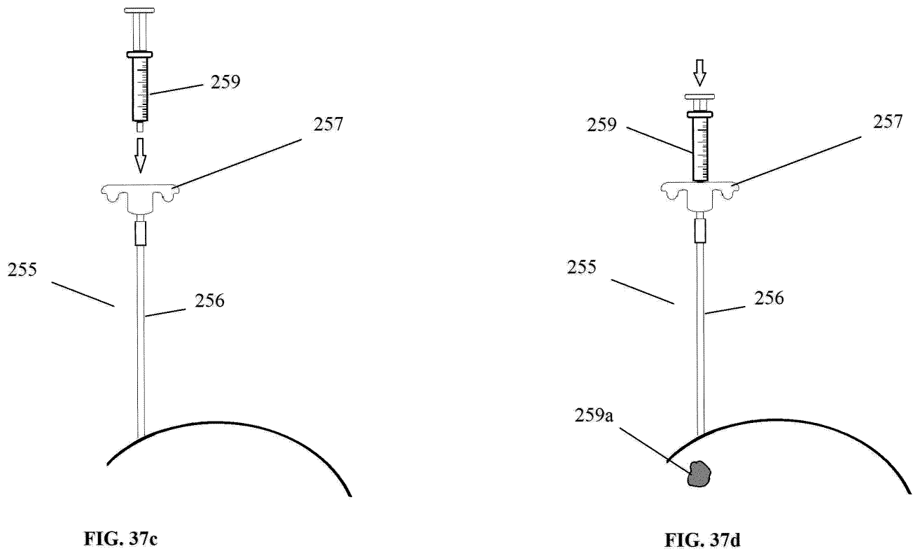

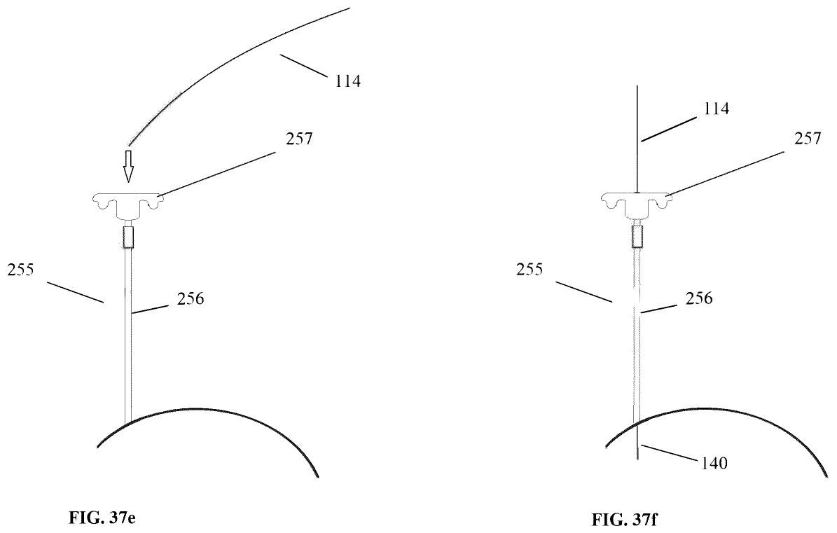

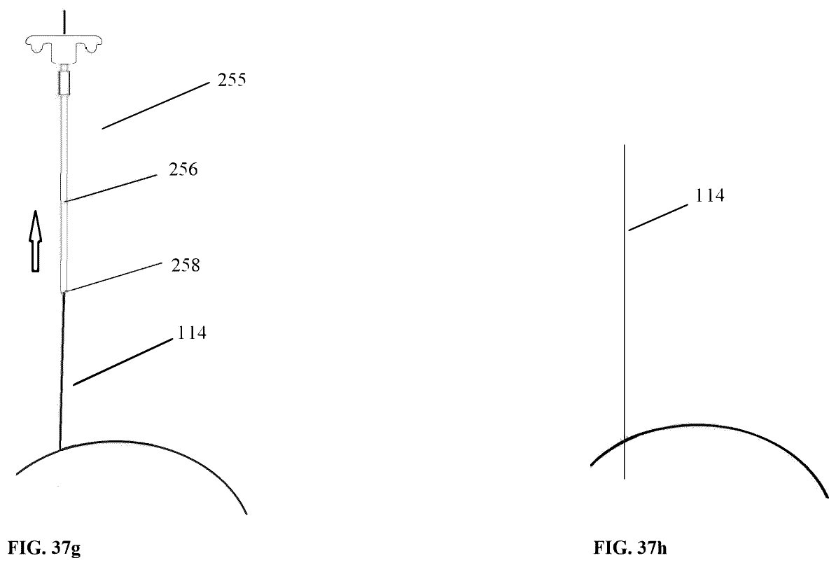

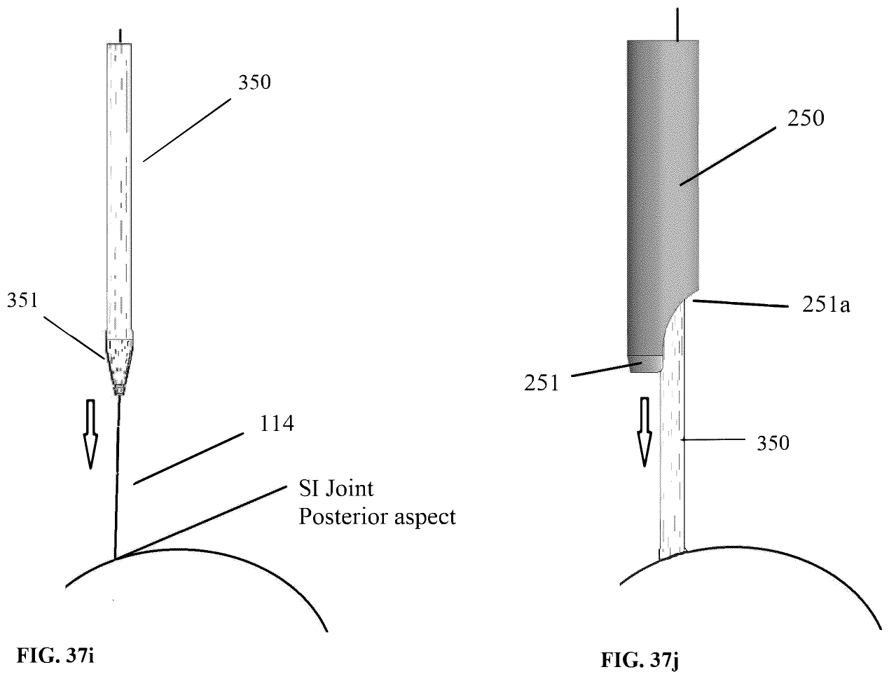

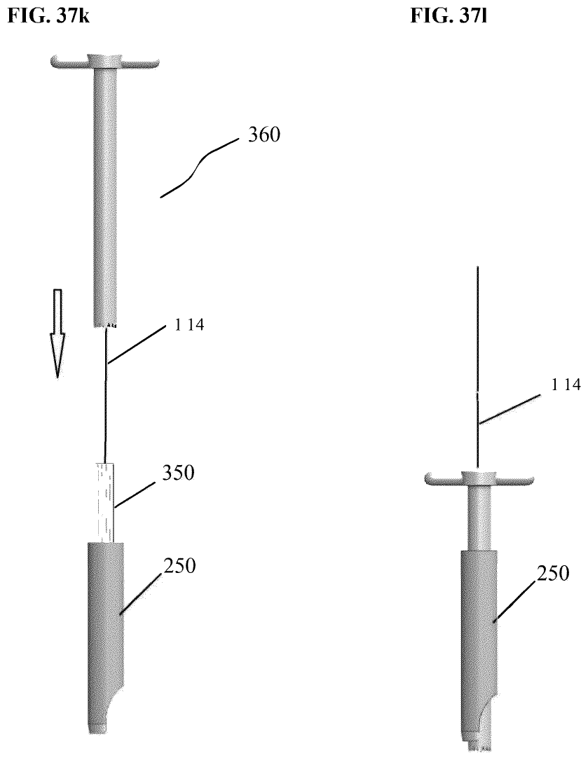

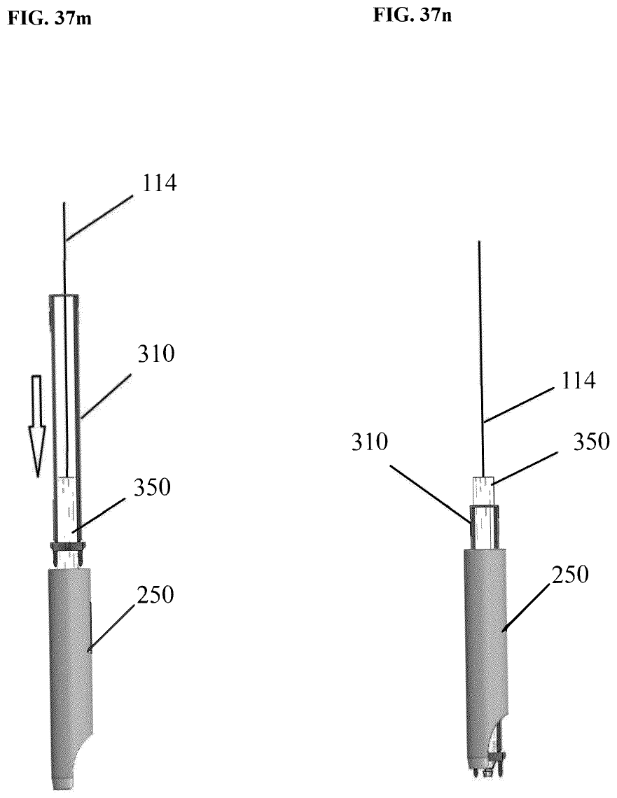

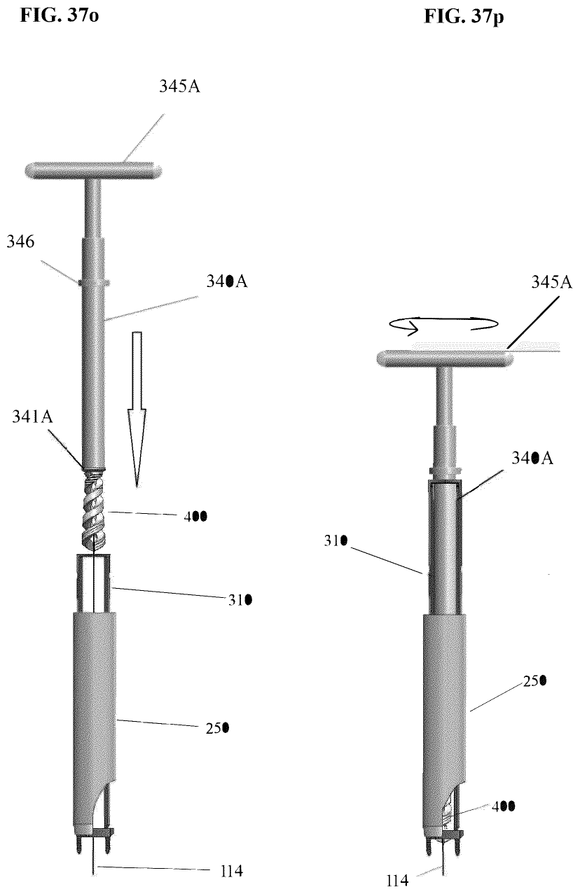

FIGS. 37a-37n illustrate another example surgical procedure for fusing an SI joint 102 with one or more fusion implants 400 inserted in the SI joint 102. The procedure includes positioning a patient in the prone position and administering either a local or general anesthetic. Blunt finger palpation may be used to locate the patient's iliac wing 100 and the SI joint 102. FIGS. 8-24 show tools that may be used to establish an incision and assist in establishing a void in the sacroiliac joint 102. A joint probe 112 may be used to identify the insertion area on the posterior side of the SI joint 102. The area of the SI joint 102 may be probed until the rounded geometry of the joint probe 112 finds or drops into the proper position in the SI joint 102, where an incision may be properly made. Subsequently, a guide wire 114 or guide pin 114 may be inserted through an aperture 113 in the joint probe 112 and into the patient to create an incision in the SI joint 102. Alternatively, the incision may be made by any suitable method, including scalpel or other cutting or dissection tool. The incision may be made proximate to the patient's SI joint 102, allowing the joint to be accessed by the exposure device. The guide wire 114 or guide pin 114 may be advanced until its distal end 115 is in contact with the SI joint 102 or at least partially within SI joint 102. The incision may be positioned between a posterior inferior iliac spine and a superior lateral aspect of the sacrum 101 of the SI joint 102. In other implementations, the incision may be positioned below a posterior superior iliac spine between the ilium 100 and sacrum 101 of the sacroiliac joint 102.

As shown in FIG. 37a-37b, a first cannula 255 may be threaded over the guide wire 114 or guide pin 114 and inserted within the incision. The first cannula 255 may be utilized to confirm the position of the guide pin 114 to confirm the placement of the guide pin 114 and the first cannula 255 in the joint space. The first cannula 255 may include a channel 256 running from the proximal handle 257 to the distal opening 258 of the first cannula 255. In some examples, the diameter of the channel 256 may be slightly larger than a guide pin 114 to allow the first cannula 255 to have relatively small size, which allows for insertion into the joint space over the guide pin 114.

In some embodiments, a radio-opaque dye 259a may be introduced through the first cannula 255 to confirm placement of the first cannula 255 in the joint space. As shown in FIGS. 37c-37d to introduce the radio-opaque dye 259a, the guide pin 114 may be removed from the joint to clear the channel 256 running through the first cannula 255, leaving the distal opening 258 of the first cannula 255 present in the joint space. The handle 257 may have a syringe receiver 255a that is in communication with the channel 256 to which a syringe 259 can be connected. A syringe 259 may contain a radio-opaque dye 259a, and a hub of the syringe 259 may be connected to the syringe receiver 255a, e.g., by pressure-fitting, threading, Luer lock, or other mechanism. Once the hub of the syringe 259 is connected to the syringe receiver 255a, the syringe 259 plunger may be depressed to inject the radio-opaque dye 259a through the channel 256 and into the joint space. Subsequently, CT scan, x-ray, or other imaging technique may be used to visualize the placement of the radio-opaque dye 259a. If it is present between the articular surfaces of the sacrum 101 and ilium 100, placement of the first cannula 255 is confirmed, and the surgical procedure can proceed. If the radio-opaque dye 259a is not found to be present in the joint space, the steps of locating the joint space may be repeated.

As shown in FIGS. 37e-37h, upon confirmation of the placement of the first cannula 255 in the joint space, the syringe 259 may be removed from the first cannula 255. A guide wire 114 may then be threaded through the first cannula 255 and into the joint space. Once the guide wire 114 is in place in the joint space, the first cannula 255 can be removed from the joint and passed over the guide wire 114, leaving the guide wire 114 present in the joint space.

As shown in FIG. 37i, a joint finder 350 may be slotted over guide wire 114 through a guide hole 353 running the length of the joint finder 350. The proximal end of the joint finder 350 may be slotted over the guide wire 114, and joint finder 350 may then be advanced to or near the SI joint 102 through the incision. As joint finder 350 enters the incision, the head 351 has a tapered shape with abrading surfaces 352a, 352b pushes the patient's tissue aside, thereby dilating incision to accommodate an exposure device as described herein. The guide wire 114 may then be removed from the joint space and passed through the channel 256 leaving the joint finder 350 in the joint space. A driver 370 may be coupled with the joint finder 350 and the driver 370 may be struck with a mallet or other appropriate instrument to drive the head 351 of the joint finder 350 into the SI joint 102. The abrading surfaces 352a, 352b may prepare the SI joint 102 for insertion of the joint fusion implant 400, clearing soft tissues from the joint and decorticating the articular surfaces.

FIG. 37j illustrates the placement of the cannula 250 over the guide wire 114. The cannula 250 can be positioned over the joint finder 350 and the guide wire 114 in the incision. The cannula 250 is advanced toward SI joint 102 through incision until distal end 251 is in contact with the SI joint 102 or proximal to the SI joint 102 and in contact with the sacrum 101 and/or ilium 100. The cannula 250 has distal end 251 with a round geometry and/or a tapered rounded profile that is operable to distract the SI joint 102 with minimal damage to soft and connective tissue in and around the posterior side of the SI joint 102. The cannula 250 may be stabilized using exterior features (not shown) through which fixing pins 126 may be slotted to stabilize the cannula 250. The cannula 250 may be stabilized by attachment to a surgical or stabilizing arm to hold the cannula 250 in a static and stable position.

FIGS. 37k-371 illustrate placing of the cylindrical drill 360 over the joint finder 350 into the incision through the cannula 250. The blades 363 of cylindrical drill 360 may be placed in contact with the bone tissue in the SI joint 102. Once the cylindrical blade 361 is placed in the SI joint 102, the cylindrical drill 360 may be rotated manually using handle 365. The cylindrical drill 360 may have cylindrical channel extending through the drill shaft 362 that allows the cylindrical drill 360 to be positioned over the joint finder 350 and freely spin around the joint finder 350. The drill shaft 362 may comprise a low-friction material that facilitates smooth spinning of the cylindrical drill 360 within the cannula 250. When advanced into the SI joint 102, the cylindrical drill 360 may decorticate the patient's sacrum 101 and ilium 100 in the SI joint 102. In such examples, the cylindrical drill 360 may be configured such that it will contact the patient's SI joint 102 at a desired portion of the joint and will remove a portion of the bone tissue to a desired depth to facilitate advancement of the fusion implant 400 into the bone. As an example, and without limiting the present disclosure, the fusion implant 400 may be one of those shown in FIGS. 25a-27f.

The present method may utilize additional implements that may be inserted through the cannula 250 and into the void in the SI joint 102 to prepare the joint for receiving a fusion implant 400. For instance, a chisel 131 and/or a rasp 132 may be inserted into the void through the cannula 250 to expand and clear tissue from the void to facilitate a clean and efficient insertion of the fusion implant 400 into the void (see, e.g., FIGS. 8-24).

As shown in FIGS. 37m-37n, a washer 310 may be passed over the joint finder 350 and inserted into the SI joint 102. The washer 310 includes a washer ring 311 at its distal end that may include lateral tangs 312a, 312b for penetrating tissues in the SI joint 102 and establishing a stable purchase of the washer 310 before a joint fusion implant 400 is inserted into the SI joint 102. The washer 310 may further include guidance tabs 315a, 315b that connect to the washer ring 311 at break-away connection points 314a, 314b. The diameter of the washer ring 311 and the distance between the guidance tabs 315a, 315b may be slightly larger than the diameter of the joint finder 350 to allow the washer 310 to be passed over the joint finder 350 into the SI joint 102. The driver 370 may be placed over the proximal end of the washer 310, which may be positioned within the canal 373. A mallet or other appropriate device may then be used to apply force to the head 371 to drive the tangs 312a, 312b into the articular space of the SI joint 102. The joint finder 350 is removed from the SI joint 102 once the washer 310 is in place in the SI joint 102 with the lateral tangs 312a, 312b engaged with the articular surfaces.

FIGS. 370-37p illustrate the use of an inserter 340A to insert a fusion implant 400 into the void in the SI joint 102. The inserter 340A is operable to engage with a joint fusion implant 400 to insert the joint fusion implant 400 into the targeted joint. The inserter 340A may be engaged with the head 402 of a joint fusion implant 400 and then used to advance the joint fusion implant 400 through the cannula 250 and into the SI joint 102. The inserter 340A may have a distal end 341A configured to attach to the joint fusion implant 400, e.g., having a bit-receiving recess 344 (e.g., star, hex, or other functional shape) for receiving a complementary head 402 of a joint fusion implant 400. The inserter 340A may have a mechanism for tightening the engagement of the bit recess 344 with the head 402 of the joint fusion implant 400. For example, the inserter 340A may have an actuator operable by the medical personnel to control a spring-operated element in the bit recess 344 that reversibly applies pressure to the head 402 of the joint fusion implant 400 to maintain the joint head 402 in the bit recess 344.

Once the inserter 340A has been advanced to the predetermined position at which the distal cutting edge 405 of the joint fusion implant 400 is at the superficial aspect of the SI joint 102, linear advancement of the inserter 340A may be paused, and the fusion implant 400 is positioned to be rotated while it is advanced, such that it engages and penetrates the articular surfaces of the bone to fix the articular surfaces relative to one another. The medical personnel may then manually rotate the inserter 340A using the handle 345A to cause the fixation element(s) of the joint fusion implant 400 to penetrate the bone tissue of the targeted joint.

The inserter 340A may have a radial ledge 346 on its proximal shaft that allows the medical personnel to monitor the depth to which the inserter 340A penetrates the SI joint 102. The radial ledge 346 may be used in conjunction with the guidance tabs 315a, 315b to determine when the inserter 340A has reached the desired depth to deliver a joint fusion implant 400 into the targeted joint. For example, the radial ledge 346 of the inserter 340 may be advanced until it meets the guidance tabs 315a, 315b to completely insert the fusion implant 400 into the targeted joint.

When the fusion implant 400 is fully advanced, the distal portion of the body 401 of the implant 400 may be nested in the complementary surface of the ring 311 of the washer 310. This provides a stable engagement between the washer 310 and the fusion implant 400. Once the fusion implant 400 has been fully inserted into the SI joint 102, the inserter 340A may be disengaged from the fusion implant 400 and removed from the cannula 250, and the cannula 250 and guide wire 114 can be removed from the SI joint 102, leaving the washer 310 exposed in the incision. The guidance tabs 315a, 315b can then be removed from the washer ring 311, as shown in FIGS. 36k-361. Subsequently, the tissues and incision may be sutured and bandaged.

### Interlock Method

In other embodiments, the method may involve the posterior insertion of a fusion implant 400, including the steps of creating an incision proximal to the patient's SI joint 102 102 (e.g., below a posterior superior iliac spine between the ilium 100 and sacrum 101 of the SI joint 102), introducing a guide pin 114, dilating the incision with a dilator 116, inserting a hybrid washer tower 500 into the incision over the dilator 116 or joint finder 350 and guide pin 114, creating a void in the SI joint 102, using an inserter 340 to pass a fusion implant 400 through the hollow barrel 510 of the hybrid washer tower 500 into the void such that the fusion implant 400 contacts the articular surfaces of the sacrum 101 and ilium 100, rotating the inserter 340 within the stationary hybrid washer tower 500 such that fusion implant 400 affixes the articular surfaces of the sacrum 101 and ilium 100 while the inserter 340 simultaneously couples with the hybrid washer tower 500, and removing the interlocked inserter 340 and hybrid washer tower 500 as a single unit. As discussed herein, a sensing device/technique can be used to locate the proper/suitable joint detection/placement in lieu of an initial guide pin 114 (e.g. a k-wire) for detection. Similarly, radio-opaque dye can be injected to effect joint confirmation, e.g., secondary joint confirmation. In this manner, once the joint location/placement is confirmed via the sensory mechanisms, a standard k-wire can be employed to commence the procedure as described herein.

In some embodiments, the method may include some or all of the following steps, preparing the patient for surgery (e.g., positioning the patient in a prone position to provide the surgeon access to the SI joint 102, providing general or local anesthesia, and the like), locating the SI joint 102 and an incision point for access to the SI joint 102, inserting a guide pin 114 or wire to create an incision, inserting a dilator 116 over the guide pin 114, impacting the dilator 116 to dilate the incision to a width through which instruments may be passed, inserting a hybrid washer tower 500 over the dilator 116, securing the hybrid washer tower 500 in position (e.g., by applying a force to a proximal end 502 of the hybrid washer tower 500 using a washer driver 330 to ensure the lateral tangs 512 positioned at a distal end 504 of the hybrid washer tower 500), removing the dilator 116, inserting a drill 260 through the hollow barrel 510 of the hybrid washer tower 500, rotating the drill 260 in the hollow barrel 510 to displace bone in the SI joint 102 thereby creating a void, removing the drill 260, loading a fusion implant 400 onto an inserter 340 and inserting the fusion implant 400 and inserter 340 through the hybrid washer tower 500 until the implant 400 is positioned proximal to the void in the patient's SI joint 102, rotating the inserter 340 and fusion implant 400 to engage the articular surfaces of the SI joint 102, as the inserter 340 rotates it simultaneously interlocks the hybrid washer tower 500, removing the interlocked inserter 340 and hybrid washer tower 500, and closing the incision.

In some embodiments, the method may include some or all of the following steps, preparing the patient for surgery (e.g., positioning the patient in a prone position to provide the surgeon access to the SI joint 102, providing general or local anesthesia, and the like), locating and marking the SI joint 102, inserting a guide pin 114 or wire to create an incision, inserting a first hybrid washer tower 500 over the guide pin 114 or wire into the joint space, injecting a radio-opaque dye 259a through the first hybrid washer tower 500 into the joint space, conducting an imaging process to visualize the dye in the joint to confirm placement of the first hybrid washer tower 500 in the joint space, removing the first hybrid washer tower 500, inserting a dilator 116 over the guide pin 114, impacting the dilator 116 to dilate the incision to a width through which instruments may be passed, inserting a hybrid washer tower 500 over the dilator 116, securing the hybrid washer tower 500 in position (e.g., by applying a force to a proximal end 502 of the hybrid washer tower 500 using a washer driver 330 to ensure the lateral tangs 512 positioned at a distal end 504 of the hybrid washer tower 500 engage the tissue surrounding the implant site), removing the dilator 116 and the guide pin 114, inserting a drill 260 through the hollow barrel 510 of the hybrid washer tower 500, rotating the drill 260 in the hollow barrel 510 to displace bone in the SI joint 102 thereby creating a void, removing the drill 260, loading a fusion implant 400 onto an inserter 340 and inserting the fusion implant 400 and inserter 340 through the hybrid washer tower 500 until the fusion implant 400 is positioned proximal to the void in the patient's SI joint 102, rotating the inserter 340 and fusion implant 400 to engage the articular surfaces of the SI joint 102, as the inserter 340 rotates it simultaneously interlocks with the hybrid washer tower 500, removing the interlocked inserter 340 and hybrid washer tower 500, and closing the incision. The radio-opaque dye 259a may include various materials such as iodine, indigo carmine, lipiodol, indocyanine green, and mixtures thereof. The radio-opaque dye 259a position may be imaged using computerized tomography scan (CT), X-ray imaging, or other appropriate imaging methods.

The methods of the present disclosure according to the embodiments discussed herein are now further described with reference to the Figures. Although the methods are described with respect to the use of certain tools, the methods of the present disclosure may be performed in addition to or in conjunction with one or more of the known other tools used in the relevant art.

FIGS. 41-42 and FIGS. 43a-43j illustrate an example surgical procedure for fusing an SI joint 102 with one or more fusion implants 400 inserted in the SI joint 102. The procedure includes positioning a patient in the prone position and administering either a local or general anesthetic. Blunt finger palpation may be used to locate the patient's iliac wing 100 and the SI joint 102, as discussed herein. Alternatively, suitable locations for an incision may be determined by imaging methods (e.g., X-ray), or any other suitable method. The incision may be positioned between a posterior inferior iliac spine and a superior lateral aspect of the sacrum 101 of the SI joint 102. In other implementations, the incision may be positioned below a posterior superior iliac spine between the ilium 100 and sacrum 101 of the sacroiliac joint 102.

FIGS. 8-24 and FIGS. 41-42 show tools that may be used to establish an incision and assist in establishing a void in the sacroiliac joint 102. A joint probe 112 may be used to identify the insertion area on the posterior side of the SI joint 102. The area of the SI joint 102 may be probed until the rounded geometry of the joint probe 112 finds or drops into the proper position in the SI joint 102, where an incision may be properly made. Subsequently, a guide pin 114 may be inserted through an aperture 113 in the joint probe 112 and into the patient to create an incision in the SI joint 102. Alternatively, the incision may be made by any suitable method, including scalpel or other cutting or dissection tool. The incision may be made proximal to the patient's SI joint 102, allowing the joint to be accessed by the exposure device. The guide pin 114 may be advanced until its proximal end 502 is in contact with the SI joint 102 or at least partially within SI joint 102.

As shown in FIGS. 43a and 43b, a dilator 116 may be used to dilate the incision. As an example, the dilator 116 may be slotted over the guide pin 114 through a column 213 running the length of the dilator 116. The proximal end 502 of the dilator 116 may be slotted over the guide pin 114, and dilator 116 may then be advanced to or near the SI joint 102 through the incision. As dilator 116 enters the incision, the tapered distal end 118 pushes the patient's flesh and tissue aside, thereby dilating the incision to accommodate an exposure device as described herein. A joint cutting assembly that includes the dilator 116 and a T-handle 119 (FIG. 16) engaged with a head 351 of the dilator 116 may be used to further drive the dilator 116 into the incision to a desired depth to sufficiently expose the SI joint 102. Alternatively, an impactor 136 may be used to further drive the dilator 116 into the incision to a desired depth.

FIG. 41 and FIG. 43c-43j illustrate the hybrid washer tower 500. The hybrid washer tower 500 may have a closed or semi-closed columnar outer wall 515 that defines a hollow barrel 510 extending from a proximal end 502 to a distal end 504. In embodiments, the hybrid washer tower 500 may include lateral tangs 512 for penetrating tissues surrounding the implant site before an instrument or a joint fusion implant 400 is passed through the hollow barrel 510 of the hybrid washer tower 500 toward the SI joint 102. The hybrid washer tower 500 may further include interlock channels 516 positioned at the proximal end 502. The interlock channels 516 may serve multiple purposes during the insertion of a fusion implant 400. In embodiments, the interlock channels 516 may be operable to be engaged with a washer driver 330 in order to operatively couple the hybrid washer tower 500 and the washer driver 330. In embodiments, the interlock channels 516 may serve as reference points to allow the user to control the orientation of an instrument or fusion implant 400 and to gauge the depth of an instrument within the incision based on the instrument's relative position compared to the interlock channels 516. In further embodiments, the interlock channels 516 may engage with one or more connection pins 518 positioned on the shaft 348 of the inserter 340.

FIG. 43a-43c illustrates the placement of the hybrid washer tower 500 over the dilator 116 and the guide pin 114. Subsequently, the dilator 116 and the guide pin 114 may be removed from the incision through the hollow barrel 510 of the hybrid washer tower 500. In embodiments, the guide pin 114 may be left in place throughout the procedure, and subsequent instruments and implants 400 may be threaded over the guide pin 114 to be directed to the implant site. The hybrid washer tower 500 may removably couple to a washer driver 330 that may surround the proximal end 502 of the hybrid washer tower 500 to allow a user to advance the hybrid washer tower 500 further into the incision. The hybrid washer tower 500 is advanced toward SI joint 102 through incision until distal end 504 is in contact with the SI joint 102 or is positioned proximal to the SI joint 102 and in contact with the sacrum 101 and/or ilium 100. In embodiments, the distal end 504 has a round geometry and/or a tapered rounded profile that may engage the SI joint 102 with minimal damage to soft and connective tissue in and around the posterior side of the SI joint 102. In other embodiments, the distal end 504 of the hybrid washer tower 500 has a circular cross-section with no recessed portions. It should be understood that the hybrid washer tower 500 may have other cross-sectional shapes as well (e.g., elliptical, polygonal [pentagonal, hexagonal, etc.], Reuleaux shapes, and other applicable shapes to accommodate the shape of various fusion implants 400). Additionally, the hybrid washer tower 500 may include lateral tangs 512 or other protrusions extending from a terminal portion of the distal end 504 to engage the tissue surrounding the implant site and resist movement. The hybrid washer tower 500 may also be used to further dilate the incision.

FIG. 43f illustrates insertion of drill 260 into the incision through the hybrid washer tower 500. The drill 260 may be connected to a power tool configured for medical procedures. The drill 260 may have cylindrical outer walls that allow the drill 260 to freely spin within the hollow barrel 510 of the hybrid washer tower 500. The cylindrical outer wall 515 may comprise a low-friction material that facilitates smooth spinning of the drill 260 within the hollow barrel 510. The proximal end 502 of the drill 260 may be inserted into the hybrid washer tower 500 over guide wire 114 and may be advanced to a predetermined point. In some embodiments, the stepped blade portion 263 of the drill 260 extend past the distal end 504 of the hybrid washer tower 500 by a pre-determined amount that is equal to or less than a length of an insertable portion of a fusion implant 400 (e.g., fusion implant 400, 450, 470, etc.). The drill 260 may have a shoulder 262 that provides indication of the depth to which the drill 260 should be advanced into the SI joint 102. When the shoulder 262 meets the proximal end 502 of the hybrid washer tower 500, the drill 260 may be advanced to the desired pre-determined depth. When advanced into the SI joint 102, the drill 260 may create a void in the patient's SI joint 102 by displacing portions of sacrum 101 and ilium 100. In such examples, the drill 260 may be configured such that it will contact the patient's SI joint 102 at a desired portion of the joint and, once activated, will create a void of a desired depth. The void may be configured to receive a fusion implant 400, other joint repairing appliance, or bone graft as described herein for fusing the SI joint 102. Other joint repairing appliances or apparatus may include a polyether ether ketone (PEEK) implant, a titanium implant, etc.

The subject method may utilize several implements that may be inserted through the hybrid washer tower 500 and into the SI joint 102 to prepare the void for receiving a fusion implant 400. For instance, a box chisel 131 (FIG. 22) and/or a rasp 132 (FIG. 23) may be inserted into the void through the hybrid washer tower 500 to expand and clear tissue for efficient insertion of the fusion implant 400 at the implant site.

FIG. 42 and FIGS. 43g-43i illustrate the inserter 340 and the use of the inserter 340 to transport a fusion implant 400 through the hollow barrel 510 and into the implant site of the SI joint 102. The inserter 340 may be inserted through the hollow barrel 510 of the hybrid washer tower 500 once the washer driver 330 has been decoupled from the hybrid washer tower 500. The inserter 340 is operable to engage with a joint fusion implant 400 so that the joint fusion implant 400 may be affixed to a distal end 341 of the inserter 340 to allow a user to implant the fusion implant 400 into the targeted joint. The distal end 341 of inserter 340 may engage with the head 402 of a joint fusion implant 400 to advance the joint fusion implant 400 through the hybrid washer tower 500 and into the targeted joint. The inserter 340 may have a distal end 341 configured to attach to the joint fusion implant 400, e.g., having a bit-receiving recess 344 (e.g., star, hex, or other functional shape) for receiving a corresponding head 402 of a joint fusion implant 400. The inserter 340 may have a mechanism for tightening the engagement of the bit recess 344 with the head 402 of the joint fusion implant 400. For example, an actuator operable by the medical personnel may control a spring-operated element, e.g., in the bit recess 344 that reversibly applies pressure to the head 402 of the joint fusion implant 400 to maintain the head 402 in the bit recess 344.

As shown in FIG. 42 and FIGS. 43g-43j, the inserter 340 may be advanced along a longitudinal axis (i.e. linear advancement) within the hybrid washer tower 500 to a predetermined, terminal position. The terminal position is reached when one or more connection pins 518 positioned on the shaft 348 of the inserter 340 arrest the longitudinal movement (i.e. linear advancement) of the inserter 340 through the hollow barrel 510 of the hybrid washer tower 500. The movement and orientation of the inserter 340 may be restricted within the hybrid washer tower 500 by one or more interlock channels 516. The interlock channel 516 may be an L-shaped recess defined on the interior of the hollow barrel 510 or an L-shaped open channel 516 disposed within the outer wall 515 and positioned at a terminal portion of the proximal end 502 of the hybrid washer tower 500. The interlock channel 516 is configured accept the connection pin 518 on the inserter shaft 348 such that the longitudinal movement of the inserter 340 may be arrested when the connection pin 518 progresses though and reaches the end of a longitudinal portion 520 of the interlock channel 516. Medical personnel may use the terminal, longitudinal position as a reference to ensure that the fusion implant 400 is positioned at the superficial aspect of the SI joint 102. Once the inserter 340 has progressed through the hollow barrel 510 of the hybrid washer tower 500 such that the connection pin 518 on the inserter shaft 348 has reached its terminal, longitudinal position, medical personnel may then rotate the inserter 340 (for example, ninety degrees) within the hybrid washer tower 500. Rotation may be either clockwise or counter-clockwise to correspond with the configuration of the fusion implant 400 and the interlock channels 516. The rotation of the inserter 340 allows medical personnel to finalize the fusion implant 400 position within the SI joint 102, such that it causes the fixation element(s) of the joint fusion implant 400 to penetrate and affix the bone tissue of the targeted joint. As the fusion implant 400 is being positioned within the implant 400 site due to the ninety degree rotation of the inserter 340, the connection pin 518 is moved from the longitudinal portion 520 of the interlock channel 516 to the transverse portion 522 of the interlock channel 516. Accordingly, a single ninety degree turn of the inserter 340 within the hybrid washer tower 500 may finalize a position of the fusion implant 400 within the SI joint 102, while simultaneously interlocking the inserter 340 with the hybrid washer tower 500 so that the inserter 340 and hybrid washer tower 500 may now move in unison when a longitudinal force is applied.

Once the fusion implant 400 has been fully inserted into the SI joint 102, the inserter 340 may be disengaged from the fusion implant 400. Medical personnel may then apply a longitudinal force (i.e. a linear pulling force) to the inserter 340 to remove it from the incision. Due to the interlocked connection pin 518 and interlock channel 516, the application of a longitudinal force to the inserter 340 may remove both the inserter 340 and the hybrid washer tower 500 in a single motion. In so doing, only the fusion implant 400 is left within incision and all remaining instruments and components of the method are simultaneously removed. Subsequently, the tissues and incision may be sutured and bandaged.

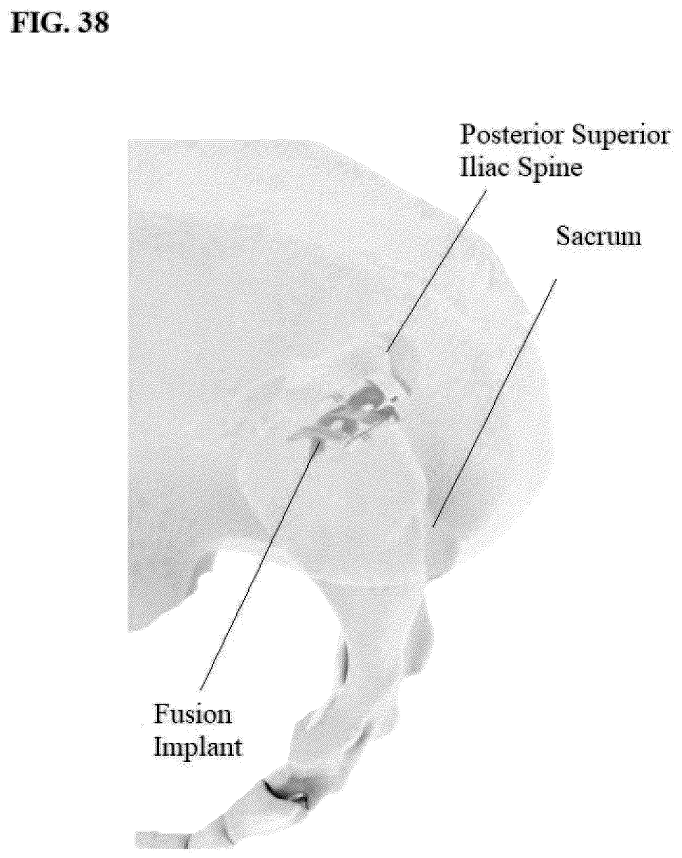

FIG. 38 provides a view of a fusion implant 400 in position in the SI joint 102 after surgical implantation. The pelvis 201 is presented as semi-transparent for illustration purposes. The fusion implant 400 is positioned about between a superior lateral aspect of the sacral wing and the posterior superior iliac spine of the ilium 100. This is an appropriate and effective location for implantation between the articular surfaces of the SI joint 102, which can accomplish an effective fusion of the SI joint 102 in a single implant 400 approach. However, the present disclosure is not limited to such placement in the SI joint 102. For example, the fusion implant 400 may be positioned at or near an inferior aspect of the sacroiliac joint 102.

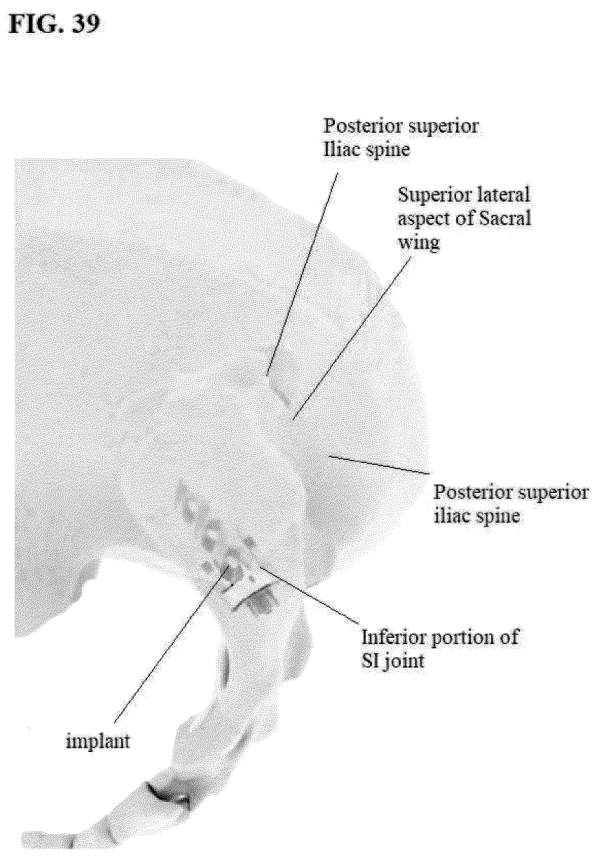

FIG. 39 provides a view of a fusion implant 400 in position in the SI joint 102 after surgical implantation in another implementation. The fusion implant 400 is positioned below the posterior superior iliac spine at or near an inferior aspect of the sacroiliac joint 102. This is an appropriate and effective location for implantation between the articular surfaces of the SI joint 102, which can accomplish an effective fusion of the SI joint 102 in a single implant 400 approach. However, the present disclosure is not limited to such placement in the SI joint 102.

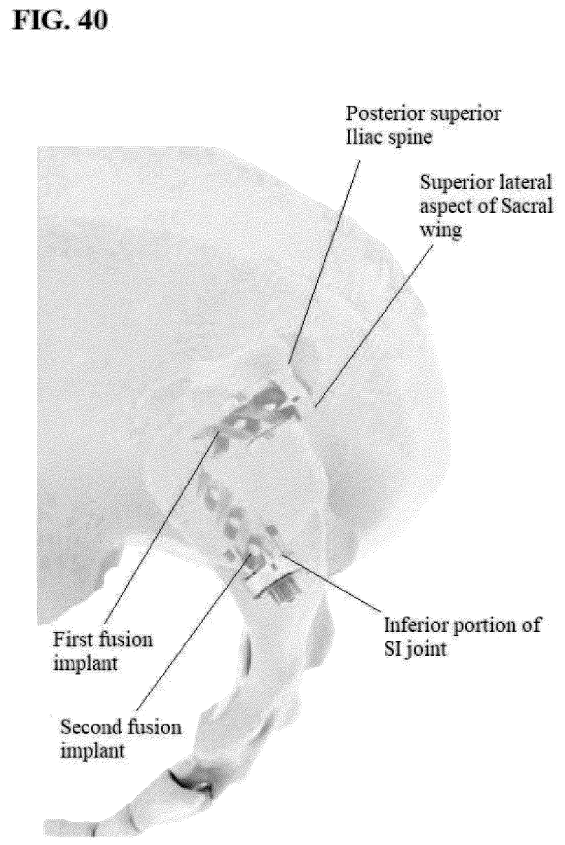

Additionally, the present disclosure is not limited to methods that insert a single fusion implant 400 into an SI joint 102. FIG. 40 shows the placement of two fusion implants 400 in an SI joint 102, a first implant 400 positioned about between a superior lateral aspect of the sacral wing and the posterior superior iliac spine of the ilium 100, and a second implant 400 positioned below the posterior superior iliac spine at or near an inferior aspect of the sacroiliac joint 102. The second implant 400 may be implanted in the SI joint 102 by the methods described herein. The methods may be carried out at two different locations in the joint.

It is also to be appreciated that the tools, devices, and methods disclosed herein are not limited to SI joint 102 fusion procedures, and may have other beneficial applications. It is to be further appreciated that the tools, devices, and methods disclosed herein can be utilized in SI joint 102 fusion procedures on both SI joints of a patient simultaneously.

It is intended to be understood that embodiments include positioning instruments underneath the Posterior Superior Illiac Spine (PSIS) as well as use of a sensing device(s) and/or technique(s) to locate a suitable or appropriate joint space. It is further to be understood that by placing the washer 310 beneath the PSIS, it abuts against the PSIS and allows for resistance of nutation. As further described herein, the screw resists other motions as well, thereby providing effective fixation (e.g., motion lockdown). Additionally, in some aspects, radio-opaque dye can be injected, for example via a syringe 259 into a depth/location sensor-equipped cannula 250 or other suitable methods/devices, so as to facilitate joint placement confirmation (e.g., secondary confirmation). These alternatives are to be included within the spirit and scope of this present disclosure and claims appended hereto.

It may be noted that one or more of the following claims utilize the terms “where,” “wherein,” or “in which” as transitional phrases. For the purposes of defining the present technology, it may be noted that these terms are introduced in the claims as an open-ended transitional phrase that are used to introduce a recitation of a series of characteristics of the structure and should be interpreted in like manner as the more commonly used open-ended preamble term “comprising.”

Having described the subject matter of the present disclosure in detail and by reference to specific embodiments, it may be noted that the various details described in this disclosure should not be taken to imply that these details relate to elements that are essential components of the various embodiments described in this disclosure, even in casings where a particular element may be illustrated in each of the drawings that accompany the present description. Rather, the claims appended hereto should be taken as the sole representation of the breadth of the present disclosure and the corresponding scope of the various embodiments described in this disclosure. Further, it will be apparent that modifications and variations are possible without departing from the scope of the appended claims.

1. A hybrid washer tower for posterior surgical stabilization of a sacroiliac joint, the hybrid washer tower comprising:
a columnar outer wall extending from a distal end to a proximal end, the columnar outer wall defining a hollow barrel extending from the distal end to the proximal end, the hollow barrel being at least partially enclosed by the outer wall;
at least one lateral tang positioned on and projecting from a terminal portion of the distal end of the outer wall to engage with surrounding bone or tissue; and
an interlock channel defined within the outer wall having an open section positioned at a terminal portion of the proximal end of the outer wall to accept a connection pin positioned on a surgical instrument and direct the movement of the surgical instrument as it progresses through the hollow barrel.

2. The hybrid washer tower of claim 1, further comprising two lateral tangs diametrically positioned on the terminal portion of the distal end, wherein each of the lateral tangs comprises a plurality of serrations, barbs, hooks, splines, or ridges positioned on a distal edge of each lateral tang.

3. The hybrid washer tower of claim 1, wherein each lateral tang further comprises one or more surface features on an exterior surface to facilitate a stable placement.

4. The hybrid washer tower of claim 1, wherein the hollow barrel has a circular cross-section configured to enable passage of one or more surgical instruments to an implant site.

5. The hybrid washer tower of claim 1, wherein the interlock channel has a corresponding width and depth to accept and guide the movement of a connection pin positioned on the shaft of a fusion implant inserter.

6. The hybrid washer tower of claim 1, wherein the interlock channel has a corresponding width and depth to accept and operatively couple with a washer driver to enable further positioning of the hybrid washer tower via a driving force.

7. The hybrid washer tower of claim 1, wherein the interlock channel comprises a longitudinal portion extending along the length of the outer wall and a transverse portion extending perpendicular to the longitudinal portion, wherein the longitudinal portion and the transverse portion integrally meet to form a single L-shaped channel.

8. The hybrid washer tower of claim 7, wherein the size and dimensions of the longitudinal portion and the transverse portion are predetermined to correspond to the depth and orientation of an implant site, wherein the longitudinal portion further comprises a terminal position that can arrest the longitudinal movement of the connection pin and prevent over insertion of the surgical instrument.

9. The hybrid washer tower of claim 7, wherein the surgical instrument moves independently through the hybrid washer tower when the connection pin is positioned within the longitudinal portion of the interlock channel, but the surgical instrument and hybrid washer tower are interlocked when the connection pin is positioned within the transverse portion of the interlock channel, enabling simultaneous removal of the surgical instrument and the hybrid washer tower from an implant site.

10. The hybrid washer tower of claim 7, wherein the transverse portion is configured to allow rotation of connection pin of the surgical instrument within the hollow barrel in a corresponding direction to a fixation element of a fusion implant.

11. A method for the posterior surgical stabilization of a sacroiliac joint using a hybrid washer tower, the method comprising the steps of:
creating an incision proximal to a patient's sacroiliac joint to allow access to a posterior region of the sacroiliac joint;
locating an implant site and introducing a guide pin to mark the implant site;
fitting a dilator over the guide pin and dilating the incision;
threading a hollow barrel of a hybrid washer tower over the dilator and progressing a distal end of the hybrid washer tower into the dilated incision;
removing the dilator and the guide pin through the hollow barrel of the hybrid washer tower, leaving the hybrid washer tower positioned within the dilated incision to enable the passage of one or more surgical instruments and one or more implants through the hollow barrel to the implant site;
inserting a drill through the hollow barrel until one or more blades of the drill are positioned proximate to the implant site, rotating the blades to create a void in the patient's sacroiliac joint, and removing the drill through the hollow barrel,
coupling the implant to a distal end of an inserter, inserting the distal end of the inserter into the hollow barrel, orienting the inserter so that a connection pin positioned on a shaft of the inserter enters an interlock channel defined within an outer wall of the hybrid washer tower, and progressing the inserter through the hollow barrel until the distal end of the inserter and the implant are positioned proximate to the void;
rotating the inserter within the hollow barrel such that the rotation of the inserter affixes the implant within the sacroiliac joint, while simultaneously interlocking the inserter with hybrid washer tower;
decoupling the implant from the inserter and applying a longitudinal force to a handle of the inserter to remove the interlocked inserter and hybrid washer tower from the incision simultaneously, without leaving any break away components; and
suturing the incision.

12. The method of claim 11, wherein the interlock channel comprises a longitudinal portion extending along the length of the outer wall and a transverse portion extending perpendicular to the longitudinal portion, wherein the longitudinal portion and the transverse portion integrally meet to form a single L-shaped channel.

13. The method of claim 12, wherein the size and dimensions of the longitudinal portion and the transverse portion are predetermined to correspond to the depth and orientation of an implant site, wherein the longitudinal portion further comprises a terminal position that can arrest the longitudinal movement of the connection pin and prevent the over insertion of the inserter.

14. The method of claim 12, wherein the inserter moves independently through the hybrid washer tower when the connection pin is positioned within the longitudinal portion of the interlock channel, but the surgical instrument and hybrid washer tower are interlocked when the connection pin is positioned within the longitudinal portion of the interlock channel, enabling simultaneous removal of the inserter and the hybrid washer tower from an implant site.

15. The method of claim 11, wherein a ninety-degree rotation of the inserter can simultaneously affix the implant to the sacroiliac joint while interlocking the inserter and the hybrid washer tower.

16. The method of claim 11, wherein the hybrid washer tower further comprises two lateral tangs diametrically positioned on a terminal portion of a distal end of the hybrid washer tower, wherein each of the lateral tangs comprises a plurality of serrations, barbs, hooks, splines, or ridges positioned on a distal edge of each lateral tang.

17. The method of claim 11, wherein locating the implant site further comprises inserting a first cannula over the guide pin, injecting a radio-opaque dye into the implant site through the first cannula, removing the first cannula, and imaging via a computerized tomography scan or an x-ray to confirm a position of the implant site.

18. The method of claim 11, further comprising the step of coupling a washer driver to a proximal end of the hybrid washer tower following the removal of the dilator and the guide pin, wherein the washer driver comprises a flat, proximal end configured to accept a driving force.

19. The method of claim 18, wherein the interlock channel has a corresponding width and depth to accept and operatively couple with the washer driver to enable further positioning of the hybrid washer tower within the dilated implant site via a driving force.

20. A hybrid washer tower for posterior surgical stabilization of a sacroiliac joint, the hybrid washer tower comprising:
a columnar outer wall extending from a distal end to a proximal end, the columnar outer wall defining a hollow barrel extending from the distal end to the proximal end, the hollow barrel being at least partially enclosed by the outer wall;
two lateral tangs positioned on and projecting from a terminal portion of the distal end of the outer wall to engage with a bone or tissue, wherein the two lateral tangs are diametrically positioned on the terminal portion of the distal end, wherein each of the lateral tangs comprises a plurality of serrations, barbs, hooks, splines, or ridges positioned on a distal edge of each lateral tang; and
an interlock channel defined within the outer wall having an open section positioned at a terminal portion of the proximal end of the outer wall to accept a connection pin positioned on a surgical instrument and direct the movement of the surgical instrument as it progresses through the hollow barrel, the interlock channel comprising a longitudinal portion extending along the length of the outer wall and a transverse portion extending perpendicular to the longitudinal portion,
wherein the longitudinal portion and the transverse portion integrally meet to form a single L-shaped channel such that the surgical instrument moves independently through the hybrid washer tower when the connection pin is positioned within the longitudinal portion of the interlock channel, but the surgical instrument and hybrid washer tower are interlocked when the connection pin is positioned within the transverse portion of the interlock channel, enabling simultaneous removal of the surgical instrument and the hybrid washer tower from an implant site.
